(19) United States
(12) Patent Application Publication
(10) Pub. No.: US 2016/0289719 A1
(43) Pub. Date: Oct. 6, 2016

(54) MODIFICATION OF THE XYLAN UTILIZATION SYSTEM FOR PRODUCTION OF ACIDIC XYLOOLIGOSACCHARIDES FROM LIGNOCELLULOSICS

(71) Applicant: UNIVERSITY OF FLORIDA RESEARCH FOUNDATION INCORPORATED, Gainesville, FL (US)

(72) Inventor: JAMES FAULKNER PRESTON, III, MICANOPY, FL (US)

(21) Appl. No.: 15/035,530

(22) PCT Filed: Nov. 25, 2014
(86) PCT No.: PCT/US14/67222

§ 371 Date: May 10, 2016

Related U.S. Application Data
(60) Provisional application No. 61/908,426, filed on Nov. 25, 2013.

(51) Int. Cl. C12P 19/14; C07H 3/06; C12N 9/24; C12P 19/04

(52) U.S. Cl. CPC C12P 19/14; C12P 19/04; C07H 3/06; C12N 9/2402

(57) ABSTRACT

The subject invention pertains to genetically modified microorganisms, e.g., genetically modified B. subtilis strain 168, that lack or which comprise an inactivated secreted endoxylanase of glycoside hydrolase family (GH) 10 or a homolog thereof, if present within the genome of the microorganism; that lack or comprise an inactivated secreted endoxylanase of GH11 or a homolog thereof, if present within the genome of the microorganism, and/or that lack or comprise an inactivated secreted endoxylanase belonging to GH 30 or a homolog thereof, if present within the genome of the microorganism. The current invention also pertains to a method of producing xylooligosaccharides with or without arabinofuranosyl substitutions (XOS and A-XOS), and/or acidic derivatives thereof (U-XOS and U-AXOS), the method comprising culturing the microorganisms of the current invention in a culture medium comprising methylglucuronoxylans (MeGXₙ) and/or methyl glucronoarabinoxylans (MeGAXₙ) under conditions that allow conversion of MeGXₙ and/or MeGAXₙ to XOS, AXOS, U-XOS, and/or U-AXOS.

### CROSS-REFERENCE TO RELATED APPLICATION

This application claims the benefit of U.S. Provisional Application Ser. No. 61/908,426, filed Nov. 25, 2013, the disclosure of which is hereby incorporated by reference in its entirety, including all figures, tables and amino acid or nucleic acid sequences.

This invention was made with government support under Grant No. 2011-10006-30358 awarded by The National Institute of Food and Agriculture. The government has certain rights in the invention.

### BACKGROUND OF THE INVENTION

Xylooligosaccharides without (XOS) and with (AXOS) arabinofuranosyl substitutions are of interest as value-added products derived from the hemicellulose fractions of lignocellulosics. There is evidence supporting the applications of these neutral forms comprised of β-1,4-linked xylose residues as prebiotics (1-3) and anti-inflammatory agents (4). Aldouronates, acidic xylooligosaccharides (U-XOS and U-AXOS) in which some xylose residues are substituted with α-1,2-linked 4-O-methylglucuronate (MeG), have been shown to exhibit anti-inflammatory and other immunomodulating activities (5). These acidic forms also comprise a portion of the pentosans that are used for the preparation of pentosan polysulfates which have several medical applications, including the treatment of interstitial cystitis, mucopolysaccharidoses, and osteoarthritis (6-8). The generation of different forms of XOS and AXOS or U-XOS and U-AXOS results from the depolymerization of both methylglucuronoxylans (MeGXₙ) and methylglucuronoarabinoxylans (MeGAXₙ), the predominant polymers comprising the hemicellulose fractions of lignocellulosics derived from hardwoods and grasses, respectively (9, 10).

The production of neutral and acidic forms can be achieved with endoxylanases of glycoside hydrolase families 10, 11 and 30 (see World Wide Website: cazy.org) as depicted in FIG. 1. Members of each family have been defined with respect to structure and function (11-16).

With MeGXₙ as substrate, GH10 xylanases generate xylobiose (X₂) and xylotriose (X₃) as XOS, and the aldotetrauronate 4-O-methylglucuronoxylotriose (MeGX₃) as U-XOS, in which a single MeG substitution occurs on the non-reducing terminal xylose (FIG. 1). The products of the GH10 enzymes may be assimilated and processed for the complete metabolism of the xylose and MeG components of the MeGXₙ. This intracellular processing depends upon the presence of a GH67 α-glucuronidase that cleaves the α-1,2-linked MeG from the non-reducing terminal xylose on the MeGX₃ generated by the GH10 xylanase. Exhaustive treatment of MeGXₙ with GH11 xylanase generates X₂ and X₃ as XOS and the aldopentauronate methylglucuronoxylotetraose (MeGX₄) with a single MeG substitution on the xylose penultimate to the non-reducing terminal xylose (11). This aldouronate is not a substrate for a GH67 α-glucuronidase, and MeGX₄ may accumulate as a limit product in media of bacterial cultures secreting only a GH11 endoxylanase. With MeGXₙ as substrate, GH30 xylanases generate exclusively aldouronates in which a MeG substitution occurs on a xylose residue penultimate to the reducing terminal xylose, producing U-XOS (14-17). These aldouronates may contain a variable number of xylose residues depending upon the distribution of MeG substitutions in the polymeric MeGXₙ (FIG. 1). As in the case of the MeGX₄ generated by GH11 endoxylanases, the position of the MeGA substitution does not allow processing by a GH67 α-glucuronidase.

Bacterial strains that contain these enzymes, for example, Bacillus subtilis strain 168 and other B. subtilis strains, can secrete a GH11 and a GH30 endoxylanase (16, 18). Such bacterial strains can be genetically modified to make biocatalysts useful in producing XOS, AXOS, U-XOS, and U-AXOS from MeGXₙ and/or MeAGXₙ.

Both GH11 and GH30 endoxylanases produced by B. subtilis strains have been well characterized with respect to products formed and structure/function relationships (15, 19, 20). For example, based upon analysis of the sequenced genome of B. subtilis strain 168, GH11 and GH30 are the only endoxylanases for which structural genes have been identified in this strain. With a fully sequenced genome, genetically malleable B. subtilis strain 168 can be genetically modified for the selective production of XOS, AXOS, U-XOS, and U-AXOS from lignocellulosics.

### BRIEF SUMMARY OF THE INVENTION

The current invention provides genetically modified bacterial strains that comprise genetic modifications to:

a) a gene encoding a secreted endoxylanase belonging to glycoside hydrolase family 10 or a homolog thereof (if present within the genome of the microorganism/bacterial strain), and genetic modifications to:

b) a gene encoding a secreted endoxylanase belonging to glycoside hydrolase family 11 or a homolog thereof (if present within the genome of the microorganism/bacterial strain), and/or

c) a gene encoding a secreted endoxylanase belonging to glycoside hydrolase family 30 or a homolog thereof (if present within the genome of the microorganism/bacterial strain),

wherein, said genetic modifications inactivate the enzymatic activity of the endoxylanases produced by said target gene.

The bacterial strains of the current invention may further comprise genetic modifications to one or more genes encoding proteins belonging to glycoside hydrolase family 43 (GH43), glycoside hydrolase family 8 (GH8), and/or glycoside hydrolase family 39 (GH39), and, optionally, modification to express and secrete alpha-glucuronidases of the GH67 and/or GH115 families. In certain embodiments of the invention, bacterial strains have “generally recognized as safe” (GRAS) status, for example, several B. subtilis strains (21, 22), can be used according to current invention.

The current invention also provides a method of producing XOS, AXOS, U-XOS, and U-AXOS, the method comprising:

a) culturing a bacterial strain in a culture medium comprising methylglucuronoxylans (MeGXₙ) and/or methylglucronoarabinoxylans (MeGAXₙ) under conditions that allow conversion of MeGXₙ and/or MeGAXₙ to XOS, AXOS, U-XOS, and/or U-AXOS by the bacterial strain, wherein the bacterial strain comprises genetic modifications as disclosed herein

and wherein, said genetic modifications inactivate the enzymatic activity of the endoxylanases produced by said target gene; and

b) optionally, purifying XOS, AXOS, U-XOS, and/or U-AXOS from the culture.

Even further, certain embodiments of the current invention provide nutraceutical compositions comprising XOS, AXOS, U-XOS, and/or U-AXOS produced according to the methods of current invention. Certain embodiments of the current invention provide nutraceutical compositions produced according to the methods of the current invention, the compositions comprising aldouronates, acidic xylooligosacchari des containing one or more methylglucuronate residues linked α-1,2 to xylose residues in the β-1,4-xylan backbone in methylglucuronoxylans. Additional embodiments provide precursors for the production of pentosan polysulfates and related oligosaccharides and polysaccharides with biological activities of glycosaminoglycans, which have pharmaceutical as well as nutraceutical applications.

### BRIEF DESCRIPTION OF THE DRAWINGS

FIG. 1. Scheme for the generation of XOS from MeGXₙ using GH10, GH11, and GH30 endoxylanases.

FIG. 2. Products generated from MeGXₙ by recombinant XynA, XynC, and both enzymes together. Purified recombinant XynA and XynC, 0.1 units of each in 100 μl reaction mixture, were incubated with 0.2% sweetgum MeGXₙ in 0.05 M sodium acetate buffer, pH 6.0 for 18 h. Samples (10 μl) were spotted on silica gel TLC plates, developed in solvent and detected as described in Materials and Methods. Standards of aldouronates (U-XOS) included 10 nmol each of MeGX₁, MeGX₂, MeGX₃, and MeGX₄. Standards of xylose and XOS included X₁ (10 nmol), X₂ (20 nmol) X₃ (10 nmol) and a trace amount of X₄ in the X₃ preparation.

FIGS. 3A-3C. Comparison by ¹H-NMR of products generated by recombinant XynA, XynC and the combination of both enzymes. Reaction mixtures containing 0.5% sweetgum MeGXₙ in 0.05 M sodium acetate buffer pH 6.5 and enzyme were incubated for 18 h at 37° C. and exchanged with D₂O. Samples representing a 3.0 ml reaction mixture containing 15 mg MeGXₙ were exchanged with D₂O through successive lyophylization steps, dissolved in 99.99% DA) to a final volume of 1.0 ml and analyzed on a Mercury 300 Spectrometer as described in the Methods section. The XynA digest contained 13.6 μmol of acetone to serve as an internal standard. The XynC and the combination of XynA and XynC digests contained 31.3 μmol of acetone. FIG. 3A) 0.1 units of recombinant XynA. FIG. 3B) 0.1 units of recombinant XynC. FIG. 3C) 0.1 units of recombinant XynA and 0.1 units of recombinant XynC.

FIG. 4. Growth comparisons of B. subtilis strain 168, MR42, MR44 and MR45 on MeGXₙ. For preparing inocula for growth comparisons, 18 h standing cultures (1.0 ml of LB with antibiotics: MR42 (kanamycin, 5 μg/ml), MR44 (spectinomycin, 100 μg/ml) and MR45 (kanamycin, 5 μg/ml, spectinomycin, 100 μg/ml), 0.03 ml were inoculated into 1.0 ml of the same medium without antibiotics and incubated for 3 h at 37° C. with shaking Cultures of these strains (Table 2) grown in LB medium to late log phase (OD600=0.6-0.7) were inoculated into 20 ml of Spizizen's minimal media with 0.5% SG MeGXₙ and 0.1% yeast extract without antibiotics to give an OD600 of 0.03. Cells were cultured at 37° C. with gyratory shaking (200 rpm).

FIG. 5. Accumulation of U-XOS by B. subtilis strains. Media (10 μl) from cultures of B. subtilis strains described for FIG. 4 were spotted on silica gel TLC plates, developed in solvent and detected as described in Materials and Methods. Standards of aldouronates (U-XOS) included 10 nmol each of MeGX₁, MeGX₂, MeGX₃, and MeGX₄. Standards of xylose and XOS included X₁ (10 nmol), X₂ (20 nmol) X₃ (10 nmol) and a trace amount of X₄ in the X₃ preparation.

FIGS. 6A-6B. MALDI-TOF MS analysis of products generated by recombinant GH30 XYNC and cultures of strain MR44 from MeGXₙ. Numbers are assigned to species based upon the number of xylose units appended to an aldouronate containing a single MeG and Na⁺ and/or K⁻ adducts as defined in Tables 4 and 5. FIG. 6A) Recombinant XynC (0.1 units) from B. subtilis strain 168 was incubated at 37° C. in 0.1 ml of 0.5% sweetgum MeGXₙ in 0.05 M sodium acetate buffer, pH 6.0 for 18 h. Samples were removed and processed for MS as described in the Materials and Methods section. As Na⁺ was the predominant cation in the reaction medium, the Na⁺ adduct was the prominent species detected. FIG. 6B) MR44 was cultured for 24 h as described in the legend for FIG. 4. Samples were removed and processed for MS as described in the Materials and Methods section. With K⁺ as the predominant cation in the medium, the K⁺ adduct was the prominent species detected. Numbers above the predominant adduct species represent the number of xylose residues in the U-XOS. Alpha-cyclodextrin (α-CD) was the internal standard used in all analyses.

FIGS. 7A-7C. ¹H-NMR analysis of U-XOS products accumulated in cultures. Samples from stationary phase cultures (3.0 ml of 20 ml culture at 25 h, FIG. 4) were centrifuged to remove cells. The cell-free medium was concentrated by lyophilization and exchanged with 99.9% D₂O with 3 successive treatments. After a final lyophilization the sample was dissolved in 99.9% D₂O to a volume of 1.00 ml to which was added 2.3 μl of 99.7% acetone (31.3 μmol) and analyzed on a 300 MHz Mercury 300 spectrometer as described in the Materials and Methods section. FIG. 7A) B. subtilis strain 168; FIG. 7B) MR42; FIG. 7C) MR44.

FIG. 8. Schematic for the release of X₁, X₂, and MeGX₃ in B. subtilis strain 168 GH11 XynA (lower arrows) and GH30 XynC (upper arrows) hydrolyzed MeGXₙ to produce X₁, X₃ and MeGX₃ and X₂ and X₃ were assimilated b B. subtilis strain 168. As X₃, X₂ were rapidly and X₁ slowly consumed , MeGX₃ accumulated in culture media.

FIG. 9. Scheme for MeGXₙ processing by B. subtilis strains. MeGX₄ or MEGX₄₋₁₂ were accumulated in the culture media of mutant strains, MR42 (ΔxynC) or MR44 (ΔxynA). B. subtilis strain 168 depolymerized MeGXₙ with secretion of XynA and XynC, assimilation and metabolism of X₃, X₂, and X₁, and MeGX₃ was accumulated in culture medium.

FIGS. 10-13. Accumulation of XOS for mutagenized B. subtilis strains. Strain 3 (FIG. 10), 5 (FIG. 11), 6 (FIG. 12), F3 (FIG. 13).

FIG. 14. Samples taken from stationary phase cultures were analyzed by TLC as shown in FIG. 14. Saccharides detected with N-(1-Naphthyl) ethylenediamine dihydrochloride staining showed the accumulation of xylobiose and xylotriose along with small quantities of xylose. This demonstrates the abilities of all 4 stains to accumulate neutral oligosaccharides from xylans as compared to medium and the non-mutagenized wild-type parent strain (B. subtilis 168).

FIG. 15. Inactivation of thrombin activity over the ranges tested indicated 50% inhibition for heparin at 0.21 mg/ml while sulfated MeGX oligo showed a 50% inhibition at 0.0056 mg/ml (see FIG. 15). Sulfated MeGXₙ polysaccharide showed no inhibition over the test range indicated below. On a weight basis sulfated oligosaccharides were 37.5 times more effective than heparin at inhibiting thrombin activation.

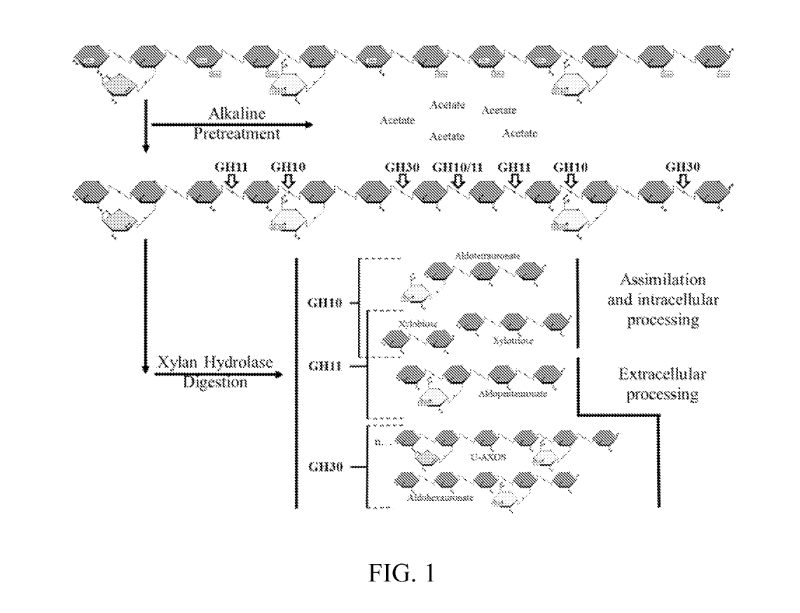

### BRIEF DESCRIPTION OF THE SEQUENCES

SEQ ID NO: 1 represents forward primer used for the amplification of xynD-xynC-bglC′ genes from B. subtilis strain 168.

SEQ ID NO: 2 represents reverse primer used for the amplification of xynD-xynC-bglC′ genes from B. subtilis strain 168.

SEQ ID NO: 3 represents forward primer used in the amplification of DNA containing xynA genes from B. subtilis strain 168.

SEQ ID NO: 4 represents forward primer used in the amplification of DNA containing xynA genes from B. subtilis strain 168.

SEQ ID NO: 5 represents forward primer used for the amplification of DNA containing GH11 endoxylanase xynA gene from B. subtilis strain 168.

SEQ ID NO: 6 represents reverse primer used for the amplification of DNA containing GH11 endoxylanase xynA gene from B. subtilis strain 168.

SEQ ID NO: 7—Bacillus subtilis strain 168 yxxF protein.

SEQ ID NO: 8—Bacillus subtilis strain 168 yxxF gene.

SEQ ID NO: 9—Bacillus subtilis strain 168 kinC protein.

SEQ ID NO: 10—Bacillus subtilis strain 168 kin C gene.

### DETAILED DISCLOSURE OF THE INVENTION

The current invention provides genetically modified microorganisms that comprise genetic modifications to one or any combination of:

a) a gene encoding a secreted endoxylanase belonging to glycoside hydrolase family 10 or a homolog thereof (if present within the genome of the microorganism/bacterial strain), and genetic modifications to:

b) a gene encoding a secreted endoxylanase belonging to glycoside hydrolase family 11 or a homolog thereof (if present within the genome of the microorganism/bacterial strain), and/or

c) a gene encoding a secreted endoxylanase belonging to glycoside hydrolase family 30 or a homolog thereof (if present within the genome of the microorganism/bacterial strain),

d) optional introduction of a gene encoding a secreted alpha-glucuronidase belonging to glycoside hydrolase family 67 or a homolog thereof,

e) optional introduction of a gene encoding a secreted alpha-glucuronidase belonging to glycoside hydrolase family 115 or a homolog thereof, and wherein, said genetic modifications inactivate the enzymatic activity of the endoxylanases produced by said target gene; and/or

optional inactivation of the kinC gene (or a homolog thereof) or xyyN gene (or a homolog thereof).

Table below summarizes the products that accumulate by culturing microorganisms having deletions of the genes according to the current invention (in the presence of methylglucuronoxylans (MeGXₙ, where n is the number of xylose residues), Table 1A, or methylglucuronoarabinoxylans (MeGAXₙ, where n is the number of xylose residues), Table 1B).

TABLE 1A
Products accumulated by culturing microorganisms
having deletions of xyn genes encoding GH11 or GH30
xylanases according to the current invention in the presence
of methylglucuronoxylans (MeGXₙ).

| | Glycoside | Glycoside | |
|---|---|---|---|
| Glycoside | Hydrolase | Hydrolase | |
| Hydrolase | Family 11, | Family 30, | |
| Family 10 | xynA | xynC | Products accumulated |
| − | + | + | MeGX₃ |
| − | − | + | MeGX₄₋₁₈ |
| − | + | − | MeGX₄ |

aldopentauronate methylglucuronoxylose compounds having 4-18 xylose residues (MeGX₄₋₁₈)
aldopentauronate methylglucuronoxylotetraose (MeGX₄),
aldopentauronate methylglucuronoxylotriose (MeGX₃)
xylotriose (X₃),
xylobiose (X₂),
xylose (X₁)

TABLE 1B
Products accumulated by culturing microorganisms having deletions
of some of the genes according to the current invention in the presence
of methylglucuronoarabinoxylans (MeGAXₙ).

| Glycoside | Glycoside | Glycoside | |
|---|---|---|---|
| Hydrolase | Hydrolase | Hydrolase | |
| Family 10 | Family 11 | Family 30 | Products formed |
| − | + | + | MeGX₃ |
| − | − | + | MeGX₄₋₁₈ |
| − | + | − | MeGX₄ |

aldopentauronate methylglucronoarabinoxylan compounds having 4-18 xylose and a variable number of arabinose residues(MeGAX₄₋₁₈),
methylglucronoarabinotetraxylan (MeGAX₄),
methylglucronoarabinotrixylan (MeGAX₃),
xylotriose (X₃),
xylobiose (X₂),
xylose (X₁)

Non-limiting examples of the microorganisms that can be modified according to the methods of current invention include bacteria, fungi, diatoms, cyanobacteria, yeast, etc.

A list of organisms that contain one or more of the secreted endoxylanases of glycoside hydrolase families 10, 11, 30, 8, 43, and 39 is provided in Table 7. Any of these organisms can be modified according to the teachings of the current invention.

Table 7 provides a list of organisms and alphanumeric codes indicating UniProtKB/Swiss-Prot Accession numbers of secreted endoxylanases of glycoside hydrolase families 10, 11, 30, 8, 43, and 39 present in those organisms. The genes encoding the disclosed endoxylanases can be readily identified by reference to the UniProtKB/Swiss-Prot Accession numbers (which provide the amino acid sequences of the endoxylanases) and readily inactivated according to methods known in the art or disclosed herein. A person of ordinary skill in the art can check a particular organism in the table and identify which of the secreted endoxylanases of glycoside hydrolase families 10, 11, 30, 8, 43, 67, 115 and 39 are present or absent in that organism. Based on this information, a skilled artisan can design strategies genetically modify the organism according to the teachings of the current invention (e.g., such that the microorganism is engineered to contain a secreted endoxylanase of glycoside hydrolase families 10, 11, 30, 8, 43 and/or 39 and, optionally, an alpha-glucuronidase of the GH67 and/or 115 family or such that organisms containing a secreted endoxylanase of glycoside hydrolase families 10, 11, 30, 8, 43 and/or 39 is inactivated in the genome of the microorganism). Such organisms and genetic modification strategies are within the purview of the current invention. For an organism not present in the list provided in Table 7, a skilled artisan can study the genomic data for the organism and identify which of the secreted endoxylanases of glycoside hydrolase families 10, 11, 30, 8, 67, 115, 43, and 39 are present or absent in that organism. Based on this information, a skilled artisan can design strategies genetically modify the organism according to the teachings of the current invention. Such organisms and genetic modification strategies are also within the purview of this invention.

The microorganisms of the current invention may further comprise genetic modifications to one or more genes encoding proteins belonging to glycoside hydrolase family 43 (GH43), glycoside hydrolase family 8 (GH8), and/or glycoside hydrolase family 39 (GH39).

In certain embodiments of the invention, bacteria or other microorganisms having the “generally recognized as safe” (GRAS) status, for example, several B. subtilis (21, 22), can be developed as biocatalysts for the production of U-XOS from MeGXₙ. Examples of GRAS microorganisms include, but are not limited to, Aspergillus niger, Aspergillus oryzae, Bacillus coagulans, Bacillus lentus, Bacillus lincheniformis, Bacillus pumilus, Bacillus subtilis (non-antibiotic producing strains only), Bacteroides amylophilus, Bacteroides capillosus, Bacteroides ruminocola, Lactobacillus cellobiosus, Lactobacillus curvatus, Lactobacillus delbruekii, Lactobacillus fermentum, Lactobacillus lactis, Lactobacillus plantarum, Bacteroides suis, Bifidobacterium adolescentis, Bifidobacterium animalis, Bifidobacterium bifidum, Bifidobacterium infantis, Bifidobacterium longum, Bifidobacterium thermophilum, Lactobacillus acidophilus, Lactobacillus brevis, Lactobacillus bulgaricus, Lactobacillus casei, Lactobacillus reuterii, Leuconostoc mesenteroides, Pediococcus acidilacticii, Pediococcus cerevisiae (damnosus), Pediococcus pentosaceus, Propionibacterium freudenreichii, Propionbacterium shermanii, Streptococcus cremoris, Streptococcus diacetylactis, Streptococcus faecium, Streptococcus intermedius, Streptococcus lactis, and Streptococcus thermophilus.

It is understood that certain organisms, for example, certain GRAS organisms, do not endogenously contain one or more of the secreted endoxylanases of glycoside hydrolase families 10, 11, and/or 30 within their genome or alpha-glucuronidases of the GH67 and/or GH115 families. Such organisms can be genetically modified to express one or more secreted endoxylanases of glycoside families 10, 11, and/or 30 and, optionally, or alpha-glucuronidases of the GH67 and/or GH115 families to practice the current invention (or in certain embodiments, have one or more of the secreted endoxylanase genes found within the genome of the microorganism deleted such that it produces a desired methylglucuronoxylan (MeGXₙ) or methylglucuronoarabinoxylans (MeGAXₙ) product. For example, an organism lacking secreted endoxylanases and alpha-glucuronidases of glycoside hydrolase families 10, 11, 67, 115 and 30 can be genetically modified to express secreted endoxylanases of glycoside hydrolase family 11 or 30 and, optionally, or alpha-glucuronidases of the GH67 and/or GH115 families to practice the current invention. An organism endogenously expressing a secreted endoxylanase of glycoside hydrolase family 10, but not expressing secreted endoxylanases of glycoside hydrolase families 11 and 30 can be genetically modified to delete the secreted endoxylanase of glycoside hydrolase family 10 and express secreted endoxylanase of glycoside hydrolase family 11 or 30 and, optionally, alpha-glucuronidases of the GH67 and/or GH115 families by genetic modifications of the organism. Thus, given the teachings of the current invention and based on various permutations and combinations of the genes involved, additional strategies of genetic modifications of organisms expressing or not expressing one or more secreted endoxylanases of glycoside hydrolase families 10, 11, and 30 and expressing, or not expression alpha-glucuronidases of the GH67 and/or GH115 families can be designed by a person of ordinary skill in the art. Such embodiments are within the purview of the current invention.

Further, one or more genes encoding one or more secreted endoxylanases of glycoside hydrolase family can be expressed in a host organism by a variety of methods, for example, by incorporation of the one or more genes in to the genome of the organism or expressing the one or more genes through a vector capable of driving expression of proteins encoded by the one or more genes. Additional methods of expressing one or more endogenous genes in a host organism are well known to a person of ordinary skill in the art and such embodiments are within the purview of the current invention.

Certain bacterial strains contain secreted endoxylanases of glycoside hydrolase family 10, 11, and 30. For example, Paenibacillus sp. JDR2 contains endoxylanases of glycoside hydrolase family 10 and 11 as summarized below:

1. GH10 (GenBank Accession Number: AJ938162); and/or

2. GH11 (GenBank Accession Number: ACT03278).

Paenibacillus sp. JDR2 can be genetically modified according to current invention to:

a) inactivate enzymatic activity of secreted endoxylanases of glycoside hydrolase family 10, and/or

b) inactivate enzymatic activity of secreted endoxylanases of glycoside hydrolase family 11 (e.g., Accession No. ACT03278.1).

Certain other bacterial strains can lack one or more genes encoding secreted endoxylanases belonging to GH10, GH11, and/or GH30. Such bacterial strains can be further modified to delete genes encoding certain secreted endoxylanases in order to produce a desired product. For example, B. subtilis strain 168 lacks a gene encoding a secreted protein belonging of glycoside hydrolase family 10. B. subtilis strain 168 can, thus, be genetically modified to inactivate secreted endoxylanase belonging to glycoside hydrolase family 11 or a homolog thereof, and/or inactivate a secreted endoxylanase belonging to glycoside hydrolase family 30 or a homolog thereof Accordingly, the current invention provides B. subtilis strain 168 comprising genetic modifications to:

a) a gene encoding a secreted endoxylanase belonging to glycoside hydrolase family 11 or a homolog thereof, and/or

b) a gene encoding a secreted endoxylanase belonging to glycoside hydrolase family 30 or a homolog thereof,

and wherein, said genetic modifications inactivate the enzymatic activity of the secreted endoxylanases produced by said target genes.

B. subtilis strain 168 having these genetic modifications can further comprise genetic modifications to one or more genes encoding proteins belonging to glycoside hydrolase family 43 (GH43), glycoside hydrolase family 8 (GH8), and/or glycoside hydrolase family 39 (GH39), wherein, said genetic modifications inactivate the enzymatic activity of proteins produced by those genes.

Genes encoding GH11 and/or genes encoding GH30 can be deleted in Bacillus subtilis strain 168 according to methods described herein under the Materials and Methods section. A person of ordinary skill in the art can design other strategies for deleting target genes in Bacillus subtilis or other organisms of interest (e.g., the GRAS strains discussed above) to arrive at the current invention and such strategies are within the purview of this invention.

For example, a person of ordinary skill in the art can identify a bacterial strain suitable for genetic modifications according to current invention. A bacterial strain expressing secreted endoxylanases belonging to families GH10, GH11, and GH30 can be genetically modified to delete secreted endoxylanases belonging GH10, and GH11 and/or GH30 to arrive at the current invention; whereas, a bacterial strain lacking secreted endoxylanase of family GH10 and expressing secreted endoxylanase of family GH11 and/or GH30 can be genetically modified to delete secreted endoxylanase of family GH11 and/or GH30 to arrive at the current invention. Further, a bacterial strain only expressing secreted endoxylanase of families GH11 and GH30 can be genetically modified to inactivate either or both secreted endoxylanases of families GH11 and GH30 to arrive at the current invention. Any of the aforementioned strains in this paragraph can, optionally, be genetically modified to express and secrete alpha-glucuronidases of the GH67 and/or GH115 families.

The genetically modified bacterial strains (such as Bacillus spp.) of the current invention, for example, bacterial strains having inactivated genes encoding secreted endoxylanases of family GH10, inactivated secreted endoxylanase of family 11, and/or inactivated secreted endoxylanase of family GH30; can be further genetically modified to inactivate one or more transporters involved in transfer of XOS, AXOS, U-XOS, and/or U-AXOS into the bacterial cell (for example, msmE (gene ID 646319609, locus tag BSU30270) encoding a sugar-binding protein and/or frlO (gene ID 646319875, locus tag BSU32600). Alternatively, certain strains can be genetically modified to inactivate the kinC gene (or a homolog thereof) and/or the yxxF gene (or a homolog thereof).

“Mutation” (and grammatical variants thereof) or “inactivation” (and grammatical variations thereof) refers to genetic modifications done to the gene including the open reading frame, upstream regulatory region and downstream regulatory region. The gene mutations result in a down regulation or complete inhibition of the transcription of the open reading frame (ORF) of the gene. Gene mutations can be achieved either by deleting the entire coding region of the gene (ORF) or a portion of the coding nucleotide sequence (ORF), by introducing a frame shift mutation within the coding region, by introducing a missense mutation, insertion of sequences that disrupt the activity of the protein encoded by the gene (e.g., via transposon mutagenesis), by introducing a stop codon or any combination of the aforementioned gene mutations. In one aspect, the mutation or inactivation of the genes in the chromosome of the microorganism is accomplished without introducing genes or portions thereof from exogenous sources (e.g., deletion of all or a portion of the ORF). Another aspect provides for the mutation of endogenous genes by the introduction of one or more point mutation(s) or by introducing one or more stop codon in the open reading frame of the endogenous gene that is being modified.

Genetically modified bacterial strains of the current invention, for example, strains of B. subtilis strain 168, can be used for the conversion of MeGXₙ and/or MeGAXₙ to release XOS, AXOS, U-XOS, and/or U-AXOS. The pathways for this conversion determine the efficiency with which B. subtilis strain 168, and other strains and species that have this GH11/GH30 system for xylan depolymerization, are able to convert a lignocellulosic resource to targeted products.

Accordingly, the current invention also provides a method of producing XOS, AXOS, U-XOS, and U-AXOS, the method comprising:

a) culturing a genetically modified microorganism, for example, a bacterial strain, in a culture medium comprising methylglucuronoxylans (MeGXₙ) and/or methylglucronoarabinoxylans (MeGAXₙ) under conditions that allow conversion of MeGXₙ and/or MeGAXₙ to XOS, AXOS, U-XOS, and/or U-AXOS by the microorganism, wherein the genetically modified microorganism comprises genetic modifications as disclosed herein; and

b) optionally, purifying XOS, AXOS, U-XOS, and/or U-AXOS from the culture. The current invention also provides a method of producing XOS, AXOS, U-XOS, and U-AXOS, the method comprising:

a) culturing a genetically modified B. subtilis strain 168 in a culture medium comprising methylglucuronoxylans (MeGXₙ) and/or methylglucronoarabinoxylans (MeGAXₙ) under conditions that allow conversion of MeGXₙ and/or MeGAXₙ to XOS, AXOS, U-XOS, and/or U-AXOS by the genetically modified B. subtilis strain 168 disclosed herein; and

b) optionally, purifying XOS, AXOS, U-XOS, and/or U-AXOS from the culture.

Furthermore, the current invention provides nutraceutical or pharmaceutical compositions comprising XOS, AXOS, U-XOS, and/or U-AXOS produced by the methods of the current invention. In an embodiment of the invention, the pharmaceutical composition of U-AXOS contains sulfated U-AXOS, for example, pentosan polysulfate. Certain embodiments of the current invention provide compositions comprising aldouronates, acidic xylooligosaccharides containing one or more methylglucuronate residues linked α-1,2 to xylose residues in the β-1,4-xylan backbone in methylglucuronoxylans. The compositions of the current invention can further comprise pharmaceutically acceptable carriers.

Purified U-AXOS can be further sulfated to produce pentosan polysulfate. PPS can be used in the treatment of interstitial cystitis in humans and osteoarthritis in horses. Novel properties of PPS are being discovered that are expected to extend the use of PPS for treatment of disease associated with mucopolysaccharodosis.

### Materials and Methods

B. subtilis Strains and Media

Bacillus subtilis subsp. subtilis strain 168 was obtained from the Bacillus Genetic Stock Center (see world-wide website: bgsc.org). B. subtilis strains were cultured in LB broth (Lennox L broth), low salt formula (RPI corp.) at 37° C. and Spizizen's medium (23) was used for cultivation on different carbohydrate substrates. Spizizen's medium contained the following composition per liter: K₂HPO₄ (14 g), KH₂PO₄ (6 g), Na₃C₆H₅O₇.2H₂O (1 g), 0.2% (NH₄)₂SO₄, 0.02% MgSO₄.7H₂O, and was supplemented with tryptophan at 25 μg/ml. Unless otherwise noted, 0.1% yeast extract (Difco) was included.

Construction of B. subtilis xylanase mutants, MR42 (168, ΔxynC-Km), MR44 (168, ΔxynA-Spc), and MR45 (168, ΔxynA-Spc, ΔxynC-Km)

For construction of a B. subtilis xynC GH30 xylanase mutant strain, the 4,270 bp DNA fragment containing xynD-xynC-bglC′ genes was amplified using B. subtilis strain 168 genomic DNA as the template and bg-BS0104F (GCATACCTCGAGCGTCTGGCAATGGCGGTGTA, SEQ ID NO: 1), and bg-BS0104R (AGCAGCAGCAATCTACAACCT, SEQ ID NO: 2) as the primers. The amplified product was ligated into plasmid vector pUC19 hydrolyzed by HinCII (pMSR450). The kanamycin resistant gene (Km) fragment (1,486 bp) was prepared from plasmid pMSP3535VA after hydrolysis by ClaI and filling-in using DNA polymerase I, Klenow fragment (Klenow). A 1,235 bp fragment of xynC was removed from the plasmid pMSR450 after hydrolysis by AflII and filling in the ends with Klenow, and the km fragment was inserted at this location (pMSR451). A 4,527 bp of xynD-km-bglC′ fragment was amplified by PCR and introduced into B. subtilis strain 168 according to the procedure described by Rhee et al. (24). Transformants were selected using LB-agar medium with 5 μg/ml kanamycin. Disruption of the xynC gene in the MR42 mutant was confirmed by PCR amplification.

In order to construct the B. subtilis xynA GH11 xylanase mutant, the 1,935 bp DNA fragment containing the xynA gene of B. subtilis strain 168 was amplified using the primers, xA-BS0204F (GGAGTGCTCGAGAGGAGGAAGTCATGGTAAGC, SEQ ID NO: 3), and xA-BS0204R (GCGTTGTCTAGATCGTAGAGTCCCCATTCATAAAT, SEQ ID NO: 4). The PCR product was ligated into plasmid vector pUC19 hydrolyzed by HinCII (pMSR452). A 519 bp fragment was removed from the middle of the xynA gene in plasmid pMSR452 after hydrolysis by NheI and EcoRV and the NheI end was filled in using the Klenow treatment. The spectinomycin resistant gene (Spc) fragment (1,411 bp) from pAW016 was ligated into this region to yield plasmid pMSR453. A PCR product of 2,831 bp containing xynA interrupted with the spc resistant gene was introduced into B. subtilis strain 168 and MR42. Transformants were selected using LB-agar medium containing spectinomycin (100 μg/ml). Disruption of the xynA gene in the MR44 and MR45 mutants were confirmed by PCR amplification.

TABLE 2
Bacterial strains, and plasmids used in this study

| Strains and | | |
|---|---|---|
| Plasmids | Relevant genotype | References |
| Bacillus subtilis | | |
| 168 | trpC2 | |
| MR42 | 168, ΔxynC-Km | This study |
| MR44 | 168, ΔxynA-Spc | This study |
| MR45 | 168, ΔxynA-Spc ΔxynC-Km | This study |
| Plasmids | | |
| pUC19 | | |
| pMSP3535VA | pVA380-1 and ColE1 | (43) |
| | replicons nisRK P_{nisA} Km^{r} | |
| pAW016 | Mini-Tn10 delivering vector | (44) |
| pMSR450 | pU19, xynD-xynC-bglC′ | This study |
| pMSR451 | pMSR450, Km^{r} | This study |
| pMSR452 | pUC19, xynA | This study |
| pMSR453 | pMSR452, Spc^{r} | This study |
| pLSW3 | pET15b, xynA | This study |

Preparation of GH11 and GH30 Endoxylanases from B. subtilis

For purification of GH11 endoxylanase XynA, the xynA gene was amplified by PCR with B. subtilis strain 168 genomic DNA as template and xynAF (ATGTCCCTCGAGAGCACAGACTACTGGCAAAATT, SEQ ID NO: 5) and xynAR (CGATAAGGATCCCCTACCTCCAGCAATTCCAA, SEQ ID NO: 6) as the primers. The amplified product (721 bp) hydrolyzed by XhoI and BamHI was ligated into plasmid pET15b, also hydrolyzed by XhoI and BamHI, yielding the plasmid pLSW3. E. coli Rosetta 2 cells were transformed with the ligation product and transformants were selected on LB containing ampicillin and chloramphenicol. The Rosetta 2 strain containing pLSW3 was cultured in a 500 ml of LB containing ampicillin and chloramphenicol in a 2.8-liter Fernbach flask at 37° C. with shaking at 250 rpm. When the optical density at 600 nm (Beckman DU640 spectrophotometer) reached 0.8, isopropyl β-D-1-thiogalactopyranoside (IPTG, 0.1 mM) was added to the culture to induce the T7 RNA polymerase. After 4 h of incubation at room temperature with shaking, cells were harvested by centrifugation (10,000×g, 10 min, 4° C.), washed twice with 25 ml of 20 mM sodium phosphate (pH 7.4), and resuspended in 20 ml of the same buffer. Cells were passed through a French pressure cell at 16,000 lb/in². The crude extract was clarified by centrifugation (30,000×g, 45 min, 4° C.), and the supernatant was filtered through a 0.22-μm filter and loaded onto a HiTrap HP chelating column (5 ml; GE Life Sciences) preconditioned with 0.1 M NiSO₄. Unbounded material was removed by washing with 10 column volumes of phosphate buffer containing 0.5 M NaCl (elution buffer), followed by 10 column volumes of elution buffer containing 50 mM imidazole, His-tagged XynA protein was eluted with 0.5 M imidazole in elution buffer. Imidazole was removed from the sample using a PD-10 column (GE Life Sciences) and protein eluted with 50 mM sodium acetate, pH 6.0. The activity of this XYNA enzyme was 44 Umg⁻¹. The GH30 endoxylanase XynC enzyme was prepared as a pure recombinant enzyme, 47 U mg⁻¹, as previously described (15, 16). One unit is the activity that generates 1 μmol reducing terminus per min at 30° C.

Preparation of Substrates and Analyses of Enzymes

Methylglucuronoxylan (MeGXₙ) was purified from sweetgum wood as previously described (14, 25). The preparations were analyzed for total carbohydrate (26), total uronic acid (27) and total reducing sugar (28). The average degree of polymerization (DP) (ratio of total carbohydrate to total reducing sugar) of these preparations was estimated to average 330. Xylanase assays were routinely performed using the reducing sugar assay with methylglucuronoxylan (MeGXₙ) as substrate (14). In some cases the multi-well plate BCA assay was used as described (29). Products generated from enzyme assay were identified following resolution by TLC.

Chromatographic (TLC) Analysis of Xylan Utilization

Samples were spotted onto 20 cm by 20 cm Silica gel 60 TLC plates (Millipore). Reaction products were separated by ascension with 150 ml of solvent (chloroform:acetic acid:water; 6:7:1; v:v:v) (30) allowing the solvent to migrate to within 1 cm of the top of the plate. Plates were allowed to dry prior to a second ascension. Plates were allowed to dry at ambient temperature overnight in a fume hood, sprayed with a solution containing 100 ml of methanol with 0.1685 g of N-(1-Naphthyl) ethylenediamine dihydrochloride and 3 ml of H₂SO₄, and heated at 100° C. to reveal resolved components.

Preparation and Analyses of Oligosaccharides

Digestions and analyses of products of MeGXₙ depolymerization with XynA and XynCwere carried out as previously described for XYNC from B. subtilis strain 168 (16). Cultures with 0.2%, 0.5%, or 1.0% MeGXₙ as the carbon source in modified Spizizen's medium containing 0.1% yeast extract were incubated at 37° C. with gyratory shaking (200 rpm) for 25 h. Samples of the cultures were directly spotted onto TLC plates for the identification of accumulated oligosaccharides as described above. Cells were removed by centrifugation (10,000 x g, 10 min, 4° C.), and the supernatants analyzed by MALDI-TOF MS and ¹H-NMR as described in detail below.

MALDI-TOF MS Analysis of MeGXₙ Hydrolysis Products

Products generated from the digestion of 0.2% MeGXₙ by recombinant XynA and/or recombinant XynC, and 0.5% MeGXₙ by B. subtilis strains 168, MR42 and MR44 were analyzed without further concentration by MALDI-TOF MS. Analysis of samples was performed on an Applied Biosystems Inc. Voyager-STR-DE operating in the positive-ion reflector mode with a delayed extraction time of 800 ns and a 20 kV accelerating voltage. Sufficient laser energy was employed to allow ionization, and 300-500 spectra were accumulated and averaged for each run. A stock matrix solution was prepared by dissolving 10 mg of 2,5-dihydroxybenzoic acid in 1 ml of 30% acetonitrile containing 0.1% trifluoroacetic acid (MeCN-TFA). A working matrix solution was prepared by mixing 29 μl of the stock matrix solution with 1 μl of 2 mg/ml a-cyclodextrin (MW=972.86 g/mole) in MeCN-TFA. For analysis, 3.33 μl of sample was added to 30 μl of MeCN-TFA. This was added to a microfuge tube containing 5-10 mg of Poros HS-20 strong cation exchanger that had been previously washed by suspension in MeCN-TFA and centrifugation. Samples were thoroughly mixed and centrifuged for 2 minutes to pellet the Poros resin. Resulting desalted supernatant aliquots of 1 μl were applied to the MALDI plate, followed by the addition of 1 μl of working matrix solution containing the internal standard a-cyclodextrin (MW=972.86 g/mole). The drops were mixed with a pipette and allowed to dry at room temperature prior to loading into the instrument.

NMR Analysis of MeGXₙ Hydrolysis Products

Samples for ¹H-NMR were prepared as previously described (16). This involved three successive dissolutions in 3 ml 99.9 atom percent D₂O (Sigma-Aldrich), each followed by lyophilization. Exchanged samples were dissolved to a concentration of 15 mg/ml total carbohydrate in 99.99% D₂O. To 1.0 ml of these preparations, 2.3 μl (31.3 μmol) of acetone was added as reference (2.225 ppm) and the final samples transferred to Wilmad 505-PS NMR tubes (Wilmad, Buena, N.J.). ¹H-NMR data collection was performed using a Mercury 300 MHz spectrometer with a 5 mm PFG Broadband probe at the Department of Chemistry, University of Florida (acquisition time=1.5 s; relaxation delay=2 s; number of scans=32). NMR data were analyzed and images were prepared using MestReNova (Mestrelab Research, Chemistry Software Solutions). Assignment of shift positions for specific atoms was based upon the studies of U-XOS derived methylglucuroxylans of Rudbekia fulgida by partial acid (TFA) hydrolysis (31).

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

### Products Generated by XYNA, XYNC, and Combinations

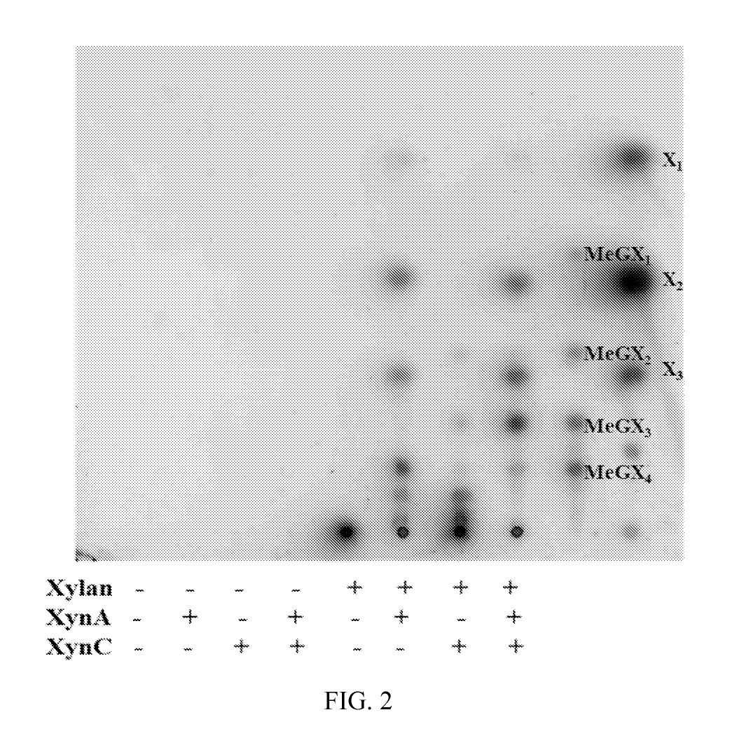

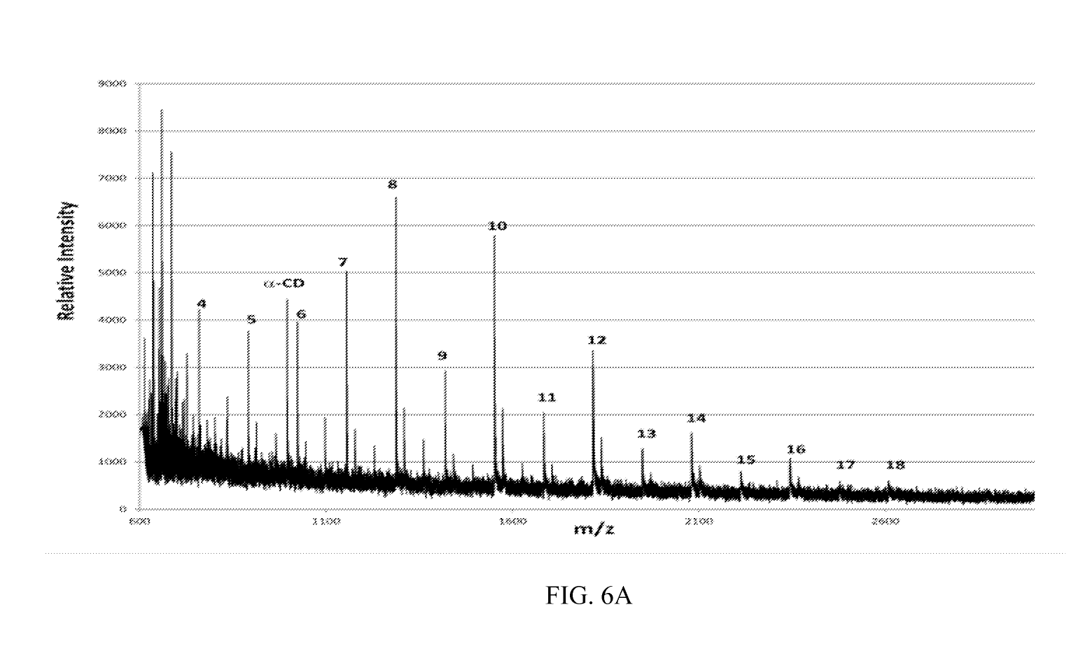

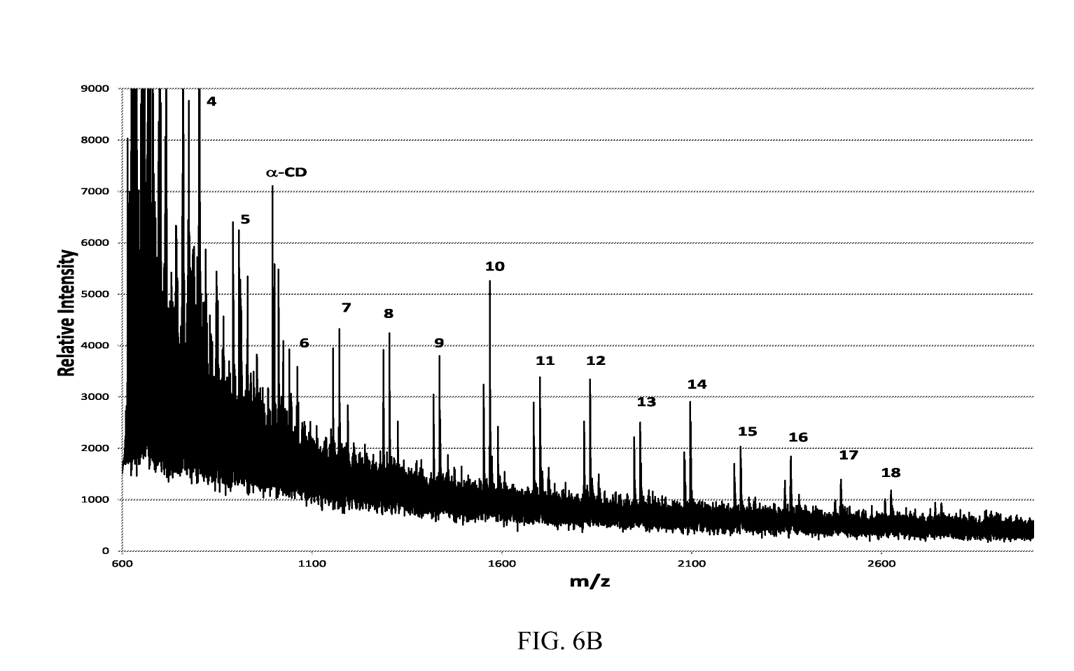

The products generated from MeGXₙ by equivalent activity units of recombinant XynA, XynC, and a combination of XynA and XynC enzymes, were resolved by TLC (FIG. 2). As expected from previous studies, XynA generates X₂, X₃ and the aldouronate MeGX₄ as the predominant products. Aldouronates of larger size, presumably MeGX₅ and MeGX₆, are also present in lesser concentrations and would likely be processed further to release more X₂ and free xylose. XynC generates a mixture of larger oligosaccharides that correspond to MeGX₄, MeGX₃, and MeGX₂ by TLC, with no detectable X₁, X₂ or X₃. The combination of XynA and XynC generate predominantly X₂ and X₃ for rapid assimilation and growth by B. subtilis cultures, with MeGX₃ as a predominant limit product. As shown in FIG. 2, XynC generates small amounts of products that correspond to MeGX₄, MeGX₃, and MeGX₂ with respect to mobility determined by TLC, with most products (estimated greater than 95%) larger than MeGX₄. MALDI-TOF MS analysis has identified a range of U-XOS from MeGX₂₋₁₈ for the products generated from sweetgum MeGXₙ in this study (FIG. 6A).

Evaluation of Products by ¹H-NMR

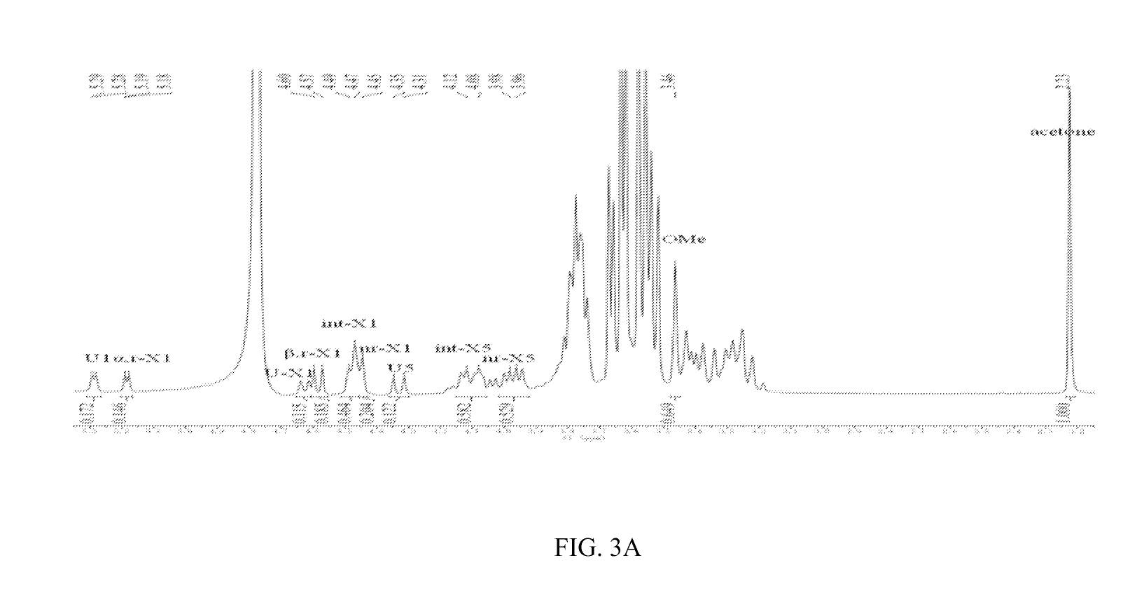

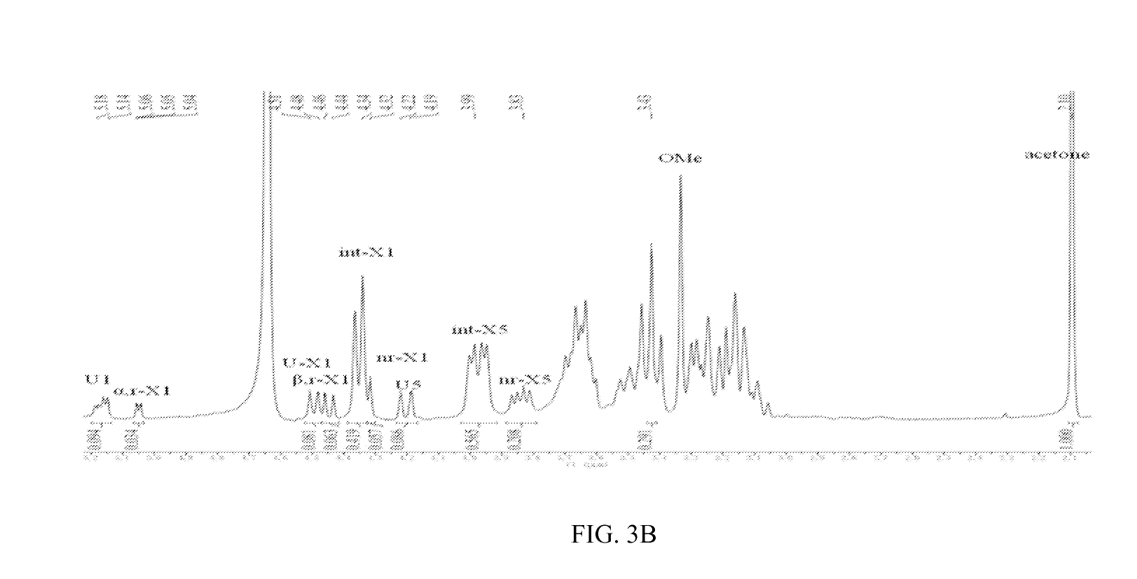

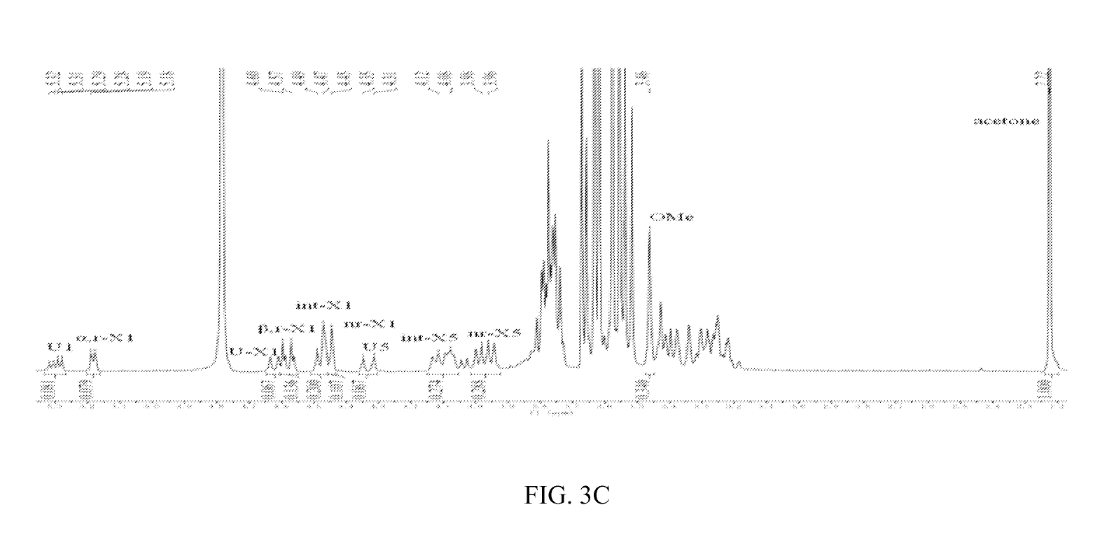

The products generated from sweetgum MeGXₙ by recombinant XynA, XynC and the combination of both enzymes were analyzed by ¹H-NMR. The products generated by the GH11 enzyme, XynA, provide a ¹H NMR spectrum (FIG. 3A) that includes limit product aldouronates, X₃, X₂, and a small amount of xylose (FIG. 2). Xylose ¹H—C's in the aldouronate cannot be quantitatively assigned. The ¹H linked to uronate Cl shows a single doublet at 5.27-5.33 ppm, characteristic for ¹H on Cl of MeG residues linked α-1,2 to xylose residues in oligosaccharides generated from methylglucuronoxylans by acid hydrolysis (31).

Products generated by the GH30 enzyme, XynC (FIG. 3B), include aldouronate limit products with no detectable xylose, X₂ or X₃ (FIG. 2). This provides a defining ¹H-NMR spectrum with signals from 4.32-4.34 ppm for ¹H atoms linked to the C5 of MeG (U5), from 4.08-4.14 ppm for ¹H atoms linked to the C5(X5) of internal β-1,4-linked (including the reducing terminal) xylose, and from 3.95-3.98 ppm for ¹H atoms linked to the C5(X5) of the non-reducing terminal xylose. The ratio of the ¹H integrals (int-X1+nr-X1+U-X1+u,y-X1+β,γ-X1)/U1 is 6.9, representing the average degree of substitution of xylose residues with MeG in the polymeric MeGXₙ. The ratio of integrals (int-X1+nr-X1+U-X1+β,γ-X1+α,γ-X1)/(β,γ-X1+α,γ-X1) is 6.7. Together, these values confirm that that each U-XOS bears a single MeG substitution. The ¹H atoms in the common molecular environment of the C4-linked —OCH₃ of the MeG residue show a prominent signal at 3.46 ppm, readily detectable in polymeric MeGXₙ as well as oligosaccharides (31). For all of the digests, the integration of ¹H-U5 is assigned a value of 1 for comparison with other hydrogens. The ratio of ¹H-U-OCH₃:¹H-U5 is 3.66:1, or 1.22:1 on a single hydrogen basis. These results support previous ¹³C-NMR studies of sweetgum MeGXₙ that found the U1, U4, and U-OCH₃ carbons are equivalent by integration, indicating all of the C4 carbons on the glucuronate residues contain —OCH₃ groups (32). The signal for the ¹H on Cl of the MeG shows a split doublet at 5.27-5.32 ppm, compared to the single doublet at 5.27-5.30 ppm for the products generated by XynA. The splitting of this doublet is characteristic for the ¹H on a uronate residue linked to the a xylose penultimate to the reducing terminal xylose in the oligosaccharide, whereby the 60:40 anomeric equilibrium of the α and β forms of the reducing terminal xylose influences the environment of the ¹H on Cl of the MeG that is α-1,2 linked to xylose adjacent to the reducing terminal residue (16, 33).

Products generated by the combination of the XynA and XynC enzymes shows a complex spectrum (FIG. 3C) that reflects, as in the case of the spectrum for the XynA digest (FIG. 3A), the presence of X₃, X₂ and xylose, as well as the aldouronate MeGX₃ (FIG. 2). The ¹H-U1 signal shows a split doublet at 5.27-5.32 ppm characteristic of substitution at a xylose penultimate to the reducing terminal xylose. The 60:40 ratio for this split supports a structure for the MeGX₃ generated by the action of XynC on the MeGX₄ generated by XynA as seen with the processing of birchwood xylan (34). The combination of XynA processing of the products generated by XynC, and of XynC processing of the products generated by XynA, is then responsible for the conversion of MeGXₙ to X₃, X₂ and xylose, as well as MeGX₃, in which a xylose flanked by xylose residues is substituted with an α-1,2-linked 4-O-methylglucuronate.

### EXAMPLE 2

### Effects of Deletion of Genes Encoding xynA and/or xynC on the Utilization of MEGX_{N} by B. subtilis

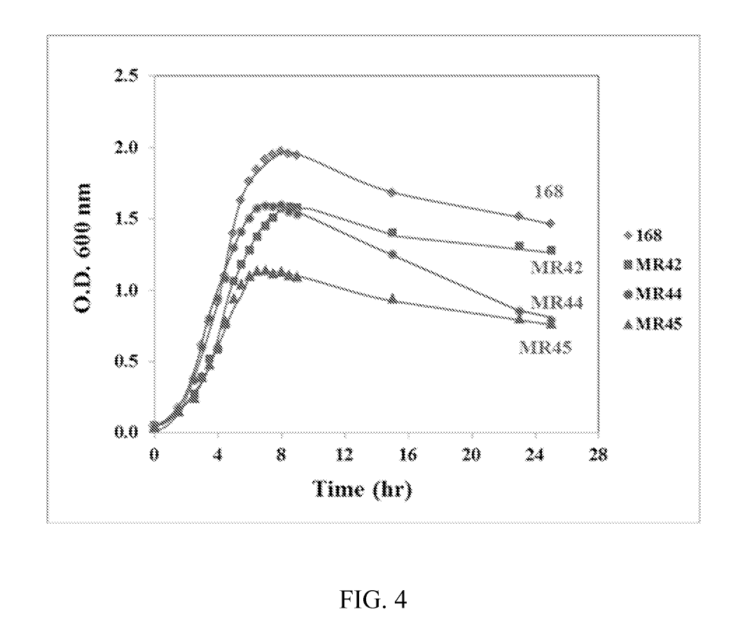

To test the role the XynA (Genbank Accession Number: AAA22897.1) and XynC (NCBI Reference Sequence: NP_389697.1) xylanases play in MeGX utilization, the genes encoding these enzymes were deleted individually to provide MR42 (ΔxynC) and MR44 (ΔxynA) or in combination to provide MR45 (ΔxynA, ΔxynC). The growth of these strains was compared to the parent strain B. subtilis strain 168 with 0.5% sweetgum MeGXₙ in a medium supplemented with yeast extract (FIG. 4).

Growth of the MR45 strain, which is unable to produce both XynA and XynC, was markedly lower than the parent 168 strain, reflecting the inability to generate xylotriose and xylobiose for growth. The MR44 strain, which secretes XynC but lacks XynA, initially grows to a higher turbidity than MR45 then drops to a level seen for MR45. This result, which was repeated, was surprising as the XynC enzyme does not generate detectable quantities of xylotriose, xylobiose or even xylose from MeGXₙ (FIG. 2). The MR42 strain that secretes XynA, but lacks XynC, is able to grow to a greater extent than MR44 as it does generate xylotriose and xylobiose from MeGXₙ.

TABLE 3
Utilization of carbohydrate during growth

| Time | Total carbohydrate (mM xylose equivalents) | | | |
|---|---|---|---|---|
| (h) | 168 | MR42 | MR44 | MR45 |
| 0 | 30.05 ± 2.62 | 30.05 ± 2.62 | 30.05 ± 2.62 | 30.05 ± 2.62 |
| 5 | 15.60 ± 1.38 | 22.10 ± 1.22 | 19.22 ± 0.94 | 24.70 ± 2.20 |
| 8 | 14.15 ± 0.82 | 18.96 ± 0.28 | 16.36 ± 0.87 | 21.20 ± 1.17 |
| 25 | 8.37 ± 1.08 | 13.27 ± 0.34 | 14.52 ± 0.54 | 21.11 ± 1.79 |

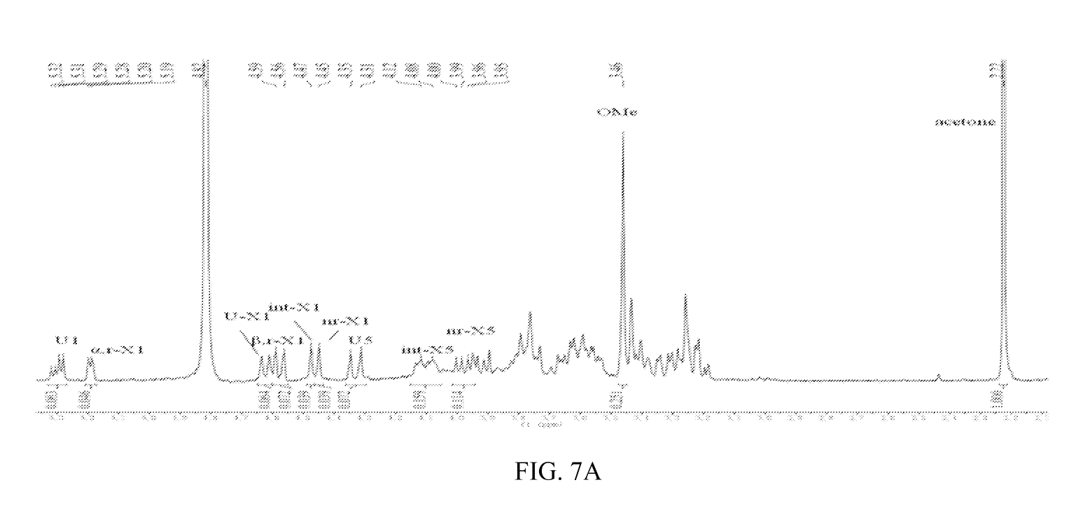

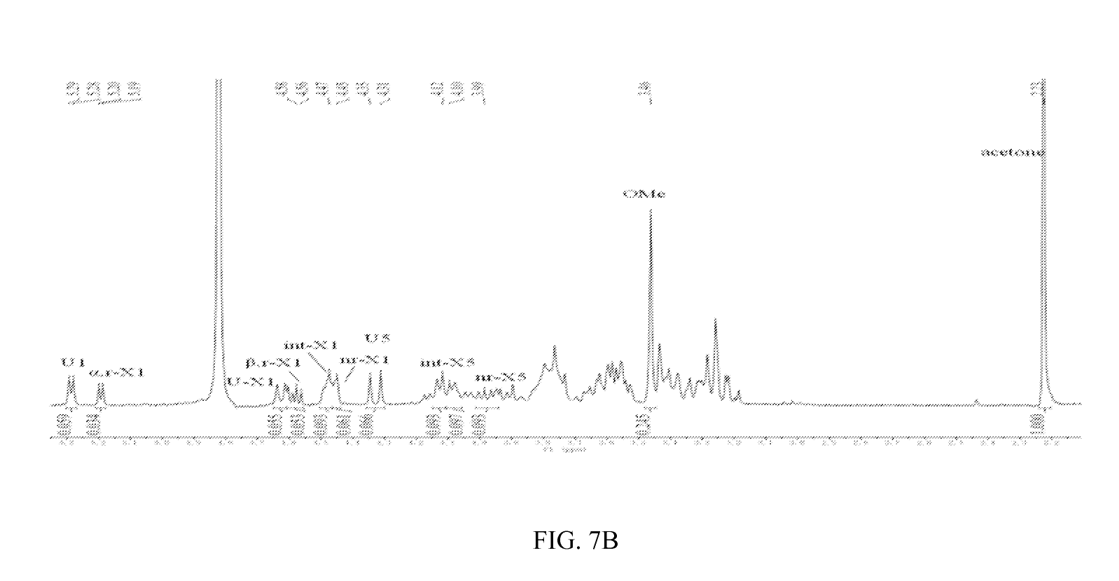

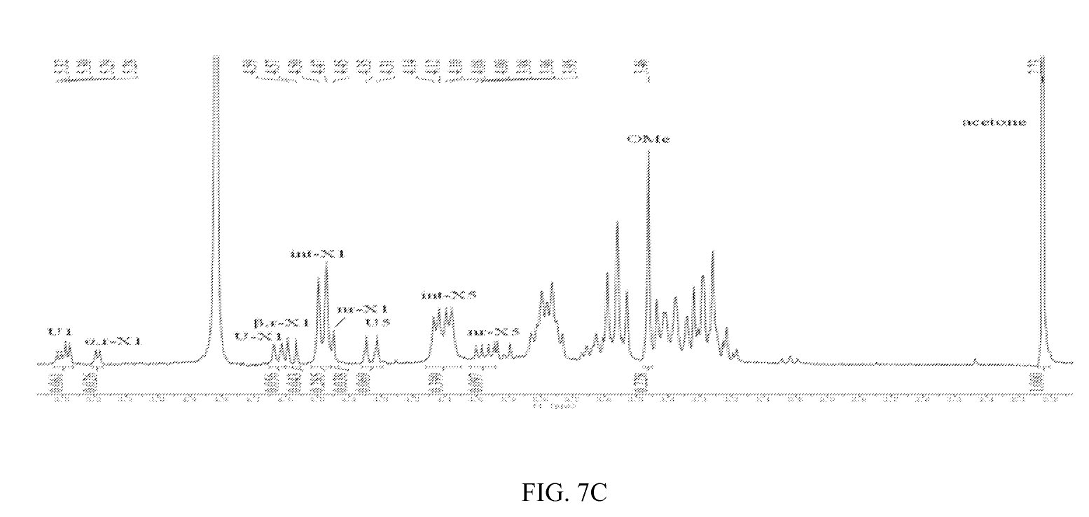

The utilization of carbohydrate (Table 3) was as expected greatest for B. subtilis strain 168 but incomplete with 28% remaining at 25 h, 17 h past the time of maximal growth during which 53% of the total carbohydrate had been consumed. During 8 h of exponential growth the MR45 strain lacking both XynA and XynC xylanases utilized 30% of the MeGXₙ. The absence of both xylose and XOS, detectable by TLC (FIG. 3), suggests the possibility that other sugars may provide some carbohydrate that do not depend upon xylanolytic depolymerization. Based upon analysis of sugar composition in hydrolysates of sweetgum lignocelluloses, glucans may comprise a small amount of the hemicel ulsose (xylan) fraction of sweetgum, although these were not detected as significant components upon NMR analyses of the polymeric MeGXₙ. Strain MR42, which secretes XynA, consumed 57% of the total carbohydrate, expected with the generation of X₂ and X₃, which are readily consumed. It is surprising that MR44 which secretes the GH30 (XynC) enzyme, shows 54% consumption of the MeGXₙ substrate as the aldouronate products of XynC digestion are not directly utilized. There may be exoxylanolytic activities that can process XynC products to release xylose and or XOS that support some growth. However the ¹H-NMR spectra of MR44 medium (FIG. 7C, Table 6) indicate xylose and MeG were present in a ratio similar to that found in the MeGXₙ which indicates nearly complete conversion of the MeGXₙ substrate to U-XOS products and their accumulation, a result which fits the established model of MeGXₙ processing by this enzyme.

Accumulation of U-XOS by B. subtilis Strains

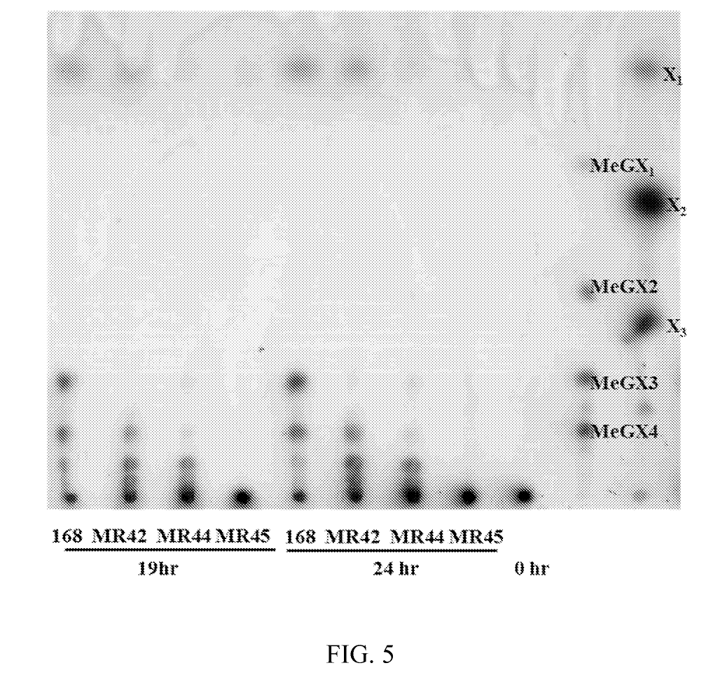

The culture media from each strain were evaluated for the accumulation of oligosaccharides by thin layer chromatography (FIG. 5). B. subtilis strain 168 shows the accumulation of MeGX₄ which is an expected product of the recombinant XynA and also MeGX₃ which is an expected product of the combination of recombinant XynA and XynC (FIG. 2). The appearance of some xylose was observed following digestion of MeGXₙ by XynA or a combination of XynA and XynC. Strain MR42 (ΔxynC) shows the accumulation MeGX₄, the expected product of XynA, as well as larger oligosaccharides with mobilities expected for MeGX₅ and MeGX₆. A similar mixture is noted in the XynA generated digest of MeGXₙ (FIG. 2). The much lower levels of these larger aldouronates in the medium from B. subtilis strain 168 cultures indicates the synergistic role XynA and XynC play in maximizing production of xylose and XOS for assimilation and growth. The MR44 (ΔxynA) strain accumulates MeGX₄₋₁₈ (FIG. 6B) and also traces of aldouronates with mobilities corresponding to MeGX₄, MeGX₃, and MeGX₂. The absence of detectable xylose indicates this strain and other strains lack an extracellular β-xylosidase that significantly participates in the processing of XOS generated. The MR45 (ΔxynA, ΔxynC) strain accumulates no detectable XOS, indicating XynA and XynC are the only endoxylanases secreted by B. subtilis.

The size of the oligosaccharides generated by the recombinant XynC allows the analysis of the medium of the MR44 culture by MALDI-TOF MS to determine the role of XynC in the accumulation of aldouronates that are not assimilated and metabolized. FIG. 6A shows the products generated by in vitro reaction with the recombinant XynC on the MeGXₙ used in the medium for the MR44 culture. The XynC generated aldouronate products with m/z corresponding to the sodium salts of MeGX₄ to MeGX₁₈ are similar to those previously documented (16). FIG. 6B shows the products accumulated by MR44 in the medium, with an M/z profile qualitatively similar to that observed for products generated by recombinant XynC in vitro in vitro. The m/z assignments are defined in Tables 4 and 5.

TABLE 4
MALDI-TOF MS Peak Assignments

| | | | Calculated | |
|---|---|---|---|---|
| | | Calculated | Mass of | Calculated |
| | Peak | Mass of | Na⁺ | Mass of K⁺ |
| Oligoxyloside | Label | Oliogoxyloside | Adduct | Adduct |
| MeGX4 | 4 | 735.68 | 758.67 | 774.77 |
| MeGX5 | 5 | 867.81 | 890.80 | 906.90 |
| MeGX6 | 6 | 999.94 | 1022.93 | 1039.03 |
| MeGX7 | 7 | 1132.07 | 1155.06 | 1171.16 |
| MeGX8 | 8 | 1264.20 | 1287.19 | 1303.29 |
| MeGX9 | 9 | 1396.33 | 1419.32 | 1435.42 |
| MeGX10 | 10 | 1528.46 | 1551.45 | 1567.55 |
| MeGX11 | 11 | 1660.59 | 1683.58 | 1699.68 |
| MeGX12 | 12 | 1792.72 | 1815.71 | 1831.81 |
| MeGX13 | 13 | 1924.85 | 1947.84 | 1963.94 |
| MeGX14 | 14 | 2056.98 | 2079.97 | 2096.07 |
| MeGX15 | 15 | 2189.11 | 2212.10 | 2228.20 |
| MeGX16 | 16 | 2321.24 | 2344.23 | 2360.33 |
| MeGX17 | 17 | 2453.37 | 2476.36 | 2492.46 |
| MeGX18 | 18 | 2585.50 | 2608.49 | 2624.59 |

TABLE 5
MALDI-TOF MS Peak Assignments

| | | Observed | Observed | | |
|---|---|---|---|---|---|
| | | Mass in | Mass in | Calculated | Calculated |
| | | Recombinant | MR44 | Mass of | Mass of |
| | Peak | XYNC | Culture | Na⁺ | K⁺ |
| Oligoxyloside | Label | Reaction | Medium | Adduct | Adduct |
| MeGX4 | 4 | 759.7 | 759.6 | 758.67 | 774.77 |
| MeGX5 | 5 | 891.8 | 891.8 | 890.80 | 906.90 |
| MeGX6 | 6 | 1023.8 | 1023.8 | 1022.93 | 1039.03 |
| MeGX7 | 7 | 1155.9 | 1171.9 | 1155.06 | 1171.16 |
| MeGX8 | 8 | 1285.0 | 1304.0 | 1287.19 | 1303.29 |
| MeGX9 | 9 | 1420.1 | 1436.0 | 1419.32 | 1435.42 |
| MeGX10 | 10 | 1562.1 | 1568.2 | 1551.45 | 1567.55 |
| MeGX11 | 11 | 1684.2 | 1700.2 | 1683.58 | 1699.68 |
| MeGX12 | 12 | 1816.3 | 1832.3 | 1815.71 | 1831.81 |
| MeGX13 | 13 | 1949.3 | 1964.3 | 1947.84 | 1963.94 |
| MeGX14 | 14 | 2080.4 | 2096.4 | 2079.97 | 2096.07 |
| MeGX15 | 15 | 2212.6 | 2228.4 | 2212.10 | 2228.20 |
| MeGX16 | 16 | 2345.5 | 2361.5 | 2344.23 | 2360.33 |
| MeGX17 | 17 | 2478.4 | 2493.5 | 2476.36 | 2492.46 |
| MeGX18 | 18 | 2608.4 | 2625.5 | 2608.49 | 2624.59 |

¹H-NMR Analysis of U-XOS Products Accumulated in Cultures

To identify the products accumulating in the media of the unmodified B. subtilis strain 168 as well as the MR42 (ΔxynC) and MR44 (ΔxynA) strains, cultures were grown to stationary phase and the media analyzed for accumulated products by ¹H-NMR. B. subtilis strain 168 shows the accumulation of MeGX₃ as the most prominent aldouronate along with products with TLC mobilities corresponding to MeGX₄ and MeGX₅ as well as small amounts of xylose (FIG. 5). Both X₂ and X₃ were prominent products in digestion of MeGXₙ by a combination of recombinant XynA and XynC (FIG. 3). These products would have been formed and consumed by B. subtilis strain 168, which secretes both of these enzymes. The clean spectrum for the medium for B. subtilis strain 168 allows for the identification of accumulated products. The integration of the ¹H signals for B. subtilis strain 168 (FIG. 7A) provides a semi-quantitative estimate of product amount of products and the extent of conversion of MeGXₙ that led to these products (Table 6). The ¹H-NMR spectra of MR44 medium (FIG. 7, Table 6) indicate MeG and xylose were present in a ratio similar to that found in the XYNC digest of MeGXₙ, indicating optimal conversion without further processing of the MeGXₙ substrate to U-XOS products by the MR44 strain.

TABLE 6
Quantitation of MeG and xylose accumulation in cultures by ¹H-NMR

| culture | U1 mM^{a} | X1 mM^{b} | X1/U1 | U5 mM^{c} | X5 mM^{d} | X5/U5 | % MeGXₙ^{e} |
|---|---|---|---|---|---|---|---|
| 168 | 11.8 | 48.9 | 4.1 | 13.5 | 51.3 | 3.8 | 88% |
| MR42 | 11.1 | 55.6 | 5.0 | 12.4 | 53.2 | 4.3 | 83% |
| MR44 | 11.5 | 84.2 | 7.3 | 13.0 | 93.2 | 7.1 | 86% |

^{a}¹H-U1 determined as the integration ratio of ¹H atom equivalents of MeG ¹H-C1 (5.28-5.32 ppm) to acetone ¹H—(CH₃)₂ (2.23 ppm) set to 1.00 for 188 mM ¹H atom equivalents.
^{b}¹H-X1 determined as the ratio of the sum of ¹H integrations for α,γ-X1 (5.19-5.20 ppm), U-X1 (4.60-4.68 ppm), β,γ-X1 (4.56-4.59 ppm), int-X1 (4.47-4.5 ppm), and nr-X1 (4.45 ppm) to 1.00 for acetone at 188 mM ¹H atom equivalents.
^{c}¹H-U5 determined as the ratio of ¹H integration (4.31-4.35 ppm) to 1.00 for acetone at 188 mM ¹H atom equivalents.
^{d}¹H-X5 (axial only) determined as the ratio of ¹H integration (4.08-4.12 ppm) to 1.00 for acetone at 188 mM ¹H atom equivalents.
^{e}The % MeGXₙ substrate converted to accumulated aldouronate products by each culture was determined on the basis of the X/MeG ratio found in the products generated from 0.5% MeGXₙ substrate after complete digestion with pure XynC. XynC digestion generated exclusively U-XOS containing a single MeG with a X/MeG ratio of 6.9 for integration of X1/U1 by ¹H-NMR. The concentration of MeGXₙ in the uninoculated medium was 5 mg ml⁻¹ and following the 3 x concentration of 3.0 ml of culture medium during the process of D₂O exchange prior to NMR analysis (Materials and Methods), the accumulated products would have been derived from 15 mg ml⁻¹ MeGXₙ. Using a MW for the product MeGX_{6.9} (191 + 5.9 × 132 + 150 = 1120 mg mmol⁻¹) the concentration of MeGX_{6.9} equivalents, equal to the concentration of MeG equivalents in the uninoculated medium, was 15 mg ml⁻¹/1120 mg mmol⁻¹ or 13.3 mM. This value, divided by the concentration of U1, provides an estimate of the fraction (%) of the MeGXₙ accumulated as products of MeGXₙ digestion.

### EXAMPLE 3

### Alternate Strategies for the Efficient Bioconversion of Methylglucoronoxylans

An efficient process for the depolymerization of MeGXₙ followed by the assimilation and metabolism of all of the products of depolymerization has been ascribed to bacteria that secrete GH10 endoxylanases and intracellularly process both the acidic U-XOS as well as the neutral XOS (12). Paenibacillus sp. JDR2 (Pjdr2) provides an example in which the efficient utilization of MeGXₙ involves extracellular depolymerization catalyzed by a cell-associated multimodular GH10 endoxylanase coupled with assimilation of aldouronates and XOS by ABC transporters and intracellular processing of U-XOS and XOS to xylose. The intracellular processing is catalyzed by a combination of glycoside hydrolases including a GH67 α-glucuronidase, a GH10 endoxylanase and a GH43 β-xylosidase/α-_{L}-arabinofuranosidse (14, 35). Based upon genomic sequences, these systems may occur in a few other bacteria as well.

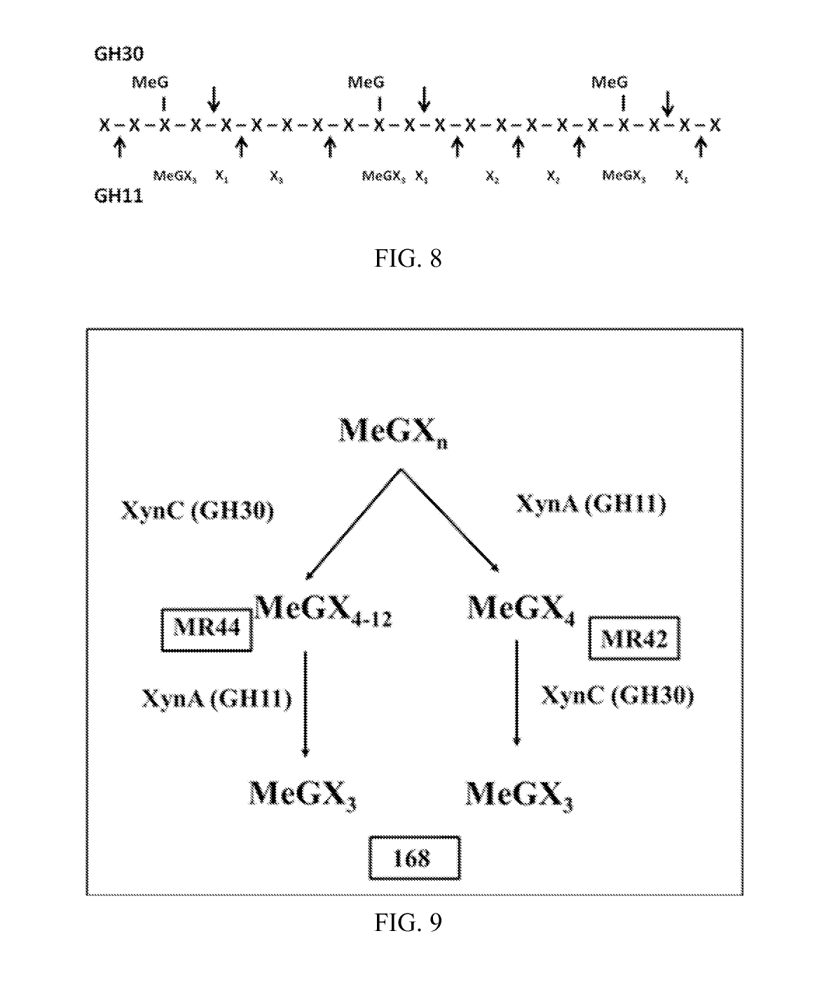

B. subtilis strain 168 has no gene encoding GH10 endoxylanases or GH67 α-glucuronidases and yet efficiently depolymerizes MeGXₙ and assimilates and metabolizes the neutral XOS X₂ and. X₃ generated by the combined action of the secreted. GH11 XynA and the GH30 XYNC enzymes. The combined action of these two xylanases on MeGXₙ is depicted below wherein the lower amount of xylose accumulates as MeGX₃ and the maximal amount as xylobiose and xylotriose which is generated for assimilation by ABC transporters. The scheme considers the combination of XynC and XynA acting on MeGXₙ with an average X to MeG ratio of 6.5 to 1 (see FIG. 8).

With a ratio of X to MeG of 6-7:1, an approximate average for MeGXₙ from sweetgum (Liquidambar syraciflua), the products of complete digestion would be X₁, X₂, X₃ and MeGX₃ with the neutral XOS representing approximately 50% of the total available for assimilation and metabolism. Both X₃ and X₂ would be rapidly assimilated by ABC transporters with slower assimilation of xylose. Bacteria that secrete a GH10 endoxylanase, generate X₂, X₃ and MeGX₃ in which the MeG is linked to the non-reducing terminal xylose, and produce a GH67 α-glucuronidase to process the assimilated MeGX₃ would allow greater yields of fermentation products from MeGXₙ with this level of MeG substitution. However, if the ratio of X to MeG reaches 20, as it may for the methylglucuronoarabinoxylans (MeGAXₙ) in the hemicellulose fraction of grasses, the GH30/GH11 xylanase combination may achieve utilization of 85% of the xylose without processing the MeGX₃. In this case B. subtilis and other bacteria that secrete GH11 and GH30 endoxylanases may be further developed as biocatalysts for the efficient fermentation of MeGAXₙ to targeted products.

U-XOS Accumulation by B. subtilis Strains with Deletions in xynA or xynC

The generation of a series of aldouronates with an increasing number of xylose residues and a single MeG linked α-1,2 to a xylose penultimate to the reducing terminal xylose is a characteristic of GH30 endoxylanases, with XynC from Bacillus subtilis (15, 16, 36) and XynA from Dickeya dadantii (previously Erwinia chrysanthemi) (25, 37, 38) as examples of these enzymes. For B. subtilis, the XynC generates few if any neutral XOS products for assimilation and metabolism from its action on the polymeric MeGXₙ. In contrast GH11 endoxylanases generate aldouronates in which MeG is linked α-1,2 to a xylose penultimate to the non-reducing terminal xylose with MeGX₄ as the limit product, along with xylotriose, xylobiose and some xylose (11).

The Examples disclosed herein confirm the products expected for the XynA and XynC from B. subtilis strain 168 with MeGXₙ from the hardwood, sweetgum. The path of carbon during growth on MeGX₁ may proceed sequentially through either XynA mediated depolymerization followed by XynC or first through XynC mediated depolymerization followed by XynA as in FIG. 9.

In the MR42 strain XynA is the only xylanase secreted, resulting in the expected limit for a GH11 endoxylanase of MeGX₄. As seen in the TLC analysis (FIG. 2) of the products generated by recombinant XynA, XynC, and the combination of XynA and XynC, XynA generates MeGX₄ but also significant levels of aldouronates with mobilities expected for MeGX₅ and MeGX₆. When XynC is present with XynA, MeGX₄ as well as the larger products are processed to MeGX₃.

MALDI-TOF MS provides profiles supporting the common identities of the products generated by XynC and the MR44 strain in which the gene encoding the GM. 1 XynA has been deleted, indicating that XynC is the only endoxylanase activity other than XynA that is secreted by B. subtilis strain 168. This is confirmed by the ¹H-NMR spectra of the XynC digest and the MR44 culture medium which structurally defines products and provides qualitative and quantitative information on the yields and average DP values of the accumulated aldouronates. The average DP values of accumulated products determined by the ratios of ¹H on C 1 or axial C5 on all xylose residues to the xylose on the reducing terminus are similar to the average xylose to methylglucuronate ratios (Table 6). This supports the process shown in FIG. 9 for the accumulation of aldouronates of different compositions by strains secreting only XynA, XynC or both enzymes. For the unmodified B. subtilis strain 168 the recovery of the MeG in the medium is 88% of the MeG provided in the substrate MeGXₙ, estimated from the ratio of X to MeG in the products accumulated in the MR44 strain. The estimated recovery of MeG in the MR44 strain is approximately the same at 86%. These estimated values are dependent on the accuracy of the integrations of different peaks from the ¹H-NMR spectra and may be subject to some error derived from the contributions to a given peak by more than a single ¹H atom. However the MeG recoveries for B. subtilis strain 168, MR44, as well as MR42 show essentially the same recovery of MeG, indicating that they can be used as biocatalysts for production of defined aldouronate mixtures.

Aldouronates, acidic xylooligosaccharides containing one or more methylglucuronate residues linked α-1,2 to xylose residues in the β-1,4-xylan backbone in methylglucuronoxylans, have been shown to have a range of immunomodulating and antimicrobial activities (4, 5, 39, 40). Acidic aldouronates (U-XOS) have received increasing attention for additional applications as well. Pentosan polysulfate (PPS) refers to products derived from U-XOS that are chemically sulfated to produce homologues of the naturally occurring glycosaminoglycan sulfates, heparin and chondroitin sulfate (5). PPS have been applied to the treatment of interstitial cystitis in humans (6) and osteoarthritis in horses (8, 41). Novel properties of PPS have been discovered that are expected to extend to treatment of disease associated with mucopolysaccharidoses (7).

The formation of PPS from pentosans involves the chemical sulfation of methylglucuronoxylans from hardwoods. A prominent source is wood from European beech which is subjected to thermochemical pretreatment to release the soluble MeGXₙ (42). Chemical sulfation provides a mixture of sulfated U-XOS that contain one or more uronic acids. The ability of B. subtilis to process glucuronoarabinoxylans and metabolize released arabinose, as well as metabolize α- and β-glucans, indicates the MR44 strain can be used to process impure preparations of hemicelluloses generated by the alkaline pretreatment of lignocellulosic biomass. The MR44 strain can serve as a biocatalyst to process hemicellulose fractions from various resources, including energy crops and agricultural residues, to provide pentosans for the production of PPS with defined composition for applications to human and veterinary medicine. The MR42 strain as well as B. subtilis strain 168 may also serve as biocatalysts for the production of MeGX₄ and MeGX₃ to develop applications for these acidic xylooligosaccharides.

### EXAMPLE 4

### Production of XOS by Strains of Bacillus Subtilis: Applications as Prebiotics and Probiotics

Xylooligosaccharides associated with promoting the growth of probiotic intestinal bacteria have been identified xylotiose and xylobiose which are prebiotics of value for applications in human and animal nutrition. These may be produced by a synthesis from monosaccharides or enzymatic digestion of xylans. Bacillus subtilis strains secrete a combination of GH11 and GH30 endoxylanases that collectively generate xylobiose and xylotriose as substrates for growth. Here we have inactivated genes within the genome of Bacillus subtilis required for the assimilation of xylobiose and xylotriose, resulting in the accumulation of these saccharides. The GRAS status of Bacillus subtilis strains supports the application of these strains for the production of these saccharides. These strains may serve as biocatalysts for the production of prebiotics or as probiotics for human and animal consumption.

Growth of B. subtilis Strains in which Insertion Inactivation of Genes Involved in Assimilation of Xylooligosaccharides.

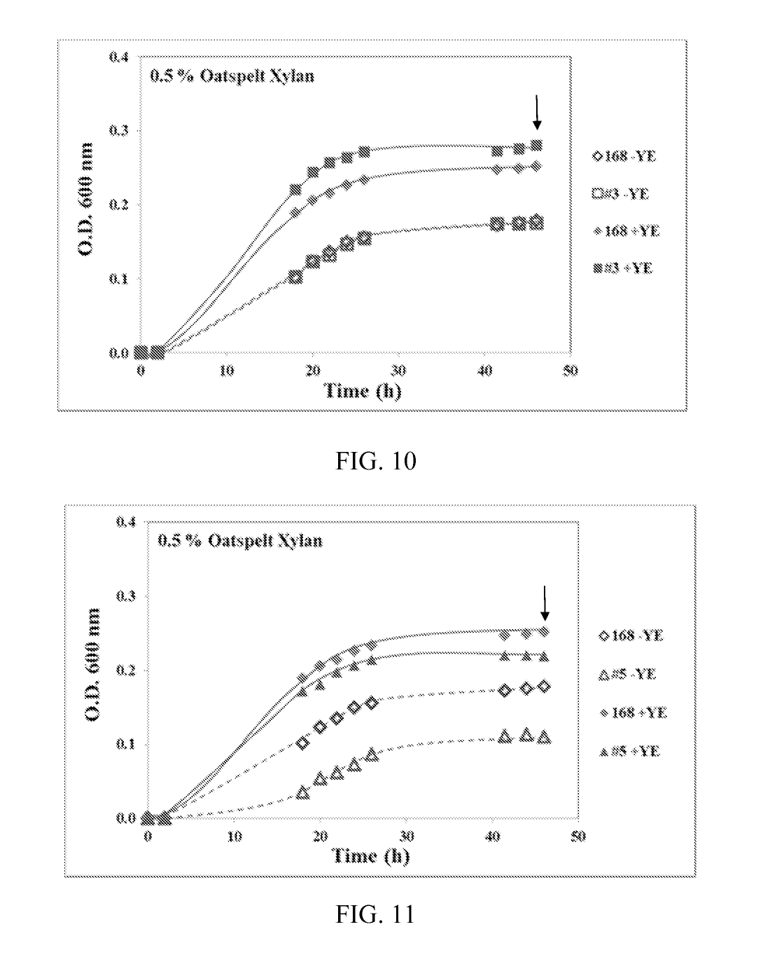

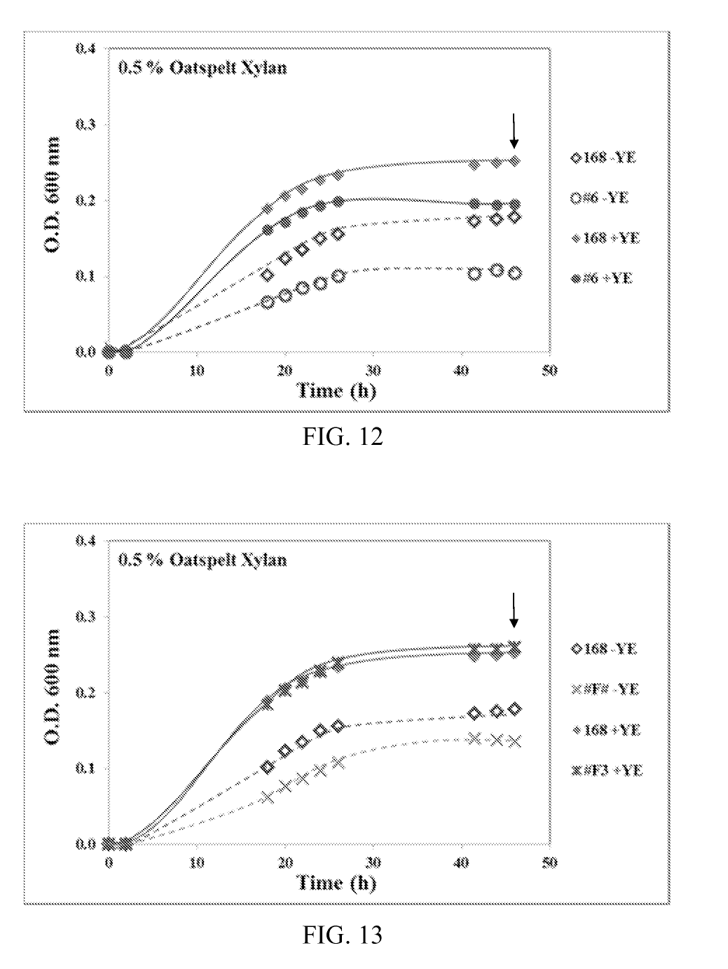

B. subtilis 168 cultures were treated with transposon Tn10, selected for growth on spectinomycin, followed by growth on Spizizen's medium containing xylooligosaccharides containing penicillin G. After 16 hours, cells were harvested, washed in LB, and suspended in LB for further cultivation. Cultures were inoculated into Spizizen medium containing cycloserine and xylologosaccharides. After 16 hours of culture, cells were spread on LB agar plates containing spectinomycin. Colonies were patched on to Spizizen agar plates containing XOS to identify and select mutants deficient in their ability to utilize XOS for growth with oat spelt xylan as substrate. Growth deficiencies representing accumulation of XOS is shown below for strain 3 (FIG. 10), 5 (FIG. 11), 6 (FIG. 12), F3 (FIG. 13).

### Accumulation of XOS Determined by TLC.

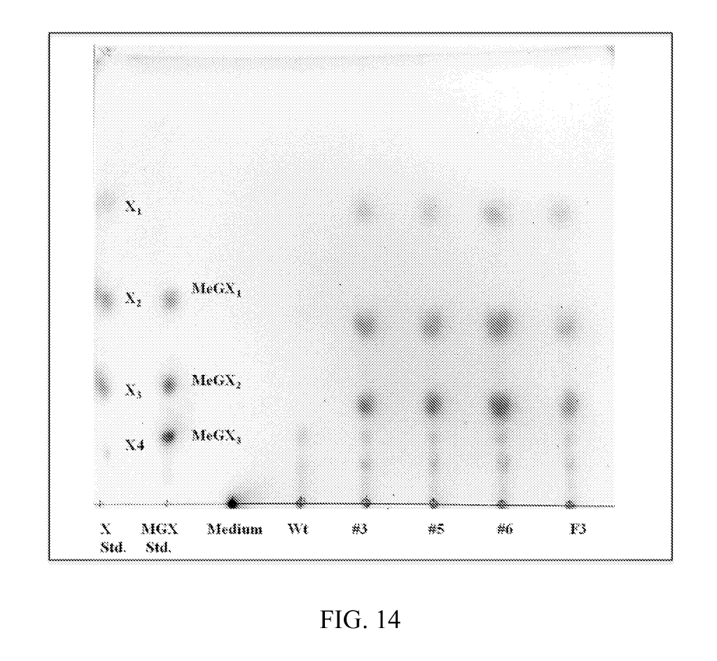

Samples taken from stationary phase cultures were analyzed by TLC as shown in FIG. 14. Saccharides detected with N-(1-Naphthyl) ethylenediamine dihydrochloride staining showed the accumulation of xylobiose and xylotriose along with small quantities of xylose. This demonstrates the abilities of all 4 stains to accumulate neutral oligosaccharides from oat spelt xylan as compared to medium and the non-mutagenized wild-type parent strain (B. subtilis 168).

Insertional inactivation sites were located in the yxxF gene encoding a putative transporter gene (SEQ ID NO: 7) for strain 3 (about 270 bp downstream from the start codon) and the kinC gene (SEQ ID NO: 10) encoding a regulatory gene associated with sporulation (SEQ ID NO: 9) was interrupted about 186 by downstream from start codon. As demonstrated above, inactivation of these genes results in accumulation of xylobiose and xylotriose.

### EXAMPLE 5

### Biological Activities of Sulfated Acidic Xylooligosaccharides Produced by Bacillus Subtilis Strain MR44

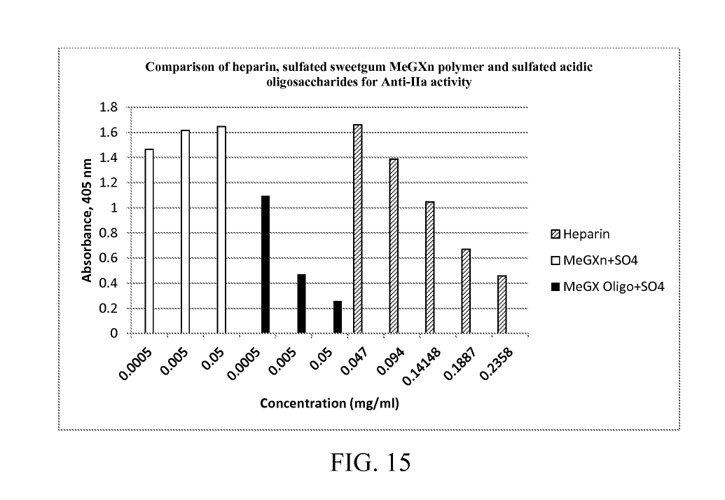

The inhibitory activities of the sulfated acidic oligosaccharides (UXOS) were compared with heparin for blocking the interaction of anti-thrombin with thrombin and thrombin activation for proteolytic release of chromogen from chromogenic peptide. Determinations were obtained based on the procedure for testing heparin inhibition of thrombin activity using a BIOPHEN HEPARIN ANTI-IIa kit (Hyphen BioMed) following conversion from international units of heparin to mg/ml starting concentrations for comparison with sulfated MeGXₙ or MeGX oligomer samples. Inactivation of thrombin activity over the ranges tested indicated 50% inhibition for heparin at 0.21 μg/ml while sulfated MeGX oligo showed a 50% inhibition at 5.6 μg/ml (see FIG. 15). Sulfated MeGXₙ polysaccharide showed no inhibition over the test range indicated below. On a weight basis heparin was 26.7 times more effective than sulfated oligosaccharides at inhibiting thrombin activation.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

TABLE 7

| organism | Family 10 | Family 11 | Family 30 | Family 43 | Family 8 | Family 39 |
|---|---|---|---|---|---|---|
| [Caldibacillus] cellulovorans | Q9L8L8 | | | | | |
| Acanthamoeba castellanii str. | L8GNI7 | | | | | |
| Neff | | | | | | |
| Acetobacteraceae bacterium | H0A1L5; | | | | | |
| AT-5844 | H0A6N5 | | | | | |
| Acidiphilium cryptum (strain JF- | | | | | | A5FT29 |
| 5) | | | | | | |
| Acidithiobacillus ferrivorans | | | | | G0JQE3; | |
| SS3 | | | | | G0JSC5; | |
| | | | | | G0JST7 | |
| Acidobacterium capsulatum | Q9AJR9 | | | | | |
| Acidobacterium capsulatum | C1F8L6 | | | | | |
| (strain ATCC 51196/DSM | | | | | | |
| 11244/JCM 7670) | | | | | | |
| Acidobacterium sp. (strain | E8X6C6 | | | | | |
| MP5ACTX9) | | | | | | |
| Acidobacterium sp. (strain | | | | E8WZI9; | | E8WV76; |
| MP5ACTX9) | | | | E8X5Y9; | | E8X2S4; |
| | | | | E8X699 | | E8X479 |
| Acidomyces acidophilus | | Q6VAY1 | | | | |
| Acidothermus cellulolyticus | A0LR95; | | | | | |
| (strain ATCC 43068/11B) | A0LRT6 | | | | | |
| Acidovorax citrulli (strain | A1TT53 | | | | | |
| AAC00-1) (Acidovorax avenae | | | | | | |
| subsp. citrulli) | | | | | | |
| Acinetobacter soli NIPH 2899 | N9BZN5 | | | | | |
| Acrophialophora nainiana | | Q0ZBK9; | | | | |
| | | Q0ZBL0 | | | | |
| Actinomadura sp. | Q59139 | | | | | |
| Actinomadura sp. S14 | | F1SX84 | | | | |
| Actinoplanes missouriensis | I0H4S4; | I0H6D1 | | I0H4T2; | | |
| (strain ATCC 14538/DSM | I0H4W2; | | | I0HDA6 | | |
| 43046/CBS 188.64/JCM 3121/ | I0H633; | | | | | |
| NCIMB 12654/NBRC 102363/ | I0H995; | | | | | |
| 431) | I0HFW5 | | | | | |
| Actinoplanes sp. (strain ATCC | G8S118; | G8SKM4 | | G8S0V2; | | |
| 31044/CBS 674.73/SE50/110) | G8S9Z5; | | | G8S3K4 | | |
| | G8SA02; | | | | | |
| | G8SA06; | | | | | |
| | G8SB12; | | | | | |
| | G8SM07 | | | | | |
| Actinoplanes sp. N902-109 | | | | R4LM91 | | |
| Actinosynnema mirum (strain | C6W8M3; | C6WIK2 | | C6W8M2; | | |
| ATCC 29888/DSM 43827/ | C6WMJ4; | | | C6W8V9; | | |
| NBRC 14064/IMRU 3971) | C6WN49; | | | C6WAZ0; | | |
| | C6WS74 | | | C6WD14; | | |
| | | | | C6WQB2 | | |
| Actinosynnema pretiosum | F5APW6 | | | F5APW5 | | |
| subsp. auranticum | | | | | | |
| Aegilops tauschii (Tausch's | M8BJB3; | | | | | |
| goatgrass) (Aegilops squarrosa) | M8BXH8; | | | | | |
| | M8C4H2; | | | | | |
| | M8CG38; | | | | | |
| | M8CHS6; | | | | | |
| | M8CPA9; | | | | | |
| | M8CZM5; | | | | | |
| | N1QVN9 | | | | | |
| Aeromonas punctata | O83007; | Q43993 | | | | |
| (Aeromonas caviae) | Q9485 | | | | | |
| Afipia felis ATCC 53690 | K8NQ21 | | | | | |
| Afipia sp. 1NLS2 | D6V6G1 | | | | | |
| Agaricus bisporus (White | O60206; | | | | | |
| button mushroom) | Q9HGX1 | | | | | |
| Agaricus bisporus var. bisporus | K9I812; | K9H866 | | | | |
| (strain H97/ATCC MYA-4626/ | K9ICQ8 | | | | | |
| FGSC 10389) (White button | | | | | | |
| mushroom) | | | | | | |
| Agaricus bisporus var. burnettii | K5VIU0; | K5VRL4 | | K5XVY4 | | |
| (strain JB137-S8/ATCC MYA- | K5X6J7 | | | | | |
| 4627/FGSC 10392) (White | | | | | | |
| button mushroom) | | | | | | |
| Agrobacterium sp. (strain H13- | F0L9D5 | | | | | |
| 3) (Rhizobium lupini (strain | | | | | | |
| H13-3)) | | | | | | |
| Agrobacterium sp. ATCC 31749 | F5J970 | | | | | |
| Agrobacterium tumefaciens | Q7CX80 | | | | | |
| (strain C58/ATCC 33970) | | | | | | |
| Agrobacterium tumefaciens 5A | H0H873 | | | | | |
| Agrobacterium tumefaciens | G6Y0L9 | | | | | |
| CCNWGS0286 | | | | | | |
| Agrobacterium tumefaciens F2 | F7U5F0 | | | | | |
| Agrobacterium tumefaciens str. | M8B6Y5 | | | | | |
| Cherry 2E-2-2 | | | | | | |
| Ajellomyces capsulata (strain | | | | C0P159 | | |
| G186AR/H82/ATCC MYA- | | | | | | |
| 2454/RMSCC 2432) (Darling's | | | | | | |
| disease fungus) (Histoplasma | | | | | | |
| capsulatum) | | | | | | |
| Ajellomyces capsulata (strain | | | | C6HSE3 | | |
| H143) (Darling's disease | | | | | | |
| fungus) (Histoplasma | | | | | | |
| capsulatum) | | | | | | |
| Ajellomyces capsulata (strain | | | | F0UVZ7 | | |
| H88) (Darling's disease fungus) | | | | | | |
| (Histoplasma capsulatum) | | | | | | |
| Ajellomyces capsulata (strain | | | | A6R3B9 | | |
| NAm1/WU24) (Darling's | | | | | | |
| disease fungus) (Histoplasma | | | | | | |
| capsulatum) | | | | | | |
| Ajellomyces dermatitidis (strain | | | | F2T2L9 | | |
| ATCC 18188/CBS 674.68) | | | | | | |
| (Blastomyces dermatitidis) | | | | | | |
| Ajellomyces dermatitidis (strain | | | | C5GG36 | | |
| ER-3/ATCC MYA-2586) | | | | | | |
| (Blastomyces dermatitidis) | | | | | | |
| Ajellomyces dermatitidis (strain | | | | C5JGV8 | | |
| SLH14081) (Blastomyces | | | | | | |
| dermatitidis) | | | | | | |
| Algoriphagus sp. PR1 | A3HZ47 | | | A3HSX1; | | |
| | | | | A3HV06 | | |
| Alicyclobacillus acidocaldarius | F8II24 | | | | | |
| (strain Tc-4-1) (Bacillus | | | | | | |
| acidocaldarius) | | | | | | |
| Alicyclobacillus acidocaldarius | B7DSC7 | | | | | |
| LAA1 | | | | | | |
| Alicyclobacillus acidocaldarius | C8WRS4 | | | | | |
| subsp. acidocaldarius (strain | | | | | | |
| ATCC 27009/DSM 446/104- | | | | | | |
| 1A) (Bacillus acidocaldarius) | | | | | | |
| Alicyclobacillus hesperidum | J9HN03; | | | | | |
| URH17-3-68 | J9HNF2 | | | | | |
| Alicyclobacillus sp. A15 | D8UVH0 | | | | | |
| Alicyclobacillus sp. A4 | D3VWB5; | | | | | |
| | E2EAK3 | | | | | |
| Alistipes sp. CAG:268 | | | | R6W417 | | |
| Alternaria alternata (Alternaria | Q9UVP5 | | | | | |
| rot fungus) (Torula alternata) | | | | | | |
| Alternaria sp. HB186 | | Q0Q592 | | | | |
| Alteromonas macleodii (strain | K0D9S6 | | K0D9S1 | | | |
| Black Sea 11) | | | | | | |
| Alteromonas macleodii AltDE1 | K7RHS4 | | | K7RFZ1; | | |
| | | | | K7RYS4 | | |
| Alteromonas macleodii ATCC | J9YEG0 | | | J9YEF7 | | |
| 27126 | | | | | | |
| Amphibacillus xylanus (strain | K0IZE4; | K0J5P9 | | | | |
| ATCC 51415/DSM 6626/JCM | K0IZL9; | | | | | |
| 7361/LMG 17667/NBRC | K0J7S2 | | | | | |
| 15112/Ep01) | | | | | | |
| Ampullaria crossean | B2Z4D8; | | | | | |
| | Q29U71; | | | | | |
| | Q7Z1V6 | | | | | |
| Amycolatopsis mediterranei | D8HPV0; | D8I8A6 | | D8HTR6; | | |
| (strain U-32) | D8HPW0; | | | D8I8X3 | | |
| | D8HTZ0; | | | | | |
| | D8HYL7; | | | | | |
| | D8I044; | | | | | |
| | D8I5I7; | | | | | |
| | D8I9B1 | | | | | |
| Amycolatopsis mediterranei | G0FIL2; | G0G8N8 | | G0FM13; | | |
| S699 | G0FJ20; | | | G0FXW4 | | |
| | G0FJ30; | | | | | |
| | G0FJD9; | | | | | |
| | G0FJL4; | | | | | |
| | G0FNA1; | | | | | |
| | G0FTR6; | | | | | |
| | I7CYW5; | | | | | |
| | I7DNX6 | | | | | |
| Amycolatopsis orientalis | | | | R4T6F6 | | |
| HCCB10007 | | | | | | |
| Amycolatopsis vancoresmycina | R1G1I7; | R1I386 | | R1FMQ1 | | |
| DSM 44592 | R1GF48; | | | | | |
| | R1HML0; | | | | | |
| | R1I729 | | | | | |
| Anabaena cylindrica (strain | | | | K9ZGC2 | | |
| ATCC 27899/PCC 7122) | | | | | | |
| Anabaena variabilis (strain | Q3MDD9 | | | | | |
| ATCC 29413/PCC 7937) | | | | | | |
| Anaerotruncus sp. CAG:528 | | | | R5XB21; | | |
| | | | | R5XCH8 | | |
| Annulohypoxylon stygium | | B0FX61 | | | | |
| Anoxybacillus sp. E2(2009) | D7NNK8 | | | | | |
| Arabidopsis lyrata subsp. lyrata | D7KKL5; | | | | | |
| (Lyre-leaved rock-cress) | D7KY62; | | | | | |
| | D7MFM0 | | | | | |
| | G1JSH1; | | | | | |
| | O80596; | | | | | |
| | Q84VX1; | | | | | |
| | Q9C643; | | | | | |
| | Q9SM08; | | | | | |
| | Q9SVF5; | | | | | |
| | Q9SYE3; | | | | | |
| | Q9SZP3 | | | | | |
| Arcticibacter svalbardensis | | | R9GRT2; | R9GU46; | | |
| MN12-7 | | | R9GSB2; | R9GXJ1 | | |
| | | | R9H2T6 | | | |
| Arthrobacter chlorophenolicus | B8HDP0 | | | B8H6G1; | | |
| (strain A6/ATCC 700700/DSM | | | | B8HAG8; | | |
| 12829/JCM 12360) | | | | B8HAN9; | | |
| | | | | B8HHI2 | | |
| Arthrobacter | F0M694; | | | | | |
| phenanthrenivorans (strain | F0M699; | | | | | |
| DSM 18606/JCM 16027/LMG | F0M6Y7 | | | | | |
| 23796/Sphe3) | | | | | | |
| Arthrobacter sp. (strain FB24) | | | | A0JRF8 | | |
| Arthrobacter sp. SJCon | | | | L8TQS8; | | |
| | | | | L8TRF1 | | |
| Arthrobotrys oligospora (strain | G1XA53; | G1X0J7 | | | | |
| ATCC 24927/CBS 115.81/ | G1XDN2; | | | | | |
| DSM 1491) (Nematode- | G1XGJ3; | | | | | |
| trapping fungus) | G1XM94 | | | | | |
| (Didymozoophaga oligospora) | | | | | | |
| Ascochyta rabiei | | Q9UW04 | | | | |
| Aspergillus aculeatus | O59859 | F2ZAD6 | | Q9HFS9 | | |
| Aspergillus awamori (Black koji | | C6F1J6; | | | | |
| mold) | | P55328 | | | | |
| Aspergillus cf. niger BCC14405 | | Q6QA21 | | | | |
| Aspergillus clavatus (strain | A1CHQ0; | A1CCU0; | | A1CK29; | | |
| ATCC 1007/CBS 513.65/DSM | A1CUK2 | A1CD49; | | A1CLG4; | | |
| 816/NCTC 3887/NRRL 1) | | A1CU59 | | A1CN18; | | |
| | | | | A1CN93 | | |
| Aspergillus flavus (strain ATCC | B8NER4; | B8NGW8; | | B8MZR9; | | |
| 200026/FGSC A1120/NRRL | B8NIB9; | B8NJ86; | | B8N803; | | |
| 3357/JCM 12722/SRRC 167) | B8NXJ2; | B8NKE9; | | B8NDL1; | | |
| | B8NXT6 | B8NYB7 | | B8NMD3 | | |
| Aspergillus japonicus | | D3KT79 | | | | |
| Aspergillus kawachii (strain | P33559 | G7XIG9; | | G7XCF3; | | |
| NBRC 4308) (White koji mold) | | G7XTX6; | | G7XDP0; | | |
| (Aspergillus awamori var. | | P33557; | | G7XI38; | | |
| kawachi) | | P48824 | | G7XTG2; | | |
| Aspergillus niger | C5J411 | B0LUX1; | | P42256 | | |
| | | C0LZ11; | | | | |
| | | E3UN71; | | | | |
| | | F5CI28; | | | | |
| | | I3QKR8; | | | | |
| | | I3QKR9; | | | | |
| | | P55329; | | | | |
| | | P55330; | | | | |
| | | Q12549; | | | | |
| | | Q12550; | | | | |
| | | Q45F01; | | | | |
| | | Q6QJ75; | | | | |
| | | Q9C1G6; | | | | |
| | | Q9HGU0 | | | | |
| Aspergillus niger (strain ATCC | G3Y866 | G3XSA3; | | G3XM71; | | |
| 1015/CBS 113.46/FGSC | | G3XTQ6; | | G3Y1C5; | | |
| A1144/LSHB Ac4/NCTC | | G3XY88 | | G3Y1I3; | | |
| 3858a/NRRL 328/USDA | | | | G3Y5N7 | | |
| 3528.7) | | | | | | |
| Aspergillus niger (strain CBS | A2QFV7 | A2Q7I0; | | A2QT85; | | |
| 513.88/FGSC A1513) | | A2QBA9; | | A2R794; | | |
| | | A2R4D1; | | A5AAG2 | | |
| | | A2R5J7 | | | | |
| Aspergillus niveus | | G9FXH4 | | H6TQN0 | | |
| Aspergillus oryzae (strain | I7ZZI5; | I7ZZ52; | | I7ZVJ1; | | |
| 3.042) (Yellow koji mold) | I8I8T7; | I8A4X2; | | I8A6C0; | | |
| | I8IFG1; | I8TIC5; | | I8IBF4; | | |
| | I8IUT2; | I8TSN5 | | I8TQC6; | | |
| | I8TIW6 | | | I8U2R0 | | |
| Aspergillus oryzae (strain ATCC | O94163; | P87037; | | Q2U1X8; | | |
| 42149/RIB 40) (Yellow koji | Q2TYA7; | Q2TYR4; | | Q2U8C6; | | |
| mold) | Q2U7D0; | Q2UFR7; | | Q2UI74; | | |
| | Q96VB6 | Q9HFA4 | | Q2UQB3 | | |
| Aspergillus oryzae (Yellow koji | J7FK35 | H6WWN7 | | | | |
| mold) | | | | | | |
| Aspergillus saitoi (Aspergillus | | Q2PQU3 | | | | |
| phoenicis) | | | | | | |
| Aspergillus sojae | Q9P955 | | | | | |
| Aspergillus sulphureus | | Q2I0I8; | | | | |
| | | Q3S401 | | | | |
| Aspergillus terreus | H9BYX9; | | | | | |
| | Q4JHP5 | | | | | |
| Aspergillus terreus (strain NIH | Q0CBM8; | Q0CFS3; | | Q0CRJ6; | | |
| 2624/FGSC A1156) | Q0CGK2; | Q0CMZ1 | | Q0CS14; | | |
| | Q0CSC4; | | | Q0CXM2; | | |
| | Q0CZS5 | | | Q0CY27 | | |
| Aspergillus tubingensis | | P55331 | | | | |
| Aspergillus usamii | E9NSU0 | A6N2L7; | | | | |
| | | A6N2L8; | | | | |
| | | A6N2L9; | | | | |
| | | A6N2M0; | | | | |
| | | A6N2M1; | | | | |
| | | A6N2M2; | | | | |
| | | G0YP25; | | | | |
| | | G0YP27; | | | | |
| | | G0YP28; | | | | |
| | | Q2PU02; | | | | |
| | | Q45UD8 | | | | |
| Aspergillus versicolor | A2I7V1 | A2I7V2 | | | | |
| Asticcacaulis biprosthecum C19 | F4QMI5; | | | F4QI21 | | |
| | F4QR47; | | | | | |
| | F4QTP8 | | | | | |
| Asticcacaulis excentricus (strain | E8RR99; | | | E8RMF7; | | E8RKQ9 |
| ATCC 15261/DSM 4724/VKM | E8RRD7; | | | E8RN95; | | |
| B-1370/CB 48) | E8RTY3 | | | E8RNQ4; | | |
| | | | | E8RPF6; | | |
| | | | | E8RRD6; | | |
| | | | | E8RS53; | | |
| | | | | E8RS54; | | |
| | | | | E8RTM2; | | |
| | | | | E8RV37; | | |
| | | | | E8RVB9 | | |
| Aureobasidium pullulans (Black | | Q12562; | | | | |
| yeast) (Pullularia pullulans) | | Q9UW17 | | | | |
| Aureobasidium pullulans var. | Q2PGV8 | Q96TR7 | | | | |
| melanogenum | | | | | | |
| Auricularia delicata (strain | J0CXB2 | | | J0LGH4 | | |
| TFB10046) (White-rot fungus) | | | | | | |
| Azospirillum brasilense Sp245 | G8ATD6; | | | | | |
| | G8AWL3 | | | | | |
| Azospirillum lipoferum (strain | G7ZBN5 | | | | | |
| 4B) | | | | | | |
| Azospirillum sp. (strain B510) | D3P0M1 | | | | | |
| Bacillus agaradhaerens (Bacillus | | Q7SIE2; | | | | |
| agaradherans) | | Q7SIE3 | | | | |
| Bacillus alcalophilus | Q6TDT4 | | | | | |
| Bacillus amyloliquefaciens | | B5M6I0; | | F4EIU7 | | |
| (Bacillus velezensis) | | E0YL13; | | | | |
| | | F4EK86; | | | | |
| | | Q45VU6 | | | | |
| Bacillus amyloliquefaciens | | E1UUS4 | | E1UMM6 | | |
| (strain ATCC 23350/DSM 7/ | | | | | | |
| BCRC 11601/NBRC 15535/ | | | | | | |
| NRRL B-14393) | | | | | | |
| Bacillus amyloliquefaciens | | A7Z9N2 | | A7Z7G9 | | |
| (strain FZB42) | | | | | | |
| Bacillus amyloliquefaciens IT-45 | | M1KM92 | | M1JXX2 | | |
| Bacillus amyloliquefaciens | | L0BRQ4 | | L0BRH7 | | |
| subsp. plantarum AS43.3 | | | | | | |
| Bacillus amyloliquefaciens | | H2AFD2 | | H2AAU4 | | |
| subsp. plantarum CAU B946 | | | | | | |
| Bacillus amyloliquefaciens | | K2I2H3 | | K2IGC9 | | |
| subsp. plantarum M27 | | | | | | |
| Bacillus amyloliquefaciens | | M1XF26 | | M1XEG7 | | |
| subsp. plantarum UCMB5036 | | | | | | |
| Bacillus amyloliquefaciens | | H8XJ69 | | H8XEP9 | | |
| subsp. plantarum YAU B9601- | | | | | | |
| Y2 | | | | | | |
| Bacillus amyloliquefaciens | | F4E5M4; | | F4E9U5 | | |
| TA208 | | F4E5N1 | | | | |
| Bacillus amyloliquefaciens XH7 | | | | G0ILJ8 | | |
| Bacillus amyloliquefaciens XH7 | | G0INC1 | | | | |
| Bacillus amyloliquefaciens Y2 | | | | I2C8R7 | | |
| Bacillus atrophaeus (strain | | | | E3E0X7 | | |
| 1942) | | | | | | |
| Bacillus atrophaeus C89 | | | | I4XM48 | | |
| Bacillus atrophaeus UCMB- | | | | R0MM55 | | |
| 5137 | | | | | | |
| Bacillus cellulosilyticus (strain | E6TXK9; | | E6U0Q3 | E6TQD4; | E6TXK5 | |
| ATCC 21833/DSM 2522/ | E6TXL5 | | | E6TQD7 | | |
| FERM P-1141/JCM 9156/N-4) | | | | | | |
| Bacillus cereus | | Q45VU3 | | | | |
| Bacillus circulans | | P09850; | | | P19254 | |
| | | Q8RMN8 | | | | |
| Bacillus coagulans 36D1 | G2TR15 | | | | | |
| Bacillus firmus | Q6U892 | Q6U894; | | | | |
| | | Q71S35 | | | | |
| Bacillus halodurans | M4QNR9; | Q79MJ7 | | | | |
| | Q17TM8; | | | | | |
| | Q546Y2 | | | | | |
| Bacillus halodurans (strain | P07528 | Q9KEF3 | | | Q9KB30 | |
| ATCC BAA-125/DSM 18197/ | | | | | | |
| FERM 7344/JCM 9153/C-125) | | | | | | |
| Bacillus licheniformis | | A5H0S3; | | D0FZZ4; | | |
| | | B5SYI8; | | H1AD40; | | |
| | | Q45VU7 | | H1AD41 | | |
| Bacillus licheniformis (strain | | | | Q65D31; | | |
| DSM 13/ATCC 14580) | | | | Q65GB9; | | |
| | | | | Q65L63; | | |
| | | | | Q65MB6; | | |
| | | | | Q65MB7 | | |
| Bacillus licheniformis WX-02 | | | | I0UEC7; | | |
| | | | | I0UJ59 | | |
| Bacillus megaterium | | C7DZC1 | | | | |
| Bacillus pumilus (Bacillus | Q8L2X3 | B1A4I1; | | P07129 | | |
| mesentericus) | | C8CB65; | | | | |
| | | E2IHA1; | | | | |
| | | I3RYY0; | | | | |
| | | I7B1S7; | | | | |
| | | J7F591; | | | | |
| | | P00694; | | | | |
| | | Q06RH9; | | | | |
| | | Q45VU4; | | | | |
| | | Q5EFR9; | | | | |
| | | Q8RMN7; | | | | |
| | | Q9AMB5; | | | | |
| | | Q9L7Q9 | | | | |
| Bacillus pumilus (strain SAFR- | A8FDC5 | A8FE31 | | | | |
| 032) | | | | | | |
| Bacillus pumilus ATCC 7061 | B4ADW4 | B4AL14 | | | | |
| Bacillus selenitireducens (strain | | | | D6XWN2 | | |
| ATCC 700615/DSM 15326/ | | | | | | |
| MLS10) | | | | | | |
| Bacillus sonorensis L12 | | M5P205 | | M5P739 | | |
| Bacillus sp. | Q45518 | Q9ZB36 | | | | |
| Bacillus sp. (strain KSM-330) | | | | | P29019 | |
| Bacillus sp. 31 | G4XVR8 | G4XVR9 | | | | |
| Bacillus sp. 41M-1 | | Q9RC94 | | | | |
| Bacillus sp. 5B6 | | I2HW91 | | I2HTZ5 | | |
| Bacillus sp. 916 | | J0XBX4 | | J0DF79 | | |
| Bacillus sp. BP-7 | | Q84F19 | | | | |
| Bacillus sp. BT1B_CT2 | | | | E5W4P3; | | |
| | | | | E5W6I9 | | |
| Bacillus sp. HBP8 | | Q58G72 | | | | |
| Bacillus sp. HJ2 | I6PB27 | | | | | |
| Bacillus sp. JB 99 | G1E731 | | | | | |
| Bacillus sp. JB99 | | D2KPJ0 | | | | |
| Bacillus sp. JS | | I0F4P8; | | I0F7F1 | | |
| | | I0F545 | | | | |
| Bacillus sp. M 2-6 | I4VBY2 | I4VA71 | | | | |
| Bacillus sp. N16-5 | D7RA44 | | | | | |
| Bacillus sp. NBL420 | | Q8VVC3 | | | | |
| Bacillus sp. NCL 87-6-10 | | G4XQJ9; | | | | |
| | | G4XQK0; | | | | |
| | | G4XQK1 | | | | |
| Bacillus sp. NG-27 | O30700 | | | | | |
| Bacillus sp. SN5 | L0CL88 | | | | | |
| Bacillus sp. YA-14 | | Q59256 | | | | |
| Bacillus sp. YA-335 | | Q59257 | | | | |
| Bacillus sp. YJ6 | | C5MTD6 | | | | |
| Bacillus stratosphericus LAMA | M5RDI6 | M5QXB9 | | | | |
| 585 | | | | | | |
| Bacillus subtilis | | B9ZZN9; | Q6YK37 | D6RV88; | | |
| | | C6F1T5; | | O07078 | | |
| | | C7F433; | | | | |
| | | D7F2D8; | | | | |
| | | E0YTQ6; | | | | |
| | | F6LP55; | | | | |
| | | F6LP56; | | | | |
| | | K7QVW4; | | | | |
| | | M4YBE9; | | | | |
| | | Q3HLJ4; | | | | |
| | | Q45VU1; | | | | |
| | | Q45VU2; | | | | |
| | | Q59254; | | | | |
| | | Q7SID8; | | | | |
| | | Q8RMN9 | | | | |
| Bacillus subtilis (strain 168) | | P18429 | Q45070 | P42293; | | |
| | | | | P94489; | | |
| | | | | P94522; | | |
| | | | | Q45071 | | |
| Bacillus subtilis (strain BSn5) | | E8VJZ4 | | E8VGJ7 | | |
| Bacillus subtilis BEST7003 | | | | N0DIN5 | | |
| Bacillus subtilis BEST7003 | | N0DC67 | | | | |
| Bacillus subtilis MB73/2 | | M2W0R4 | | M2VKA1 | | |
| Bacillus subtilis QB928 | | J7JNY2 | | J7JQH8 | | |
| Bacillus subtilis subsp. | | L8PW24; | | L8PXI0 | | |
| inaquosorum KCTC 13429 | | L8Q1B0 | | | | |
| Bacillus subtilis subsp. spizizenii | | E0TVS7 | | E0TW09 | | |
| (strain ATCC 23059/NRRL B- | | | | | | |
| 14472/W23) | | | | | | |
| Bacillus subtilis subsp. spizizenii | | D5MZA5 | | D5N0Z4 | | |
| ATCC 6633 | | | | | | |
| Bacillus subtilis subsp. spizizenii | | G4NPX3; | | G4NYV7 | | |
| TU-B-10 | | G4NVR8 | | | | |
| Bacillus subtilis subsp. subtilis | | M1UM93 | | M1TCZ1 | | |
| 6051-HGW | | | | | | |
| Bacillus subtilis subsp. subtilis | | M4X9P9 | | M4XFY9 | | |
| str. BAB-1 | | | | | | |
| Bacillus subtilis subsp. subtilis | | | | L0D2X9 | | |
| str. BSP1 | | | | | | |
| Bacillus subtilis subsp. subtilis | | L0D1U8 | | | | |
| str. BSP1 | | | | | | |
| Bacillus subtilis subsp. subtilis | | G4PAY1 | | G4PBF1 | | |
| str. RO-NN-1 | | | | | | |
| Bacillus subtilis subsp. subtilis | | G4EVI3 | | G4ESN0 | | |
| str. SC-8 | | | | | | |
| Bacillus subtilis XF-1 | | | | M4KY09 | | |
| Bacillus thermodenitrificans | B2Z4E4; | | | Q93HT9 | | |
| | G5CKS2 | | | | | |
| Bacterium enrichment culture | K0H4D9 | | | | | |
| clone MC3F | | | | | | |
| Bacterium enrichment culture | | H9ZGD1 | | | | |
| clone Xyl8B8 | | | | | | |
| Bacteroides cellulosilyticus | I8VGX9; | | | I8W4H9; | | |
| CL02T12C19 | I8VZ35; | | | I9QTS4 | | |
| | I9R905 | | | | | |
| Bacteroides cellulosilyticus | E2NA18; | | | E2NBW2; | | |
| DSM 14838 | E2NE69; | | | E2NBW3; | | |
| | E2NGI7; | | | E2NCH0 | | |
| | E2NGL0 | | | | | |
| Bacteroides coprocola DSM | | | | B3JNI4 | | |
| 17136 | | | | | | |
| Bacteroides dorei 5_1_36/D4 | C3R891 | | | C3RFH5 | | |
| Bacteroides dorei CAG:222 | | | | R6HWA3; | | |
| | | | | R6HWC0; | | |
| | | | | R6I0U1; | | |
| | | | | R6IFQ3 | | |
| Bacteroides dorei CL02T00C15 | I8VPP7 | | | I8VXM5 | | |
| Bacteroides dorei CL02T12C06 | I9QWT8 | | | I9FX45 | | |
| Bacteroides dorei CL03T12C01 | I9FXV7 | | | I8WNJ9 | | |
| Bacteroides dorei DSM 17855 | B6VTT4 | | | | | |
| Bacteroides eggerthii | E5WUX6; | | | | | |
| 1_2_48FAA | E5WZP4; | | | | | |
| | E5WZR2 | | | | | |
| Bacteroides eggerthii DSM | B7AFX4; | | | | | |
| 20697 | B7AFZ5; | | | | | |
| | B7AIY1 | | | | | |
| Bacteroides finegoldii | K5BVR1; | | | K5CP77 | | |
| CL09T03C10 | K5C8F1; | | | | | |
| | K5C8G7 | | | | | |
| Bacteroides finegoldii DSM | | | | C9KR69 | | |
| 17565 | | | | | | |
| Bacteroides fragilis (strain | | | | E1WMA7 | | |
| 638R) | | | | | | |
| Bacteroides fragilis (strain ATCC | | | | Q5LIF9 | | |
| 25285/NCTC 9343) | | | | | | |
| Bacteroides fragilis (strain | | | | Q64ZI3 | | |
| YCH46) | | | | | | |
| Bacteroides fragilis 3_1_12 | | | | E4VW62 | | |
| Bacteroides fragilis CAG:558 | | | | R5RW99 | | |
| Bacteroides fragilis | | | | I9SEJ2 | | |
| CL03T00C08 | | | | | | |
| Bacteroides fragilis | | | | I9S668 | | |
| CL03T12C07 | | | | | | |
| Bacteroides fragilis | | | | I9B2C7 | | |
| CL05T00C42 | | | | | | |
| Bacteroides fragilis | | | | I9W043 | | |
| CL05T12C13 | | | | | | |
| Bacteroides fragilis | | | | I3HT65 | | |
| CL07T00C01 | | | | | | |
| Bacteroides fragilis | | | | I9KJL1 | | |
| CL07T12C05 | | | | | | |
| Bacteroides fragilis HMW 610 | | | | K1G3N2 | | |
| Bacteroides fragilis HMW 615 | | | | K1FXT9 | | |
| Bacteroides fragilis HMW 616 | | | | K1FD41 | | |
| Bacteroides helcogenes (strain | | | | E6SMV7; | | |
| ATCC 35417/DSM 20613/ | | | | E6SMV8 | | |
| JCM 6297/P 36-108) | | | | | | |
| Bacteroides intestinalis | | | | R7DX30 | | |
| CAG:315 | | | | | | |
| Bacteroides intestinalis DSM | B3C594; | | | B3C9W4; | | |
| 17393 | B3C6N3; | | | B3C9W5 | | |
| | B3CER6; | | | | | |
| | B3CES1 | | | | | |
| Bacteroides nordii CL02T12C05 | I9SCM6 | | | | | |
| Bacteroides oleiciplenus YIT | K9DZN1; | | | K9E179; | | |
| 12058 | K9E2G7; | | | K9E2P1; | | |
| | K9E2I1; | | | K9EP01 | | |
| | K9E3N2; | | | | | |
| | K9EGG3 | | | | | |
| Bacteroides ovatus | B2KZK5; | | | P49943 | | |
| | P49942 | | | | | |
| Bacteroides ovatus 3_8_47FAA | F7LA32; | | | F7L5K9 | | |
| | F7LA35; | | | | | |
| | F7LC69 | | | | | |
| Bacteroides ovatus ATCC 8483 | A7LQH9; | | | A7LWH7 | | |
| | A7LRR0; | | | | | |
| | A7M2Q0; | | | | | |
| | A7M2Q3 | | | | | |
| Bacteroides ovatus | I8YEK4; | | | I8Y563 | | |
| CL02T12C04 | I9HKZ9 | | | | | |
| Bacteroides ovatus | I8YCX7; | | | I8ZAA1 | | |
| CL03T12C18 | I8Z236; | | | | | |
| | I9SXN4 | | | | | |
| Bacteroides ovatus SD CC 2a | D4WPV1 | | | D4X2H8 | | |
| Bacteroides ovatus SD CMC 3f | D4WKT0; | | | D4WJL5 | | |
| | D4WKT3 | | | | | |
| Bacteroides plebeius (strain | B5CZ24 | | | B5CVB8 | | |
| DSM 17135/JCM 12973/M2) | | | | | | |
| Bacteroides salanitronis (strain | F0R0V9 | | | F0R050; | | |
| DSM 18170/JCM 13567/ | | | | F0R0W3; | | |
| BL78) | | | | F0R5B2; | | |
| | | | | F0R5V0 | | |
| Bacteroides sp. 1_1_30 | F7M5A4; | | | F7M3Y1 | | |
| | F7M9Y6 | | | | | |
| Bacteroides sp. 1_1_6 | | | | C6IJ10 | | |
| Bacteroides sp. 2_1_16 | | | | D1JQX4 | | |
| Bacteroides sp. 2_1_22 | D0TYG4 | | | D0TKU6 | | |
| Bacteroides sp. 2_1_56FAA | | | | F7LJF7 | | |
| Bacteroides sp. 2_2_4 | C3QNM8; | | | C3QRR1 | | |
| | C3QNN1; | | | | | |
| | C3R1B9; | | | | | |
| | C3R1E1 | | | | | |
| Bacteroides sp. 3_1_23 | D7JY71; | | | D7JXI6; | | |
| | D7K306; | | | D7JXK1 | | |
| | D7K309; | | | | | |
| | D7K488 | | | | | |
| Bacteroides sp. 3_1_33FAA | D1K1L0 | | | D1JZT9 | | |
| Bacteroides sp. 3_1_40A | E5UXU8 | | E5UQD0 | E5UNZ2; | | |
| | | | | E5UPA7; | | |
| | | | | E5UPB3; | | |
| | | | | E5UQC1; | | |
| | | | | E5UQC2; | | |
| | | | | E5UQD1; | | |
| | | | | E5UWF7; | | |
| | | | | E5UWF8; | | |
| | | | | E5UXF4; | | |
| | | | | E5UXM6; | | |
| | | | | E5UXU9; | | |
| | | | | E5UXV0; | | |
| | | | | E5UXX1; | | |
| | | | | E5UZL7 | | |
| Bacteroides sp. 3_2_5 | | | | C6I2C7 | | |
| Bacteroides sp. 4_1_36 | | | | E5V6C2 | | |
| Bacteroides sp. 4_3_47FAA | C6Z2Z2; | | | C3Q1S8 | | |
| | C3PVZ9 | | | | | |
| Bacteroides sp. CAG:1060 | | | | R5BU37; | | |
| | | | | R5BZD9 | | |
| Bacteroides sp. CAG:189 | | | | R5JC12 | | |
| Bacteroides sp. CAG:462 | | | | R7CTY2; | | |
| | | | | R7CZM1; | | |
| | | | | R7D5K0 | | |
| Bacteroides sp. CAG:545 | | | | R5SB97 | | |
| Bacteroides sp. CAG:598 | | | | R5C9H2 | | |
| Bacteroides sp. CAG:633 | | | | R6FHN9; | | |
| | | | | R6FP05; | | |
| | | | | R6FPN5 | | |
| Bacteroides sp. CAG:702 | | | R5TX68 | R5TXR3; | | |
| | | | | R5U1L7; | | |
| | | | | R5UFA9 | | |
| Bacteroides sp. CAG:709 | | | | R6E7T3 | | |
| Bacteroides sp. CAG:770 | | | | R6T4J1 | | |
| Bacteroides sp. CAG:875 | | | R7AV26 | | | |
| Bacteroides sp. D1 | C3QLH5 | | | C3QAI0 | | |
| Bacteroides sp. D2 | E5CCJ5; | | | E5CBI1 | | |
| | E5CCJ8 | | | | | |
| Bacteroides sp. D20 | | | | D2EWT6 | | |
| Bacteroides sp. D22 | D7J7H2; | | | D7IYY4 | | |
| | D7J7H5 | | | | | |
| Bacteroides thetaiotaomicron | | | | Q8A3Q4; | | |
| (strain ATCC 29148/DSM 2079/ | | | | Q8AB47 | | |
| NCTC 10582/E50/VPI-5482) | | | | | | |
| Bacteroides thetaiotaomicron | | | | R7KXJ1 | | |
| CAG:40 | | | | | | |
| Bacteroides uniformis ATCC | | | | A7V0J8 | | |
| 8492 | | | | | | |
| Bacteroides uniformis CAG:3 | | | | R7ES47 | | |
| Bacteroides uniformis | I8ZPJ7 | | | | | |
| CL03T00C23 | | | | | | |
| Bacteroides uniformis | I91Q50 | | | | | |
| CL03T12C37 | | | | | | |
| Bacteroides vulgatus (strain | A6KWG5 | | A6KXP4; | A6KWF5; | | |
| ATCC 8482/DSM 1447/NCTC | | | A6L2B7 | A6KWF6; | | |
| 11154) | | | | A6KWG3; | | |
| | | | | A6KWG4; | | |
| | | | | A6KWN2; | | |
| | | | | A6KWU4; | | |
| | | | | A6KXP3; | | |
| | | | | A6KXQ3; | | |
| | | | | A6KXQ4; | | |
| | | | | A6KXQ5; | | |
| | | | | A6KZ31; | | |
| | | | | A6KZ37; | | |
| | | | | A6KZF0; | | |
| | | | | A6L1A2; | | |
| | | | | A6L1U1; | | |
| | | | | A6L2B6; | | |
| | | | | A6L2H5; | | |
| | | | | A6L2H6; | | |
| | | | | A6L2H7 | | |
| Bacteroides vulgatus CAG:6 | R7NXX7 | | | R7NX35; | | |
| | | | | R7NZN7; | | |
| | | | | R7NZY2; | | |
| | | | | R7P1U8; | | |
| | | | | R7P1Y1; | | |
| | | | | R7P3M4; | | |
| | | | | R7P4T4; | | |
| | | | | R7P696 | | |
| Bacteroides vulgatus | | | | I9A3Y9 | | |
| CL09T03C04 | | | | | | |
| Bacteroides vulgatus PC510 | D4V8M9 | | | D4VCN1 | | |
| Bacteroides xylanisolvens | D6CGY9 | | | | | |
| Bacteroides xylanisolvens | I9AAJ6; | | | I9UTL6 | | |
| CL03T12C04 | I9US13 | | | | | |
| Bacteroides xylanisolvens SD | D4VIN8 | | | D4VGA6 | | |
| CC 1b | | | | | | |
| Bacteroides xylanisolvens XB1A | | | | D6CY86 | | |
| Baudoinia compniacensis | M2MRM8; | M2MT78; | M2M3K3; | M2LR12; | | |
| (strain UAMH 10762) (Angels' | M2MYR0; | M2N2M0 | M2NCY6; | M2MZ71; | | |
| share fungus) | M2N124; | | M2NJ11 | M2N3S7; | | |
| | M2N4K1 | | | M2NM98 | | |
| Beauveria bassiana (strain | | | | J4VWT5 | | |
| ARSEF 2860) (White | | | | | | |
| muscardine disease fungus) | | | | | | |
| (Tritirachium shiotae) | | | | | | |
| Belliella baltica (strain DSM | I3Z665; | | | | | |
| 15883/CIP 108006/LMG | I3Z675; | | | | | |
| 21964/BA134) | I3Z682 | | | | | |
| Beutenbergia cavernae (strain | C5C1P4 | | | C5BX61; | | C5C396 |
| ATCC BAA-8/DSM 12333/ | | | | C5C1C5; | | |
| NBRC 16432) | | | | C5C6L3 | | |
| Bifidobacterium adolescentis | | | | | A1A048 | |
| (strain ATCC 15703/DSM | | | | | | |
| 20083/NCTC 11814/E194a) | | | | | | |
| Bifidobacterium animalis | | | B8DV45 | | | |
| subsp. lactis (strain AD011) | | | | | | |
| Bifidobacterium animalis | | | B2ECI6 | | | |
| subsp. lactis HN019 | | | | | | |
| Bifidobacterium dentium | | | | D2Q6X7; | | |
| (strain ATCC 27534/DSM | | | | D2Q7A7 | | |
| 20436/JCM 1195/Bd1) | | | | | | |
| Bifidobacterium longum subsp. | | | | B7GNV9 | | |
| infantis (strain ATCC 15697/ | | | | | | |
| DSM 20088/JCM 1222/NCTC | | | | | | |
| 11817/S12) | | | | | | |
| Bifidobacterium longum subsp. | | | | D6ZWT5; | | |
| longum (strain JDM301) | | | | D6ZWU6 | | |
| Bipolaris sorghicola | | Q9HEP2; | | | | |
| | | Q9HEP3 | | | | |
| Bispora antennata | | M1G4Y1 | | | | |
| Bispora sp. MEY-1 | D0QF43 | C6FGW6; | | | | |
| | | F2VRZ4 | | | | |
| Botryosphaeria parva (strain | R1FWZ0; | R1GCT8 | | R1EDI8; | | R1ELU7; |
| UCR-NP2) (Grapevine canker | R1G6Y8; | | | R1ERC6; | | R1EQB5; |
| fungus) (Neofusicoccum | R1GC39; | | | R1GAB3; | | R1EZJ9 |
| parvum) | R1GJW3; | | | R1GCR8; | | |
| | R1GMG1 | | | R1GD80; | | |
| | | | | R1GK20; | | |
| | | | | R1GMY4; | | |
| | | | | R1H3P0 | | |
| Botryotinia fuckeliana (Noble | | B3VSG7; | | | | |
| rot fungus) (Botrytis cinerea) | | Q2LMP0 | | | | |
| Botryotinia fuckeliana (strain | M7TN65; | M7U5V1; | | M7TD64; | | M7U9C3 |
| BcDW1) (Noble rot fungus) | M7U9R1 | M7U9J6 | | M7TT70; | | |
| (Botrytis cinerea) | | | | M7TZ84; | | |
| | | | | M7UX14 | | |
| Botryotinia fuckeliana (strain | G2YJF3; | G2XS85; | | G2XZ70; | | G2XR63 |
| T4) (Noble rot fungus) (Botrytis | G2YPE5 | G2XY42; | | G2Y7E2; | | |
| cinerea) | | G2Y450 | | G2Y957; | | |
| | | | | G2YES6 | | |
| Brachybacterium faecium | C7MFS4; | | | | | |
| (strain ATCC 43885/DSM 4810/ | C7MGN0 | | | | | |
| NCIB 9860) | | | | | | |
| Brachypodium distachyon | I1GPN7; | | | | | |
| (Purple false brome) (Trachynia | I1GUC0; | | | | | |
| distachya) | I1GUR3; | | | | | |
| | I1H7I4; | | | | | |
| | I1HBR2; | | | | | |
| | I1HBR3; | | | | | |
| | I1IWJ6 | | | | | |
| Bradyrhizobium japonicum | Q89T09 | | | | | |
| (strain USDA 110) | | | | | | |
| Bradyrhizobium japonicum | G7D9Z1 | | | | | |
| USDA 6 | | | | | | |
| Bradyrhizobium sp. S23321 | I0GDY2 | | | | | |
| Bradyrhizobium sp. WSM1253 | I2QNV7 | | | | | |
| Bradyrhizobium sp. WSM471 | H5YCM8 | | | | | |
| Bradyrhizobium sp. YR681 | J3HXK0 | | | | | |
| Brevibacillus brevis (Bacillus | | G9B9X7; | | | | |
| brevis) | | Q45VU5 | | | | |
| Brevundimonas diminuta 470-4 | L1QJ12 | | | | | |
| Brevundimonas diminuta ATCC | F4QUK1; | | | | | |
| 11568 | F4R049 | | | | | |
| Brevundimonas sp. BAL3 | B4W9K5 | | | | | |
| Brevundimonas subvibrioides | D9QF36 | | | D9QN14; | | |
| (strain ATCC 15264/DSM 4735/ | | | | D9QNK9 | | |
| LMG 14903/NBRC 16000/ | | | | | | |
| CB 81) (Caulobacter | | | | | | |
| subvibrioides) | | | | | | |
| Burkholderia sp. (strain | D5WLQ4 | | | | | |
| CCGE1002) | | | | | | |
| Burkholderia sp. H160 | B5WNC0 | | | B5WBC9 | | |
| Butyrivibrio hungatei | | Q704N8 | | | | |
| Butyrivibrio proteoclasticus | E0RXQ0; | | E0RVY5 | | | |
| (strain ATCC 51982/DSM | E0RYH1; | | | | | |
| 14932/B316) (Clostridium | E0S105; | | | | | |
| proteoclasticum) | E0S155; | | | | | |
| | E0S1Z8; | | | | | |
| | E0S2F5 | | | | | |
| Caldalkalibacillus thermarum | | | | | F5L479 | |
| TA2.A1 | | | | | | |
| Caldanaerobius | J7JXS8 | | | L0E2R8 | | |
| polysaccharolyticus | | | | | | |
| Caldicellulosiruptor bescii | Q59150 | | | | | |
| (Anaerocellum thermophilum) | | | | | | |
| Caldicellulosiruptor bescii | B9MKT7; | B9MLP1 | | B9MMA2; | | B9MMA7 |
| (strain ATCC BAA-1888/DSM | B9MMA3; | | | B9MNB0 | | |
| 6725/Z-1320) (Anaerocellum | B9MMA5; | | | | | |
| thermophilum) | B9MPI1; | | | | | |
| | B9MPZ4 | | | | | |
| Caldicellulosiruptor | E4QDJ6; | | | E4QA31; | | |
| hydrothermalis (strain DSM | E4QEC9 | | | E4QC47; | | |
| 18901/VKM B-2411/108) | | | | E4QC52 | | |
| Caldicellulosiruptor | E4S4K4; | | | | | |
| kristjanssonii (strain ATCC | E4S6E9; | | | | | |
| 700853/DSM 12137/I77R1B) | E4S9X6 | | | | | |
| Caldicellulosiruptor | E4SDC0; | E4SCE2 | | E4SCU0; | | E4SEI1; |
| kronotskyensis (strain DSM | E4SE15; | | | E4SCU6; | | E4SHH9 |
| 18902/VKM B-2412/2002) | E4SGH7; | | | E4SHI4 | | |
| | E4SHI1; | | | | | |
| | E4SHI3; | | | | | |
| | E4SHW9 | | | | | |
| Caldicellulosiruptor | G2PU15; | | | | | |
| lactoaceticus 6A | G2PWE2; | | | | | |
| | G2PXV4 | | | | | |
| Caldicellulosiruptor obsidiansis | D9TFI1; | | | D9TGZ3 | | |
| (strain ATCC BAA-2073/strain | D9TIQ9; | | | | | |
| OB47) | D9TJ43 | | | | | |
| Caldicellulosiruptor owensensis | E4Q2A2; | E4Q1W4 | | E4Q2A1; | | E4Q2A6 |
| (strain ATCC 700167/DSM | E4Q2A4; | | | E4Q6J2; | | |
| 13100/OL) | E4Q2A7; | | | E4Q6K2; | | |
| | E4Q4B5; | | | E4Q6K9 | | |
| | E4Q538; | | | | | |
| | E4Q5G9 | | | | | |
| Caldicellulosiruptor | A4XG17; | | | A4XGG5; | | A4XM46 |
| saccharolyticus (strain ATCC | A4XHD0; | | | A4XJR7 | | |
| 43494/DSM 8903) | A4XIF7; | | | | | |
| | A4XM47; | | | | | |
| | A4XM50; | | | | | |
| | A4XM52 | | | | | |
| Caldicellulosiruptor sp. (strain | P40944 | | | | | |
| Rt8B.4) | | | | | | |
| Caldicellulosiruptor sp. F32 | I7D8W6 | I7DIS4 | | | | |
| Caldicellulosiruptor sp. Rt69B.1 | O52373; | O52375 | | | | |
| | O52374 | | | | | |
| Caldicellulosiruptor sp. Tok7B.1 | Q9AQG2; | | | | | |
| | Q9X3P5; | | | | | |
| | Q9X3P6 | | | | | |
| Caldilinea aerophila (strain | | | | I0I8F1 | | |
| DSM 14535/JCM 11387/ | | | | | | |
| NBRC 104270/STL-6-O1) | | | | | | |
| Caldocellum saccharolyticum | O30421; | | | | | P23552 |
| (Caldicellulosiruptor | O30427; | | | | | |
| saccharolyticus) | P10474; | | | | | |
| | P23556; | | | | | |
| | P23557 | | | | | |
| Calothrix sp. PCC 6303 | | | | | K9V1K2 | |
| Calothrix sp. PCC 7507 | K9PJ14 | | | | K9PK21 | |
| Candidatus Microthrix | | | | R4Z4J2 | | |
| parvicella RN1 | | | | | | |
| Canis familiaris (Dog) (Canis | | | | | | Q01634 |
| lupus familiaris) | | | | | | |
| Capnocytophaga sp. oral taxon | F3Y3V9 | | | F3XWE2 | | |
| 329 str. F0087 | | | | | | |
| Catenulispora acidiphila (strain | C7PW30; | C7PX63; | | C7QAK5 | C7QB30 | |
| DSM 44928/NRRL B-24433/ | C7PYE0; | C7PX75; | | | | |
| NBRC 102108/JCM 14897) | C7Q352; | C7Q365 | | | | |
| | C7Q386 | | | | | |
| Caulobacter crescentus (strain | Q9A404; | | | | | |
| ATCC 19089/CB15) | Q9A4M7 | | | | | |
| Caulobacter crescentus (strain | B8H1R0; | | | | | |
| NA1000/CB15N) | B8H365 | | | | | |
| Caulobacter crescentus OR37 | R0CX88 | | | | | |
| Caulobacter segnis (strain ATCC | D5VIA7 | | | D5VNB5 | | |
| 21756/DSM 7131/JCM 7823/ | | | | | | |
| NBRC 15250/LMG 17158/ | | | | | | |
| TK0059) (Mycoplana segnis) | | | | | | |
| Caulobacter sp. (strain K31) | B0SWF4; | | B0T6Y0 | B0SVS2; | | |
| | B0T4M1 | | | B0SWT8 | | |
| Caulobacter sp. AP07 | J2HM29; | | | | | |
| | J3A2K3 | | | | | |
| Cecembia lonarensis LW9 | K1KYP9 | | | | | |
| Cellulomonas fimi | P07986; | P54865 | | | | |
| | Q3YAW6; | | | | | |
| | Q59277; | | | | | |
| | Q59278 | | | | | |
| Cellulomonas fimi (strain ATCC | F2XFS7; | F4H710 | | F4GZV5; | | F4H8J5 |
| 484/DSM 20113/JCM 1341/ | F4GY46; | | | F4H006; | | |
| NBRC 15513/NCIMB 8980/ | F4GZV4; | | | F4H0R3; | | |
| NCTC 7547) | F4H454; | | | F4H0R8; | | |
| | F4H4N7; | | | F4H6I4; | | |
| | F4H8I0 | | | F4H8J6 | | |
| Cellulomonas flavigena | | A2AWV8; | | | | |
| | | Q14ST6; | | | | |
| | | Q9AG99 | | | | |
| Cellulomonas flavigena (strain | D5UDE7; | D5UGI0; | | D5UBT5 | | |
| ATCC 482/DSM 20109/NCIB | D5UDG3; | D5UGI2 | | | | |
| 8073/NRS 134) | D5UDH4; | | | | | |
| | D5UFC2; | | | | | |
| | D5UFE6; | | | | | |
| | D5UGI1; | | | | | |
| | D5UGW9; | | | | | |
| | D5UH90; | | | | | |
| | D5UI30; | | | | | |
| | D5UIQ1; | | | | | |
| | D5UIQ2; | | | | | |
| | D5UJX7; | | | | | |
| | D5UK72; | | | | | |
| | D5UL25 | | | | | |
| Cellulomonas uda | | | | | P18336 | |
| Cellulophaga algicola (strain | E6X3M9; | | | E6X8Z2 | | |
| DSM 14237/IC166/ACAM | E6X3N2; | | | | | |
| 630) | E6X8P9; | | | | | |
| | E6X9A6 | | | | | |
| Cellulophaga lytica (strain ATCC | | | | F0R9F0 | | |
| 23178/DSM 7489/JCM 8516/ | | | | | | |
| NBRC 14961/NCIMB 1423/ | | | | | | |
| VKM B-1433/Cy I20) | | | | | | |
| Cellulosilyticum lentocellum | F2JL21; | | | | F2JM53 | |
| (strain ATCC 49066/DSM 5427/ | F2JLG8; | | | | | |
| NCIMB 11756/RHM5) | F2JMA0; | | | | | |
| (Clostridium lentocellum) | F2JRS1 | | | | | |
| Cellulosilyticum ruminicola | D2KFJ4; | D2KFL9; | | | | |
| | D2KFL8; | D2KFM0 | | | | |
| | D2KFM1; | | | | | |
| | D2KFM2 | | | | | |
| Cellulosimicrobium sp. HY-12 | B2BZ80 | | | | | |
| Cellulosimicrobium sp. HY-13 | D1GET5 | | | | | |
| Cellvibrio gilvus (strain ATCC | F8A0T7; | F8A6K7 | | F8A2W9; | | |
| 13127/NRRL B-14078) | F8A1V8; | | | F8A358; | | |
| | F8A793; | | | F8A364; | | |
| | F8A7J0; | | | F8A392 | | |
| | F8A7L5; | | | | | |
| | F8A7V7 | | | | | |
| Cellvibrio japonicus | Q59675; | Q8VP72 | | | | |
| | Q9RBZ5 | | | | | |
| Cellvibrio japonicus (strain | B3PC74; | B3PIN0 | B3PEK4 | B3PKP8; | | |
| Ueda107) (Pseudomonas | B3PDA8; | | | P95470 | | |
| fluorescens subsp. cellulosa) | P14768; | | | | | |
| | P23030 | | | | | |
| Cellvibrio mixtus | O68541; | M4T1G3 | | | | |
| | Q59301 | | | | | |
| Cellvibrio sp. BR | I3I5N3; | I3I6Q0 | | I3I4A8; | | |
| | I3I776; | | | I3I4I0; | | |
| | I3I836 | | | I3I6P0; | | |
| | | | | I3I874 | | |
| Ceriporiopsis subvermispora | M2PHP3; | M2QR82 | M2RLR1 | M2QRG4; | | |
| (strain B) (White-rot fungus) | M2QAI7; | | | M2QRX4 | | |
| | M2QEW7; | | | | | |
| | M2QU44; | | | | | |
| | M2QYI8; | | | | | |
| | M2R6Q4 | | | | | |
| Chaetomium cupreum | | Q0GA11 | | | | |
| Chaetomium globosum (strain | Q2GM35; | Q2GN95; | | Q2GM45; | | |
| ATCC 6205/CBS 148.51/DSM | Q2GTY2; | Q2GZ11; | | Q2HCA4 | | |
| 1962/NBRC 6347/NRRL 1970) | Q2GXB6; | Q2GZB2; | | | | |
| (Soil fungus) | Q2H7X4; | Q2H5I3; | | | | |
| | Q2HHK0; | Q2HCI0; | | | | |
| | Q2HIC4 | Q2HCI7; | | | | |
| | | Q2HCS6; | | | | |
| | | Q2HI25 | | | | |
| Chaetomium gracile | | Q12579; | | | | |
| | | Q12580 | | | | |
| Chaetomium sp. CQ31 | | G0WRC8 | | | | |
| Chaetomium thermophilum | | Q06AK8; | | | | |
| | | Q6UN40; | | | | |
| | | Q8J1V4; | | | | |
| | | Q8J1V5; | | | | |
| | | Q8J1V6 | | | | |
| Chaetomium thermophilum | G0S8P5; | G0RYY1; | | | | |
| (strain DSM 1495/CBS 144.50/ | G0S9R7; | G0S9X3; | | | | |
| IMI 039719) | G0SBF1 | G0SBC5 | | | | |
| Chamaesiphon minutus PCC | K9UHG1 | | | | | |
| 6605 | | | | | | |
| Chitinophaga pinensis (strain | C7PGI9 | | C7PNN6; | C7PDM8; | | C7PR29; |
| ATCC 43595/DSM 2588/NCIB | | | C7PNN7 | C7PFM4; | | C7PTZ8 |
| 11800/UQM 2034) | | | | C7PFM4; | | |
| | | | | C7PHN4; | | |
| | | | | C7PKI1; | | |
| | | | | C7PLN9; | | |
| | | | | C7PLQ8; | | |
| | | | | C7PN14; | | |
| | | | | C7PNN1; | | |
| | | | | C7PV31 | | |
| Chroococcidiopsis thermalis | K9U065; | | | | | K9U6Z7 |
| PCC 7203 | K9U0C7 | | | | | |
| Chryseobacterium gleum ATCC | D7W1L4 | | | | | |
| 35910 | | | | | | |
| Chryseobacterium sp. CF314 | J2K6H2 | | | | | |
| Chrysosporium lucknowense | G3FAQ8; | G3FAR1 | | F2X2F4 | | |
| | G3FAQ9 | | | | | |
| Chthoniobacter flavus Ellin428 | B4CV60 | | | | | |
| CLavibacter michiganensis | Q7X3X6 | | | | | |
| subsp. michiganensis | | | | | | |
| CLavibacter michiganensis | A5CM25; | | | | | |
| subsp. michiganensis (strain | A5CRL6; | | | | | |
| NCPPB 382) | A5CRL7 | | | | | |
| CLavibacter michiganensis | M5B788; | | | | | |
| subsp. nebraskensis NCPPB | M5B947; | | | | | |
| 2581 | M5BAQ3 | | | | | |
| CLavibacter michiganensis | B0RHV2 | | | | | |
| subsp. sepedonicus (strain | | | | | | |
| ATCC 33113/JCM 9667) | | | | | | |
| CLaviceps purpurea (Ergot | O74717 | O74716 | | | | |
| fungus) (Sphacelia segetum) | | | | | | |
| CLaviceps purpurea (strain 20.1) | M1WGK0 | M1W9A1 | | | | |
| (Ergot fungus) (Sphacelia | | | | | | |
| segetum) | | | | | | |
| Clostridium acetobutylicum | Q97TI5; | | | | | |
| (strain ATCC 824/DSM 792/ | Q97TP5 | | | | | |
| JCM 1419/LMG 5710/VKM B- | | | | | | |
| 1787) | | | | | | |
| Clostridium acetobutylicum | F0KEF0; | | | | | |
| (strain EA 2018) | F0KEL3 | | | | | |
| Clostridium acetobutylicum | F7ZYH3; | | | | | |
| DSM 1731 | F7ZYN5 | | | | | |
| Clostridium asparagiforme DSM | C0D4Z3 | | | | | |
| 15981 | | | | | | |
| Clostridium beijerinckii (strain | | A6LXV0 | A6M2F3 | | | |
| ATCC 51743/NCIMB 8052) | | | | | | |
| (Clostridium acetobutylicum) | | | | | | |
| Clostridium butyricum 5521 | | | | | B1QSF8 | |
| Clostridium butyricum E4 str. | | | | | C4IIY0 | |
| BoNT E BL5262 | | | | | | |
| Clostridium cellulolyticum | B8I0L1; | B8I371; | | B8I0N8; | B8I0N9; | B8I0P0 |
| (strain ATCC 35319/DSM 5812/ | B8I4I7; | B8I7X1 | | B8I1U1; | B8I0S8; | |
| JCM 6584/H10) | B8I5B9; | | | B8I3H7; | P37699 | |
| | B8I5C0; | | | B8I6V0; | | |
| | Q0PRN5 | | | B8I9B3 | | |
| Clostridium cellulovorans | Q6J286 | Q8GH59 | | | | |
| Clostridium cellulovorans | D9SST3 | D9SP57 | | D9SQB8; | D9SUM5 | |
| (strain ATCC 35296/DSM 3052/ | | | | D9SQS6; | | |
| OCM 3/743B) | | | | D9STE2; | | |
| | | | | D9STF7 | | |
| Clostridium clariflavum (strain | G8LX95; | G8LV53; | | | | |
| DSM 19732/NBRC 101661/ | G8LZ66; | G8LXE2; | | | | |
| EBR45) | G8LZE0; | G8M209 | | | | |
| | G8M1U0; | | | | | |
| | G8M263 | | | | | |
| Clostridium josui | Q9F1V3 | | | | P37701 | |
| Clostridium leptum DSM 753 | A7VWS2 | | | | | |
| Clostridium papyrosolvens DSM | F1T7G6; | F1TF26; | F1TES6 | F1T8P5; | F1T7N5; | F1TBV3 |
| 2782 | F1T879; | F1TIC1 | | F1T8P7; | F1T8Z1; | |
| | F1T880; | | | F1T8S3; | F1TIA5 | |
| | F1T8P4; | | | F1TFD0 | | |
| | F1TCW2; | | | | | |
| | F1TF59 | | | | | |
| Clostridium phytofermentans | A9KJ12; | A9KJ59 | A9KLB5; | A9KIE4; | A9KRR7 | |
| (strain ATCC 700394/DSM | A9KJ62; | | A9KTC7 | A9KJE5; | | |
| 18823/ISDg) | A9KL60; | | | A9KLD2; | | |
| | A9KPY5; | | | A9KMY2; | | |
| | A9KQ55 | | | A9KRB0; | | |
| | | | | A9KTC1 | | |
| Clostridium saccharobutylicum | | P17137 | | | | |
| Clostridium | M1MBH5; | M1LSN7; | | | | |
| saccharoperbutylacetonicum | M1MG09 | M1MVW9 | | | | |
| N1-4(HMT) | | | | | | |
| Clostridium sp. BNL1100 | H2JAY0; | H2J8J3; | | | | |
| | H2JDB7; | H2J8J4; | | | | |
| | H2JG72; | H2JDL3; | | | | |
| | H2JHU1; | H2JIH7 | | | | |
| | H2JHU2 | | | | | |
| Clostridium sp. CAG:1013 | | | | R5A1T2; | | |
| | | | | R5A2C5 | | |
| Clostridium sp. CAG:122 | | | | R5S437 | | |
| Clostridium sp. CAG:167 | | | | R5VJS3; | | |
| | | | | R5WGW4 | | |
| Clostridium sp. CAG:230 | | | R6DGU0 | | | |
| Clostridium sp. CAG:253 | | | | R6M248; | | |
| | | | | R6M9G9 | | |
| Clostridium sp. CAG:413 | | | | | | R6NE49 |
| Clostridium sp. CAG:448 | | | | | | R6T205 |
| Clostridium sp. CAG:62 | R7C4U4 | | | R7C6Y4; | R7C7X3 | |
| | | | | R7C7M1; | | |
| | | | | R7C8Y5 | | |
| Clostridium sp. CAG:91 | | | | R6VSF7 | | |
| Clostridium sp. DL-VIII | G7M400 | G7M8A3 | | G7M201 | G7M206 | |
| Clostridium sp. Maddingley | K6SWI9 | K6TUI5 | | | | |
| MBC34-26 | | | | | | |
| Clostridium stercorarium | P40942; | P33558; | | P48790 | | |
| | Q8GJ37; | Q8GJ44 | | | | |
| | Q8GJ38; | | | | | |
| | Q9XDV5 | | | | | |
| Clostridium stercorarium | L7VI53; | L7VQD8 | | L7VNS7 | | |
| subsp. stercorarium (strain | L7VLT8; | | | | | |
| ATCC 35414/DSM 8532/ | L7VM99 | | | | | |
| NCIMB 11754) | | | | | | |
| Clostridium termitidis CT1112 | | | | | S0FT90 | |
| Clostridium thermocellum | O32374; | | | | P0C2S2 | |
| | P38535; | | | | | |
| | P51584; | | | | | |
| | Q70DK4 | | | | | |
| Clostridium thermocellum | A3DDW7; | A3DJP0 | | A3DHB3; | A3DC29 | |
| (strain ATCC 27405/DSM | A3DDW7; | | | A3DHG9 | | |
| 1237) | A3DGI0; | | | | | |
| | A3DGI0; | | | | | |
| | A3DH97; | | | | | |
| | A3DIL1; | | | | | |
| | A3DIL1; | | | | | |
| | P10478 | | | | | |
| Clostridium thermocellum | E6UPX5; | E6UTI4; | | E6USN6; | E6ULX8 | |
| (strain DSM 1313/LMG 6656/ | E6UPX5; | E6UTI5 | | E6USU7; | | |
| LQ8) | E6UQ43; | | | E6UT95 | | |
| | E6UQB4; | | | | | |
| | E6UR90; | | | | | |
| | E6US71 | | | | | |
| Clostridium thermocellum AD2 | H8EAW3; | H8EIA0 | | H8EAY0; | H8EBK2 | |
| | H8ECS9; | | | H8EB42; | | |
| | H8EF39; | | | H8EB45 | | |
| | H8EFD3; | | | | | |
| | H8EHK9 | | | | | |
| Clostridium thermocellum DSM | C7HBR7; | C7HJV5 | | C7HDU9 | C7HGK4 | |
| 2360 | C7HDW6; | | | | | |
| | C7HEZ0; | | | | | |
| | C7HH50; | | | | | |
| | C7HI91 | | | | | |
| Clostridium thermocellum | D1NIL9; | D1NR31 | | D1NNT4; | D1NLD2 | |
| JW20 | D1NPG8; | | | D1NNT7 | | |
| | D1NPL0; | | | | | |
| | D1NPW2; | | | | | |
| | D1NQA4 | | | | | |
| Clostridium thermocellum YS | H8EJX7; | H8ERL6; | | H8EQS5; | H8EM30 | |
| | H8ENN8; | H8ERL7; | | H8EQS8; | | |
| | H8ENZ5; | H8ES66 | | H8EQZ0 | | |
| | H8EPS5; | | | | | |
| | H8ER07 | | | | | |
| Coccidioides immitis (strain RS) | | | | J3KLN1 | | |
| (Valley fever fungus) | | | | | | |
| Coccidioides posadasii (strain | | | | C5P382 | | |
| C735) (Valley fever fungus) | | | | | | |
| Coccidioides posadasii (strain | | | | E9CR16 | | |
| RMSCC 757/Silveira) (Valley | | | | | | |
| fever fungus) | | | | | | |
| Cochliobolus carbonum | Q6GXE5 | Q00350; | | | | |
| (Bipolaris zeicola) | | Q00351; | | | | |
| | | Q06562 | | | | |
| Cochliobolus heterostrophus | | Q9HDL7; | | | | |
| (Southern corn leaf blight | | Q9HEN7 | | | | |
| fungus) (Bipolaris maydis) | | | | | | |
| Cochliobolus heterostrophus | N4WUR0; | N4WMV3; | N4XI75; | N4WG93; | | N4XE05 |
| (strain C4/ATCC 48331/race | N4WYQ3; | N4WQA7; | N4XRQ1 | N4WGT7; | | |
| T) (Southern corn leaf blight | N4XE16; | N4WYY8; | | N4WYN7; | | |
| fungus) (Bipolaris maydis) | N4XH14; | N4XPF6; | | N4X598; | | |
| | N4XSQ9 | N4XW83 | | N4X685; | | |
| | | | | N4X8G6; | | |
| | | | | N4X9J0; | | |
| | | | | N4XBX8; | | |
| | | | | N4XFI2; | | |
| | | | | N4XHF0; | | |
| | | | | N4XL58; | | |
| | | | | N4XN38; | | |
| | | | | N4XNV7 | | |
| Cochliobolus heterostrophus | M2TUZ8; | M2TBN8; | M2TX43; | M2SIQ5; | | M2UCN8 |
| (strain C5/ATCC 48332/race | M2UBD0; | M2UFM9; | M2UWF6 | M2SP90; | | |
| O) (Southern corn leaf blight | M2UBJ4; | M2UQB2; | | M2U0R6; | | |
| fungus) (Bipolaris maydis) | M2UHU2; | M2UYY3; | | M2U193; | | |
| | M2UXD7 | M2V3W7 | | M2U3E2; | | |
| | | | | M2U3Y4; | | |
| | | | | M2UAQ1; | | |
| | | | | M2UEM4; | | |
| | | | | M2UH67; | | |
| | | | | M2UIG4; | | |
| | | | | M2UK73; | | |
| | | | | M2UQA8; | | |
| | | | | M2UTX1; | | |
| | | | | M2UYE8; | | |
| | | | | M2V1B2 | | |
| Cochliobolus sativus (Common | | O13447; | | | | |
| root rot and spot blotch | | Q9HEN5; | | | | |
| fungus) (Bipolaris sorokiniana) | | Q9HEN6 | | | | |
| Cochliobolus sativus (strain | M2RL72; | M2RAB4; | M2TCS7; | M2QVP1; | | M2SW94 |
| ND90Pr/ATCC 201652) | M2RW02; | M2RSH5; | M2TMS4 | M2REZ1; | | |
| (Common root rot and spot | M2SAN0; | M2SPH1; | | M2RI73; | | |
| blotch fungus) (Bipolaris | M2SV83; | M2TAK5; | | M2RKL6; | | |
| sorokiniana) | M2SYV3 | M2TFB6 | | M2RWN7; | | |
| | | | | M2S8J2; | | |
| | | | | M2SL82; | | |
| | | | | M2SP05; | | |
| | | | | M2SQF9; | | |
| | | | | M2SVA0; | | |
| | | | | M2T058; | | |
| | | | | M2T4C6 | | |
| Cohnella laevoribosii | D5KTJ5 | D5KTJ4 | | | | |
| Colletotrichum gloeosporioides | L2FEH5; | L2FB85; | | L2FFQ3; | | |
| (strain Nara gc5) (Anthracnose | L2FHT9; | L2FC37; | | L2FQ59; | | |
| fungus) (Glomerella cingulata) | L2FLQ3; | L2FX67; | | L2FT14; | | |
| | L2FVC8; | L2GGU6 | | L2G0G5; | | |
| | L2G041; | | | L2G1B1; | | |
| | L2GB97; | | | L2GD22; | | |
| | L2GIL4 | | | L2GIN1 | | |
| Colletotrichum graminicola | B5WY69 | | | | | |
| (Maize anthracnose fungus) | | | | | | |
| (Glomerella graminicola) | | | | | | |
| Colletotrichum graminicola | E3Q8L2; | E3Q8W7; | | E3QTC7 | | |
| (strain M1.001/M2/FGSC | E3QLA4; | E3Q964; | | | | |
| 10212) (Maize anthracnose | E3QPW0; | E3QH42; | | | | |
| fungus) (Glomerella | E3QQ57; | E3QVD0 | | | | |
| graminicola) | E3QQ83; | | | | | |
| | E3QSE3; | | | | | |
| | E3QSI4; | | | | | |
| | E3QTE3; | | | | | |
| | E3QWX4 | | | | | |
| Colletotrichum higginsianum | H1V1P3; | H1UW78; | | H1V664; | | |
| (strain IMI 349063) (Crucifer | H1VH43; | H1VIL4; | | H1VJ58; | | |
| anthracnose fungus) | H1VI16; | H1VMM1; | | H1VWE7 | | |
| | H1VIS6; | H1VZ08; | | | | |
| | H1VLH1; | H1W3C8 | | | | |
| | H1VRD9; | | | | | |
| | H1VW86 | | | | | |
| Colletotrichum orbiculare | N4V3B6; | N4V774; | | N4US67; | | |
| (strain 104-T/ATCC 96160/ | N4V5J4; | N4VFY8 | | N4VRX3 | | |
| CBS 514.97/LARS 414/MAFF | N4V5T0; | | | | | |
| 240422) (Cucumber | N4VDC9; | | | | | |
| anthracnose fungus) | N4VH31; | | | | | |
| (Colletotrichum lagenarium | S2CPU9 | | | | | |
| Klebsiella pneumoniae | | | | S2D2I0; | S2E9V6 | |
| 540_1460 | | | | S2DFD3 | | |
| Okayama-7/130/ATCC MYA- | A8N540; | A8NW94; | | | | |
| 4618/FGSC 9003) (Inky cap | A8NBS6; | A8NZY3; | | | | |
| fungus) (Hormographiella | A8NBS7; | A8P8F0; | | | | |
| aspergillata) | A8P570 | A8PG06 | | | | |
| Coprobacillus sp. CAG:826 | | | | R7DP76; | | |
| | | | | R7DRL1; | | |
| | | | | R7DWY4 | | |
| Coraliomargarita akajimensis | | | | D5EQ86; | | |
| (strain DSM 45221/IAM 15411/ | | | | D5ER07 | | |
| JCM 23193/KCTC 12865) | | | | | | |
| Coraliomargarita sp. CAG:312 | R7LCJ6; | | | R7L7Y1 | | |
| | R7LEV1 | | | | | |
| Coriobacterium glomerans | | | | F2NA39 | | |
| (strain ATCC 49209/DSM | | | | | | |
| 20642/JCM 10262/PW2) | | | | | | |
| Crinalium epipsammum PCC | | | | K9VYA8 | | |
| 9333 | | | | | | |
| Cryptococcus adeliensis | O13436 | | | | | |
| Cryptococcus albidus | P07529 | | | | | |
| (Filobasidium floriforme) | | | | | | |
| Cryptococcus flavus | | B0FIU1 | | | | |
| Cryptococcus sp. S-2 | | Q92397 | | | | |
| Cryptovalsa sp. BCC 7197 | Q5XQ46 | | | | | |
| Cupriavidus taiwanensis (strain | | | | | B2AI90 | |
| R1/LMG 19424) (Ralstonia | | | | | | |
| taiwanensis (strain LMG | | | | | | |
| 19424)) | | | | | | |
| Curvularia spicifera | | Q9HEN3; | | | | |
| | | Q9HEN4 | | | | |
| Cyanobium gracile (strain ATCC | K9P9I3 | | | | | |
| 27147/PCC 6307) | | | | | | |
| Cyanothece sp. (strain PCC | B8HLM8 | | | B8HTF9 | | |
| 7425/ATCC 29141) | | | | | | |
| Cyanothece sp. (strain PCC | E0UIA1 | | | E0ULI1 | | |
| 7822) | | | | | | |
| Cyanothece sp. (strain PCC | B7K395 | | | | | |
| 8801) (Synechococcus sp. | | | | | | |
| (strain PCC 8801/RF-1)) | | | | | | |
| Cyanothece sp. (strain PCC | C7QU45 | | | | | |
| 8802) (Synechococcus sp. | | | | | | |
| (strain RF-2)) | | | | | | |
| Cyanothece sp. CCY0110 | A3IKY8 | | | | | |
| Cyclobacterium marinum | G0J4D2 | | | | | |
| (strain ATCC 25205/DSM 745) | | | | | | |
| (Flectobacillus marinus) | | | | | | |
| Cystobacter fuscus DSM 2262 | L9K044; | L9JND2 | | L9JK77; | | |
| | L9KBW1 | | | L9KEL8 | | |
| Cytophaga hutchinsonii (strain | Q11T96; | Q11SH7 | | Q11TF7; | Q11NQ3; | |
| ATCC 33406/NCIMB 9469) | Q11TF8; | | | Q11TG0 | Q11PI8; | |
| | Q11VQ5 | | | | Q11R64; | |
| | | | | | Q11VQ4; | |
| | | | | | Q11W64 | |
| Dacryopinax sp. (strain DJM | M5FXR6; | | M5FYJ4 | M5FT57 | | |
| 731) (Brown rot fungus) | M5G428 | | | | | |
| Deinococcus deserti (strain | | | | C1CZ22; | | |
| VCD115/DSM 17065/LMG | | | | C1CZ23 | | |
| 22923) | | | | | | |
| Deinococcus geothermalis | | | | Q1J2X8; | Q1J317 | |
| (strain DSM 11300) | | | | Q1J2X9 | | |
| Deinococcus gobiensis (strain | | | | H8GXG1; | | |
| DSM 21396/JCM 16679/ | | | | H8GXG2 | | |
| CGMCC1.7299/I-0) | | | | | | |
| Deinococcus maricopensis | | | | E8U3D3; | | |
| (strain DSM 21211/LMG | | | | E8U472; | | |
| 22137/NRRL B-23946/LB-34) | | | | E8U475; | | |
| | | | | E8U4T6; | | |
| | | | | E8U4X0; | | |
| | | | | E8U4X4 | | |
| Deinococcus peraridilitoris | | | | K9ZXM1; | | |
| (strain DSM 19664/LMG | | | | K9ZZI2 | | |
| 22246/CIP 109416/KR-200) | | | | | | |
| Demequina sp. JK4 | B9VSZ3 | | | | | |
| Desulfobacca acetoxidans | | | | | F2NEU8 | |
| (strain ATCC 700848/DSM | | | | | | |
| 11109/ASRB2) | | | | | | |
| Dichomitus squalens (strain | | | | R7SVT9 | | |
| LYAD-421) (Western red white- | | | | | | |
| rot fungus) | | | | | | |
| Dickeya dadantii (strain 3937) | | | | | P27032 | |
| (Erwinia chrysanthemi (strain | | | | | | |
| 3937)) | | | | | | |
| Dickeya zeae (strain Ech1591) | | | | C6CEF3; | | |
| | | | | C6CIS2 | | |
| Dictyoglomus sp. (strain B4A) | P80717; | | | | | |
| | P80718 | | | | | |
| Dictyoglomus thermophilum | Q12603 | P77853 | | | | |
| Dictyoglomus thermophilum | B5YA84 | B5YCB5 | | B5YAH2 | | |
| (strain ATCC 35947/DSM 3960/ | | | | | | |
| H-6-12) | | | | | | |
| Dictyoglomus turgidum (strain | B8E346; | B8E1P4 | | B8E3C7 | | |
| Z-1310/DSM 6724) | B8E3B3 | | | | | |
| Dictyostelium fasciculatum | | | F4PTD1 | | | |
| (strain SH3) (Slime mold) | | | | | | |
| Didymella pisi | | Q00263 | | | | |
| Dyadobacter fermentans | C6W283 | | C6VRM9 | C6VRP2; | | C6VT98 |
| (strain ATCC 700827/DSM | | | | C6VRQ4; | | |
| 18053/NS114) | | | | C6VRR6; | | |
| | | | | C6VWZ1; | | |
| | | | | C6W131; | | |
| | | | | C6W155; | | |
| | | | | C6W1E9; | | |
| | | | | C6W2B0; | | |
| | | | | C6W4T0 | | |
| Dysgonomonas gadei ATCC | F5IWT0; | F5IWT2 | | F5IUN3 | | |
| BAA-286 | F5IX65 | | | | | |
| Dysgonomonas mossii DSM | F8X1L4; | | | F8X4W6 | | |
| 22836 | F8X1N7 | | | | | |
| Echinicola vietnamensis (strain | L0G017; | | | L0FS89; | | |
| DSM 17526/LMG 23754/ | L0G036; | | | L0G2F5 | | |
| KMM 6221) | L0G0S0 | | | | | |
| Ectocarpus siliculosus (Brown | D8LGR5 | | | | | |
| alga) | | | | | | |
| Emericella nidulans (strain | Q00177; | P55332; | | Q5AUM3; | | |
| FGSC A4/ATCC 38163/CBS | Q5BAS4 | P55333 | | Q5AZC8; | | |
| 112.46/NRRL 194/M139) | | | | Q5B8T6; | | |
| (Aspergillus nidulans) | | | | Q5BA96 | | |
| Emiliania huxleyi CCMP1516 | R1BWA1; | | | | | |
| | R1FM39 | | | | | |
| Emticicia oligotrophica (strain | I2EX07; | | I2ERI6 | I2ERB1; | | I2EW11 |
| DSM 17448/GPTSA100-15) | I2EXT0 | | | I2EUN2; | | |
| | | | | I2EXS8; | | |
| | | | | I2F157; | | |
| | | | | I2F158 | | |
| Enterobacter asburiae (strain | | | | G2S6T9 | G2S4H1; | |
| LF7a) | | | | | G2S4H9 | |
| Enterococcus casseliflavus EC10 | | | | C9CJJ2; | | |
| | | | | C9AW69 | | |
| Enterococcus faecium E1636 | | | | D4R8U3; | | |
| | | | | D4R8U6 | | |
| Enterococcus sp. C1 | | | | J0XLG1 | | |
| Epidinium caudatum | Q86S91 | | | | | |
| Epidinium ecaudatum | B7FBK4; | B7FBK8 | | | | |
| | B7FBK5; | | | | | |
| | B7FBK6 | | | | | |
| Escherichia coli (strain K12) | | | | P77713 | P37651 | |
| Escherichia coli E1167 | | | | | E9W7K2 | |
| Escherichia coli E1520 | | | | E9WL45 | E9WM09 | |
| Escherichia coli E482 | | | | | E9X088 | |
| Escherichia coli EC1865 | | | | | K3QL09 | |
| Escherichia coli H120 | | | | | E9XD78 | |
| Escherichia coli H252 | | | | | E9VE61 | |
| Escherichia coli H263 | | | | | E9VUD3 | |
| Escherichia coli H489 | | | | | E9Y6Y0 | |
| Escherichia coli M863 | | | | | E9YVR0 | |
| Escherichia coli O157:H7 | | | | | Q8X5L9 | |
| Escherichia coli TA007 | | | | | E9YG28 | |
| Escherichia coli TW10509 | | | | | E9XSS4 | |
| Escherichia fergusonii B253 | | | | | E9ZCX4 | |
| Ethanoligenens harbinense | | | | | E6U3F5 | |
| (strain DSM 18485/JCM 12961/ | | | | | | |
| CGMCC 1.5033/YUAN-3) | | | | | | |
| Eubacterium cellulosolvens 6 | I5AQ84; | | | | | |
| | I5ARE6; | | | | | |
| | I5ATA1; | | | | | |
| | I5AVN7 | | | | | |
| Eubacterium eligens (strain | | | | C4Z068; | | |
| ATCC 27750/VPI C15-48) | | | | C4Z2I3; | | |
| | | | | C4Z358 | | |
| Eubacterium eligens CAG:72 | | | | R5ZHQ2; | | |
| | | | | R5ZY75 | | |
| Eubacterium rectale (strain | | | | C4ZGA3 | | |
| ATCC 33656/VPI 0990) | | | | | | |
| Eubacterium ruminantium | Q47871 | | | | | |
| Eubacterium sp. CAG:248 | | | | R6K6S8; | | |
| | | | | R6KAW3 | | |
| Eubacterium sp. CAG:252 | | | | R6K338; | | |
| | | | | R6L0S4 | | |
| Eubacterium sp. CAG:274 | | | | R6PAZ1 | | |
| Eubacterium sp. CAG:38 | | | | R7HDS6 | | |
| Eubacterium sp. CAG:76 | | | | R7NAC5; | | |
| | | | | R7NGB2 | | |
| Eubacterium sp. CAG:86 | | | | R5E0X1; | | |
| | | | | R5E7P7 | | |
| Eucalyptus globulus subsp. | I0IK83; | | | | | |
| globulus (Tasmanian blue gum) | Q27U87 | | | | | |
| Eucalyptus pilularis | I0IK81 | | | | | |
| Eucalyptus pyrocarpa | I0IK82 | | | | | |
| Eudiplodinium maggii | B7FBK7 | | | | | |
| Eutypa lata (strain UCR-EL1) | M7S6D5; | M7STD0; | M7T8N6 | M7SQF4; | | |
| (Grapevine dieback disease | M7TCX0; | M7SU57 | | M7SR21; | | |
| fungus) (Eutypa armeniacae) | M7TKW8; | | | M7STH9; | | |
| | M7TYC2 | | | M7SUQ1; | | |
| | | | | M7T504; | | |
| | | | | M7T951; | | |
| | | | | M7TDX5; | | |
| | | | | M7TED5; | | |
| | | | | M7TPM2; | | |
| | | | | M7TTE5; | | |
| | | | | M7TTY2; | | |
| | | | | M7TZS9 | | |
| Exophiala dermatitidis (strain | H6BQ88 | | | | | |
| ATCC 34100/CBS 525.76/ | | | | | | |
| NIH/UT8656) (Black yeast) | | | | | | |
| (Wangiella dermatitidis) | | | | | | |
| Faecalibacterium sp. CAG:74 | | | | R7I5Q6 | R7I835 | |
| Fibrella aestuarina BUZ 2 | I0K883; | | | I0K894; | I0K886 | I0K891 |
| | I0K8A3; | | | I0K897; | | |
| | I0K8D6; | | | I0K9G9; | | |
| | I0KB42; | | | I0KB36; | | |
| | I0KDV5 | | | I0KEX6 | | |
| Fibrisoma limi BUZ 3 | I2GCZ9; | | | I2GBU3; | I2GCZ5 | I2GCY9 |
| | I2GD64; | | | I2GCY5; | | |
| | I2GHZ0; | | | I2GDK4; | | |
| | I2GLV6; | | | I2GHD7; | | |
| | I2GQ21 | | | I2GHU5; | | |
| | | | | I2GKA6; | | |
| | | | | I2GRC6 | | |
| Fibrobacter succinogenes | A7UG54; | C9RK54; | | C9RIW4; | A7UG68; | |
| (strain ATCC 19169/S85) | C9RKU1; | C9RLL3; | | C9RIW5; | C9RJV6; | |
| | C9RMY6; | P35811 | | C9RIW6; | C9RJZ0; | |
| | C9RMY9; | | | C9RMH3; | C9RLD6; | |
| | C9RS51; | | | C9RMH4; | C9RMD2; | |
| | D9S458; | | | C9RMH5; | C9RQI6; | |
| | D9S9N9; | | | C9RP41; | C9RS59 | |
| | Q9F107; | | | C9RS19; | | |
| | Q9F108; | | | C9RS32 | | |
| | Q9F109; | | | | | |
| | Q9F4K9; | | | | | |
| | Q9F4L0 | | | | | |
| Fibroporia radiculosa (strain | J4G2H9; | | | J4GMZ4; | | |
| TFFH 294) (Brown rot fungus) | J4GN24; | | | J41948 | | |
| (Antrodia radiculosa) | J4HVE1 | | | | | |
| Firmicutes bacterium CAG:212 | | | | | | R5YD38 |
| Firmicutes bacterium CAG:227 | | | | R6V8L8; | | |
| | | | | R6V8M5 | | |
| Firmicutes bacterium CAG:272 | | | | R6TMP0; | | R6TM44; |
| | | | | R6TW88; | | R6U9F0 |
| | | | | R6UM92 | | |
| Firmicutes bacterium CAG:345 | | | | R6XUF1; | | |
| | | | | R6Y1Z3 | | |
| Firmicutes bacterium CAG:424 | | | | R6SCT5; | | |
| | | | | R6SCU6 | | |
| Firmicutes bacterium CAG:449 | | | | R6R0J0; | | |
| | | | | R6R8V4; | | |
| | | | | R6RCU1; | | |
| | | | | R6S7I8 | | |
| Firmicutes bacterium CAG:534 | | | | | R6ZW88 | |
| Firmicutes bacterium CAG:65 | | | | R6EM07; | | |
| | | | | R6EXJ1 | | |
| Firmicutes bacterium CAG:882 | | | | R7BG44; | | |
| | | | | R7BJY9; | | |
| | | | | R7BK21 | | |
| Firmicutes bacterium CAG:95 | | | | R7N3W7 | R7N6S9 | |
| Fischerella sp. JSC-11 | G6FP94; | | | | | |
| | G6FQY7 | | | | | |
| Flammulina velutipes (Agaricus | G8A553 | | | | | |
| velutipes) | | | | | | |
| Flavobacteria bacterium BAL38 | | A3J750 | | | | |
| Flavobacteriaceae bacterium | C6X163 | | | | | |
| (strain 3519-10) | | | | | | |
| Flavobacterium | | | G2Z0K3; | | | |
| branchiophilum (strain FL-15) | | | G2Z797 | | | |
| Flavobacterium johnsoniae | A5FD49; | | A5FJM0; | A5FC13; | A5FD37; | A5FLV4 |
| (strain ATCC 17061/DSM 2064/ | A5FI54; | | A5FJM1; | A5FCH5; | A5FL64 | |
| UW101) (Cytophaga | A5FIE5 | | A5FJM4 | A5FD23; | | |
| johnsonae) | | | | A5FD31; | | |
| | | | | A5FE30; | | |
| | | | | A5FFA0; | | |
| | | | | A5FIA6; | | |
| | | | | A5FIB4; | | |
| | | | | A5FIB6; | | |
| | | | | A5FIE7 | | |
| Flavobacterium sp. CF136 | J2J5P0; | J2JRQ2 | | J2J4N9; | | |
| | J2JB53; | | | J2J4P3 | | |
| | J2JW93 | | | | | |
| Flavobacterium sp. F52 | J0RSR2; | | | | | |
| | J1AM95 | | | | | |
| Flavobacterium sp. LW53 | C0M1B6 | | | | | |
| Flavobacterium sp. MSY2 | Q288H9 | | | | | |
| Frankia sp. (strain Ccl3) | | | | Q2J5W6 | | |
| Frankia sp. (strain EAN1pec) | | | | A8L9G2; | | |
| | | | | A8LEI6; | | |
| | | | | A8LGF7 | | |
| Fulvimarina pelagi HTCC2506 | Q0G548 | | | | | |
| Fusarium oxysporum (Fusarium | P46239; | | | | | |
| vascular wilt) | Q8TFC1; | | | | | |
| | Q8TGC2; | | | | | |
| | Q8TGC3 | | | | | |
| Fusarium oxysporum (strain | F9F6U4; | F9F5R3; | | F9G6T0 | | |
| Fo5176) (Fusarium vascular | F9F9C7; | F9FIS6; | | | | |
| wilt) | F9FSV2 | F9FP27 | | | | |
| Fusarium oxysporum f. sp. | N4TV99; | N4TI83; | | N4TU80 | | |
| cubense (strain race 1) | N4U098; | N4UAR1; | | | | |
| (Panama disease fungus) | N4UPR9; | N4UIS2 | | | | |
| | N4UTG6; | | | | | |
| | N4UXB3 | | | | | |
| Fusarium oxysporum f. sp. | N1RMI9; | N1RLQ5; | | N1S2J3 | | |
| cubense (strain race 4) | N1RT99; | N1S0D4; | | | | |
| (Panama disease fungus) | N1RZZ3; | N1S850 | | | | |
| | N1S2Q7 | | | | | |
| Fusarium oxysporum f. sp. | O59937; | Q9C1R1; | | | | |
| lycopersici | O59938; | Q9C1R2 | | | | |
| | O93976 | | | | | |
| Fusarium oxysporum f. sp. | B3A0S5; | J9MMM6; | | | | |
| lycopersici (strain 4287/CBS | J9MQ16; | J9N379; | | | | |
| 123668/FGSC 9935/NRRL | J9NDZ1; | J9NKL5 | | | | |
| 34936) (Fusarium vascular wilt | J9NH29; | | | | | |
| of tomato) | J9NQE9 | | | | | |
| Fusarium pseudograminearum | K3VBK3; | K3UXI6; | | K3VU79 | | |
| (strain CS3096) (Wheat and | K3VD03; | K3VKV9; | | | | |
| barley crown-rot fungus) | K3VEU9; | K3VRV5 | | | | |
| | K3VLQ8; | | | | | |
| | K3VYX6 | | | | | |
| Gaeumannomyces graminis | | Q9UVZ4 | | | | |
| var. avenae | | | | | | |
| Gaeumannomyces graminis | J3NMP6; | J3NLQ4; | | J3NSD9 | | |
| var. tritici (strain R3-111a-1) | J3NPT0; | J3NW75; | | | | |
| (Wheat and barley take-all root | J3NS10; | J3PI48 | | | | |
| rot fungus) | J3NZ13; | | | | | |
| | J3PH00; | | | | | |
| | J3PH11; | | | | | |
| | J3PHV0; | | | | | |
| | J3PHY0 | | | | | |
| Galbibacter sp. ck-I2-15 | K2P2D1; | | | | | |
| | K2QL53 | | | | | |
| Gallaecimonas xiamenensis 3- | | | | K2JPC1 | | |
| C-1 | | | | | | |
| Gamma proteobacterium | Q1YTG9 | | | | | |
| HTCC2207 | | | | | | |
| Geobacillus sp. (strain C56-T3) | D7D6B5; | | | D7D512; | | |
| | D7D6C8 | | | D7D513 | | |
| Geobacillus sp. (strain | D3EE78; | D3EJU7 | | D3E9F2; | | |
| Y412MC10) | D3EGF1; | | | D3EAN5; | | |
| | D3EH13; | | | D3EBL1; | | |
| | D3EH14 | | | D3ED47; | | |
| | | | | D3EH12; | | |
| | | | | D3EJX3 | | |
| Geobacillus sp. (strain | E8SUS8; | | | E8SVB3; | | E8SUT6 |
| Y412MC52) | E8SV95 | | | E8SVB7 | | |
| Geobacillus sp. (strain | C9RT34; | | | C9RT69 | | C9RT42 |
| Y412MC61) | C9RT47 | | | | | |
| Geobacillus sp. 71 | G3G7L3 | | | | | |
| Geobacillus sp. G11MC16 | B4BMD4; | | | | | |
| | B4BME8 | | | | | |
| Geobacillus sp. GHH01 | L7ZSH9; | | | | | |
| | L7ZXR7 | | | | | |
| Geobacillus sp. TC-W7 | D0EM78 | | | | | |
| Geobacillus sp. WBI | B5M201 | | | | | |
| Geobacillus | L7XJX2; | P45705 | | B3EYM8 | | Q9ZFM2 |
| stearothermophilus (Bacillus | P40943; | | | | | |
| stearothermophilus) | P45703; | | | | | |
| | Q09LY9; | | | | | |
| | Q3YBZ9 | | | | | |
| Geobacillus thermantarcticus | F8SUS3 | | | | | |
| Geobacillus | A4IP71; | | | | | |
| thermodenitrificans (strain | A4IP84 | | | | | |
| NG80-2) | | | | | | |
| Geobacillus | F8CSW8; | | | | | |
| thermoglucosidasius (strain | F8CSY1 | | | | | |
| C56-YS93) | | | | | | |
| Geobacillus thermoleovorans | G9IJ64 | | | | | |
| (Bacillus thermoleovorans) | | | | | | |
| Geodermatophilus obscurus | | | D2SC74 | | D2S404; | |
| (strain ATCC 25078/DSM | | | | | D2S408 | |
| 43160/JCM 3152/G-20) | | | | | | |
| Geomyces destructans (strain | L8FQY9; | | | | | |
| ATCC MYA-4855/20631-21) | L8G611 | | | | | |
| (Bat white-nose syndrome | | | | | | |
| fungus) | | | | | | |
| Gibberella zeae (strain PH-1/ | I1RLP3; | I1RII8; | | I1RGX1 | | |
| ATCC MYA-4620/FGSC 9075/ | I1RQU5; | I1S2K3 | | | | |
| NRRL 31084) (Wheat head | I1S117; | | | | | |
| blight fungus) (Fusarium | I1S3C6; | | | | | |
| graminearum) | I1S3T9 | | | | | |
| Gibberella zeae (Wheat head | A4UVN0; | Q49SA5; | | | | |
| blight fungus) (Fusarium | Q3ZM13; | Q5NDZ1; | | | | |
| graminearum) | Q49SA1; | Q7ZA57 | | | | |
| | Q49SA4 | | | | | |
| Gillisia limnaea DSM 15749 | | | | H2BRN6; | | |
| | | | | H2BRN8; | | |
| | | | | H2BRN9 | | |
| Glaciecola agarilytica NO2 | K6XA16 | | | | | |
| Glaciecola arctica BSs20135 | K6YC73 | | | K6ZF58 | | |
| Glaciecola chathamensis S18K6 | K6YLV9 | | | | | |
| Glaciecola lipolytica E3 | | | | K6YCW0; | | |
| | | | | K6YE92; | | |
| | | | | K6YEA1 | | |
| Glaciecola mesophila | C0LK93 | | | | | |
| Glaciecola mesophila KMM 241 | K6XVM2 | | | | | |
| Glaciecola polaris LMG 21857 | K7A0W0 | | | | | |
| Glaciecola sp. (strain 4H-3- | F4AKG1 | | | F4ASX1 | F4ARK1 | |
| 7 + YE-5) | | | | | | |
| Glarea lozoyensis (strain ATCC | H0EEW9; | H0EXY5 | | H0EQF3 | | |
| 74030/MF5533) | H0EHV0; | | | | | |
| | H0EMM8; | | | | | |
| | H0EPH7; | | | | | |
| | H0EQY4; | | | | | |
| | H0EWL0; | | | | | |
| | H0EWW8 | | | | | |
| Gloeocapsa sp. PCC 7428 | K9XG05; | | | | K9XH97 | |
| | K9XKD2 | | | | | |
| Gloeophyllum trabeum (Brown | F8T944; | | | | | |
| rot fungus) | P84195 | | | | | |
| Gluconacetobacter hansenii | | | | | P37696 | |
| (Acetobacter hansenii) | | | | | | |
| Gordonia sp. NB4-1Y | | | | M7A435 | | |
| Gracilibacillus halophilus YIM- | N4WKF3 | | | N4WBA0 | | |
| C55.5 | | | | | | |
| Gramella forsetii (strain | | | | A0LYA7; | | |
| KT0803) | | | | A0LZ76 | | |
| Granulicella mallensis (strain | | | | G8NQI9; | | |
| ATCC BAA-1857/DSM 23137/ | | | | G8NRG9; | | |
| MP5ACTX8) | | | | G8NYI7 | | |
| Grosmannia clavigera (strain | | F0XC21 | | F0X7P7; | | |
| kw1407/UAMH 11150) (Blue | | | | F0XCC7; | | |
| stain fungus) (Graphiocladiella | | | | F0XL68 | | |
| Clavigera) | | | | | | |
| Haliscomenobacter hydrossis | F4KPM4; | | | F4KZA8; | | |
| (strain ATCC 27775/DSM 1100/ | F4KXA7; | | | F4L775 | | |
| LMG 10767/O) | F4L5U2; | | | | | |
| | F4L8A5; | | | | | |
| | F4L8A6 | | | | | |
| Haloferax alexandrinus JCM | | | | M0ID98 | | |
| 10717 | | | | | | |
| Haloferax gibbonsii ATCC 33959 | | | | M0HP18 | | |
| Haloferax prahovense DSM | | | | M0FUA1; | | |
| 18310 | | | | M0FWA8 | | |
| Haloferax sp. BAB2207 | | | | L5NVS7 | | |
| Halogranum salariumB-1 | | | | J2Z9V7 | | |
| Halomonas boliviensis LC1 | G9EHD3 | | | | | |
| Halomonas sp. HAL1 | G4F1W1 | | | | | |
| Halopiger xanaduensis (strain | F8DCC2 | | | | | |
| DSM 18323/JCM 14033/SH- | | | | | | |
| 6) | | | | | | |
| Haloplasma contractile SSD- | | | | F7Q1V1 | | |
| 17B | | | | | | |
| Halorhabdus tiamatea SARL4B | F7PJ22; | | | F7PJI1; | | |
| | F7PJ23; | | | F7PK87; | | |
| | F7PQV5; | | | F7PM09 | | |
| | F7PQV6 | | | | | |
| Halorhabdus utahensis (strain | C7NV87 | | | C7NMF0; | | |
| DSM 12940/JCM 11049/AX- | | | | C7NMH6; | | |
| 2) | | | | C7NNQ1; | | |
| | | | | C7NQD4 | | |
| Halosimplex carlsbadense 2-9-1 | M0CLR3; | | | M0CAN2; | | |
| | M0CNI7; | | | M0CUR7 | | |
| | M0CP61; | | | | | |
| | M0CQM5 | | | | | |
| Haloterrigena salina JCM 13891 | | | | M0BWT1; | | |
| | | | | M0BYH9 | | |
| Haloterrigena turkmenica | D2RTV2 | | | D2S1R0; | | |
| (strain ATCC 51198/DSM 5511/ | | | | D2S1R8 | | |
| NCIMB 13204/VKM B-1734) | | | | | | |
| (Halococcus turkmenicus) | | | | | | |
| Halothermothrix orenii (strain | | | B8D1V0 | B8CZV1 | | |
| H 168/OCM 544/DSM 9562) | | | | | | |
| Herpetosiphon aurantiacus | A9B286 | | | A9AZL2 | A9B7H2 | |
| (strain ATCC 23779/DSM 785) | | | | | | |
| Hirschia baltica (strain ATCC | C6XQH5; | | | C6XQH8 | | |
| 49814/DSM 5838/IFAM | C6XRN4 | | | | | |
| 1418) | | | | | | |
| Holomastigotoides mirabile | | C0STU7; | | | | |
| | | C0STU9; | | | | |
| | | C0STV1 | | | | |
| Humicola grisea | P79046 | | | | | |
| Humicola grisea var. | | Q9HGE1 | | | | |
| thermoidea | | | | | | |
| Humicola insolens (Soft-rot | M4MEY9; | P55334 | | | | |
| fungus) | M4MGK7; | | | | | |
| | M4MLB5 | | | | | |
| Hyaloperonospora | M4BCI2; | | | | | |
| arabidopsidis (strain Emoy2) | M4C1Z6 | | | | | |
| (Downy mildew agent) | | | | | | |
| (Peronospora arabidopsidis) | | | | | | |
| Hypocrea atroviridis (strain | G9NXF5 | G9NE77; | G9N150; | G9NS03; | | G9NQN0; |
| ATCC 20476/IMI 206040) | | G9NQ12; | G9NRI8 | G9NZD6; | | G9P0X1 |
| (Trichoderma atroviride) | | G9NRZ0; | | G9P412; | | |
| | | G9PC46 | | G9P8J0; | | |
| | | | | G9PBA1 | | |
| Hypocrea jecorina (strain | G0RA32 | G0R947; | G0RE86; | G0RIU2 | | G0RXL3 |
| QM6a) (Trichoderma reesei) | | G0RUP7; | G0RVQ8 | | | |
| | | G0RWY3 | | | | |
| Hypocrea jecorina | Q9P973 | B2CNY5; | | | | |
| (Trichoderma reesei) | | B2CZF9; | | | | |
| | | P36217; | | | | |
| | | P36218; | | | | |
| | | Q02244; | | | | |
| | | Q99015; | | | | |
| | | Q9HGT9 | | | | |
| Hypocrea orientalis | | H9C5T6; | | | | |
| | | H9C5T7 | | | | |
| Hypocrea rufa (Trichoderma | | A0T2F0; | | | | |
| viride) | | Q7Z8Q3; | | | | |
| | | Q9UVF9 | | | | |
| Hypocrea virens (strain Gv29-8/ | G9MUR3; | G9MJY8; | G9N047; | G9MJ74; | | G9MX26 |
| FGSC 10586) (Gliocladium | G9NBD2 | G9MV13; | G9N118 | G9MNG4; | | |
| virens) (Trichoderma virens) | | G9MX24; | | G9N056 | | |
| | | G9N9X8 | | | | |
| Indibacter alkaliphilus LW1 | | | | S2DLH8 | | |
| Isoptericola variabilis (strain | F6FTN6; | | | F6FRE2; | | |
| 225) | F6FTN6; | | | F6FX81; | | |
| | F6FUN1 | | | F6FX86 | | |
| Isosphaera pallida (strain ATCC | E8R166 | | | | | |
| 43644/DSM 9630/IS1B) | | | | | | |
| Janthinobacterium sp. HH01 | | | L9PKD3 | L9PDB4 | | |
| Jeongeupia naejangsanensis | | | | | E2G4E3 | |
| Jonesia denitrificans (strain | C7R1S8; | C7R2M6 | | C7R0B5; | | |
| ATCC 14870/DSM 20603/CIP | C7R1S9; | | | C7R0C1; | | |
| 55134) (Listeria denitrificans) | C7R4R8; | | | C7R5J7; | | |
| | C7R5M3 | | | C7R5J8 | | |
| Joostella marina DSM 19592 | | | | I3C7P2 | | |
| Kineococcus radiotolerans | A6W5F0; | | | A6W430; | | |
| (strain ATCC BAA-149/DSM | A6W6W7 | | | A6WB18 | | |
| 14245/SRS30216) | | | | | | |
| Kitasatospora setae (strain | E4N6Z2; | E4N0N4 | | | | |
| ATCC 33774/DSM 43861/ | E4NJK1; | | | | | |
| JCM 3304/KCC A-0304/NBRC | E4NJK3 | | | | | |
| 14216/KM-6054) | | | | | | |
| (Streptomyces setae) | | | | | | |
| Klebsiella pneumoniae | | | | S2AN29; | S2BWB6 | |
| 361_1301 | | | | S2BAK9 | | |
| Klebsiella pneumoniae | | | | S2BUB8; | S2CPT0 | |
| 440_1540 | | | | S2CK54 | | |
| Klebsiella pneumoniae | | | | S2CCK3; | S2CGV6 | |
| 500_1420 | | | | S2CPU9 | | |
| Klebsiella pneumoniae | | | | S2D2I0; | S2E9V6 | |
| 540_1460 | | | | S2DFD3 | | |
| Klebsiella pneumoniae | | | | S2D619; | S2EDY4 | |
| 646_1568 | | | | S2E6U7 | | |
| Klebsiella pneumoniae | | | | S2H0B1; | | |
| DMC0526 | | | | S2HKI9 | | |
| Klebsiella pneumoniae KP-11 | | | | S2B5K5; | S2BLT0 | |
| | | | | S2C1Y9; | | |
| | | | | S2C9L1 | | |
| Klebsiella pneumoniae KP-7 | | | | S1SQ35; | S1SV73 | |
| | | | | S1TIW6; | | |
| | | | | S1TKF6 | | |
| Klebsiella pneumoniae UHKPC | | | | S2G248; | S2GBI0 | |
| 52 | | | | S2GIQ0 | | |
| Klebsiella pneumoniae | | | | S1UF32; | S1UAK9 | |
| UHKPC01 | | | | S1UHK6 | | |
| Klebsiella pneumoniae | | | | S1WME2; | S1XTJ9 | |
| UHKPC04 | | | | S1XC84 | | |
| Klebsiella pneumoniae | | | | S2FKF8; | S2G6D5 | |
| UHKPC05 | | | | S2H881 | | |
| Klebsiella pneumoniae | | | | S1TL14; | S1UA15 | |
| UHKPC09 | | | | S1VCV2 | | |
| Klebsiella pneumoniae | | | | S1X0S3; | S1X716 | |
| UHKPC22 | | | | S1XMN7 | | |
| Klebsiella pneumoniae | | | | R9BLI5; | R9BRD4 | |
| UHKPC23 | | | | R9BXA8 | | |
| Klebsiella pneumoniae | | | | S1VJG0; | S1VBG3 | |
| UHKPC24 | | | | S1WCQ1 | | |
| Klebsiella pneumoniae | | | | S1VNB0; | S1W2E4 | |
| UHKPC26 | | | | S1VPB0 | | |
| Klebsiella pneumoniae | | | | S1VT44; | S1WWS5 | |
| UHKPC27 | | | | S1WQ23 | | |
| Klebsiella pneumoniae | | | | S2HEN9; | S2I3E5 | |
| UHKPC29 | | | | S2IAH9 | | |
| Klebsiella pneumoniae | | | | S2ISJ5; | S2JBK9 | |
| UHKPC32 | | | | S2J4Q2 | | |
| Klebsiella pneumoniae | | | | S1TGU4; | S1T657 | |
| UHKPC40 | | | | S1TXD7 | | |
| Klebsiella pneumoniae | | | | S2FLY4; | S2H6D0 | |
| UHKPC45 | | | | S2FTA1 | | |
| Klebsiella pneumoniae | | | | S2IIL4; | S2IXU6 | |
| UHKPC48 | | | | S2IMK5 | | |
| Klebsiella pneumoniae | | | | S2EG50; | S2F816 | S2EXX9 |
| UHKPC57 | | | | S2EKP4 | | |
| Klebsiella pneumoniae | | | | S1UDV3; | S1V0M2 | |
| UHKPC81 | | | | S1UE26 | | |
| Klebsiella pneumoniae | | | | S1XD98; | S1XDD1 | |
| VAKPC252 | | | | S1XHC5 | | |
| Klebsiella pneumoniae | | | | S1Y650; | S1XML5 | |
| VAKPC254 | | | | S1YCL0 | | |
| Klebsiella pneumoniae | | | | S1YMN0; | S1Z892 | |
| VAKPC269 | | | | S1YPR4 | | |
| Klebsiella pneumoniae | | | | S1Z5D5; | S1ZL93 | |
| VAKPC270 | | | | S1ZGA0 | | |
| Klebsiella pneumoniae | | | | S2A8Y2; | S1ZYL5 | |
| VAKPC276 | | | | S2AJI6 | | |
| Klebsiella pneumoniae | | | | S2GVS3; | S2HET2 | |
| VAKPC278 | | | | S2H9S4 | | |
| Klebsiella pneumoniae | | | | S1Z008; | S1ZYJ4 | |
| VAKPC280 | | | | S1Z4Q5 | | |
| Klebsiella pneumoniae | | | | S2A8I7; | S2AFK2 | |
| VAKPC297 | | | | S2AV55 | | |
| Klebsiella pneumoniae | | | | S2AFV4; | S2AG24 | |
| VAKPC309 | | | | S2BBV1 | | |
| Kocuria sp. MN22 | B8XY24 | | | | | |
| Kribbella flavida (strain DSM | | | | D2PQJ1; | | D2PTT1; |
| 17836/JCM 10339/NBRC | | | | D2PQJ2 | | D2PTT3 |
| 14399) | | | | | | |
| Ktedonobacter racemifer DSM | D6TBL5; | | D6U4P3 | D6TQB1; | | |
| 44963 | D6TTB3; | | | D6TQZ9; | | |
| | D6TTB3 | | | D6TU44; | | |
| | | | | D6U0C1 | | |
| Laccaria bicolor (strain S238N- | | | B0D052; | | | |
| H82/ATCC MYA-4686) | | | B0D053; | | | |
| (Bicoloured deceiver) (Laccaria | | | B0D7U4; | | | |
| laccata var. bicolor) | | | B0DIW4; | | | |
| | | | B0DUW6; | | | |
| | | | B0E263 | | | |
| Lachnospiraceae bacterium | F7KCR6 | | | | | |
| 3_1_57FAA_CT1 | | | | | | |
| Lactobacillus gigeriorum CRBIP | | | | | I7J3F3; | |
| 24.85 | | | | | I7K0A5 | |
| Lactobacillus paracasei subsp. | | | S2SDL4 | | | |
| paracasei Lpp126 | | | | | | |
| Lactobacillus pasteurii CRBIP | | | | | I7LES7 | |
| 24.76 | | | | | | |
| Lactobacillus pentosus IG1 | | | | G0M4L2 | | |
| Lactobacillus pentosus KCA1 | | | | I9KYJ8 | | |
| Lactobacillus reuteri (strain | | | | | A5VLT0 | |
| DSM 20016) | | | | | | |
| Lactobacillus reuteri 100-23 | | | | | B3XPX3 | |
| Lactobacillus rhamnosus (strain | | | | C7TN46 | | |
| Lc 705) | | | | | | |
| Lactobacillus rhamnosus ATCC | | | G7V0V4 | | | |
| 8530 | | | | | | |
| Lactococcus lactis subsp. lactis | | | | | A9QSM5 | |
| (strain KF147) | | | | | | |
| Leadbetterella byssophila | E4RQT2; | | | E4RQV9; | | |
| (strain DSM 17132/KACC | E4RUD3; | | | E4RSC8; | | |
| 11308/4M15) | E4RWD4 | | | 4RSQ5; | | |
| | | | | E4RWC8; | | |
| | | | | E4RWF2; | | |
| | | | | E4RY23; | | |
| | | | | E4RYF2 | | |
| Lechevalieria sp. HJ3 | M4GR23 | | | | | |
| Leeuwenhoekiella blandensis | | | | A3XLS2 | | |
| (strain CECT 7118/CCUG | | | | | | |
| 51940/MED217) | | | | | | |
| (Flavobacterium sp. (strain | | | | | | |
| MED217)) | | | | | | |
| Lentinula edodes (Shiitake | | | C5NN25 | | | |
| mushroom) (Lentinus edodes) | | | | | | |
| Lentisphaera araneosa | A6DME7; | | | | | |
| HTCC2155 | A6DPD2 | | | | | |
| Leptolyngbya sp. PCC 7375 | K9FG18 | | | | | |
| Leptosphaeria maculans (strain | E4ZH02; | E4ZRR9; | | E4ZNM6 | | |
| JN3/isolate v23.1.3/race Av1- | E5A1T3; | E5A0Q4 | | | | |
| 4-5-6-7-8) (Blackleg fungus) | E5AEE4 | | | | | |
| (Phoma lingam) | | | | | | |
| Leptospira kirschneri serovar | | | | S3UC27 | | |
| Cynopteri str. 3522 CT | | | | | | |
| Leptospira wolbachii serovar | | | | R9A4Z6 | | |
| Codice str. CDC | | | | | | |
| Leptospira yanagawae serovar | | | | R8ZTE7 | | |
| Saopaulo str. Sao Paulo = ATCC | | | | | | |
| 700523 | | | | | | |
| Leucoagaricus gongylophorus | | A6YAP7 | | | | |
| (Leaf-cutting ant fungus) | | | | | | |
| Macrophomina phaseolina | K2QV81; | K2RN85 | | K2R7I9; | | K2S0D7; |
| (strain MS6) (Charcoal rot | K2RQP8; | | | K2RF14; | | K2SL91 |
| fungus) | K2RU22; | | | K2RHU9; | | |
| | K2RX09; | | | K2RJA1; | | |
| | K2SBN0; | | | K2RL04; | | |
| | K2SN80 | | | K2RMA7; | | |
| | | | | K2RTE6; | | |
| | | | | K2RVK0; | | |
| | | | | K2RX85; | | |
| | | | | K2RXD7; | | |
| | | | | K2S1B5; | | |
| | | | | K2S2A2; | | |
| | | | | K2S2B1; | | |
| | | | | K2S9V7; | | |
| | | | | K2SC12; | | |
| | | | | K2SDF9; | | |
| | | | | K2SLY5; | | |
| | | | | K2SPE5; | | |
| | | | | K2SPP5; | | |
| | | | | K2SSF0 | | |
| Magnaporthe grisea | Q01176; | Q92244; | | | | |
| (Crabgrass-specific blast | Q8J1Y4; | Q92245 | | | | |
| fungus) (Pyricularia grisea) | Q8NJ73 | | | | | |
| Magnaporthe oryzae (strain 70- | G4MLU0; | G4MVY2; | | G4MQZ5 | | |
| 15/ATCC MYA-4617/FGSC | G4MPQ7; | G4MWS3; | | | | |
| 8958) (Rice blast fungus) | G4MTF8; | G4N696; | | | | |
| (Pyricularia oryzae) | G4N1Y8; | G4NA54; | | | | |
| | G4NBN8; | P55335 | | | | |
| | G4NIM7 | | | | | |
| Magnaporthe oryzae (strain | L7IQU4; | L7J633; | | L7JDX3 | | |
| P131) (Rice blast fungus) | L7J0I5; | L7JAW6; | | | | |
| (Pyricularia oryzae) | L7J7U3; | L7JKE7; | | | | |
| | L7JBZ1; | L7JPY5; | | | | |
| | L7JKU2; | L7JRJ2 | | | | |
| | L7JMZ0 | | | | | |
| Magnaporthe oryzae (strain | L7HNG2; | L7HXF3; | | L7HZQ6 | | |
| Y34) (Rice blast fungus) | L7HV75; | L7I7Y0; | | | | |
| (Pyricularia oryzae) | L7HWI0; | L7I9I6; | | | | |
| | L7I1P2; | L7IGE5; | | | | |
| | L7I4J9; | L7IJQ4 | | | | |
| | L7IJX5 | | | | | |
| Magnaporthe poae (strain | M4FX28; | M4FWQ4; | | M4G7H5 | | |
| ATCC 64411/73-15) (Kentucky | M4G7X9; | M4GA15 | | | | |
| bluegrass fungus) | M4G9A5; | | | | | |
| | M4G9B8; | | | | | |
| | M4G9K2; | | | | | |
| | M4GFG0 | | | | | |
| Mahella australiensis (strain | F3ZYT6; | | | F3ZWG9 | F3ZWI4 | F3ZY55 |
| DSM 15567/CIP 107919/50-1 | F4A379 | | | | | |
| BON) | | | | | | |
| Manganese-oxidizing | Q1YH83 | | | | | |
| bacterium (strain SI85-9A1) | | | | | | |
| Mariniradius saccharolyticus | M7XVQ1 | | | | | |
| AK6 | | | | | | |
| Marssonina brunnea f. sp. | K1WXU3; | K1WWU0 | | K1WVY7 | | |
| multigermtubi (strain MB_m1) | K1WY01; | | | | | |
| (Marssonina leaf spot fungus) | K1WYP4 | | | | | |
| Massilia timonae CCUG 45783 | K9DCN2 | | | K9DQJ9 | | |
| Medicago truncatula (Barrel | G7J8H6; | | | | | |
| medic) (Medicago tribuloides) | G7KWV0 | | | | | |
| Meiothermus ruber (strain | D3PLV4; | | | | | |
| ATCC 35948/DSM 1279/VKM | M9X5U0 | | | | | |
| B-1258/21) (Thermus ruber) | | | | | | |
| Melampsora larici-populina | F4RD01; | | | F4R743; | | |
| (strain 98AG31/pathotype 3- | F4RYZ6; | | | F4RQX7; | | |
| 4-7) (Poplar leaf rust fungus) | F4S1S2; | | | F4S209 | | |
| | F4S1T6; | | | | | |
| | F4SE02 | | | | | |
| Melioribacter roseus (strain | I6Z9A7; | | I6YUI2 | I7A267 | | |
| P3M) | I7A603 | | | | | |
| Mesotoga prima MesG1.Ag.4.2 | I2F7G0 | | | | | |
| Mesotoga sp. PhosAc3 | N1JM60 | | | | | |
| Methanospirillum hungatei JF-1 | | | | | | Q2FMM6 |
| (strain ATCC 27890/DSM 864/ | | | | | | |
| NBRC 100397/JF-1) | | | | | | |
| Methylobacterium extorquens | | | | | H1KUI5 | |
| DSM 13060 | | | | | | |
| Methylobacterium | M7XWQ4 | | | | | |
| mesophilicum SR1.6/6 | | | | | | |
| Methylobacterium nodulans | | | | | B8IRA6 | |
| (strain ORS2060/LMG 21967) | | | | | | |
| Methylobacterium | B1LZ39 | | | | | |
| radiotolerans (strain ATCC | | | | | | |
| 27329/DSM 1819/JCM 2831) | | | | | | |
| Methylobacterium sp. GXF4 | I9CR70 | | | | | |
| Micavibrio aeruginosavorus | | | G2KNR9 | | | |
| (strain ARL-13) | | | | | | |
| Microbacterium | | | | H8E8R0 | | |
| laevaniformans OR221 | | | | | | |
| Microbispora corallina | E2IHD5; | | | | | |
| | E2IHD8 | | | | | |
| Microbulbifer hydrolyticus | | Q693B5 | | | | |
| Microcoleus sp. PCC 7113 | K9WAK5 | | | | | |
| Microcoleus vaginatus FGP-2 | | | | | | F5UEX4 |
| Micromonospora aurantiaca | D9SZ35; | D9SZ92 | | D9T229; | | |
| (strain ATCC 27029/DSM | D9SZ74; | | | D9TES0 | | |
| 43813/JCM 10878/NBRC | D9SZU6; | | | | | |
| 16125/INA 9442) | D9T5J5; | | | | | |
| | D9T5J8 | | | | | |
| Micromonospora lupini str. | I0KZ65; | I0L6W9 | | I0L4S8 | | |
| Lupac 08 | I0L2K8; | | | | | |
| | I0L3Z2; | | | | | |
| | I0L712; | | | | | |
| | I0L7C9 | | | | | |
| Micromonospora sp. (strain L5) | E8S118; | E8S053 | | E8RXE6; | | |
| | E8S157; | | | E8S4S8; | | |
| | E8S646; | | | E8SCA5; | | |
| | E8S6C2; | | | E8SCC3 | | |
| | E8SCW9 | | | | | |
| Micromonospora sp. ATCC | C4RB10; | C4RFE5; | | | | |
| 39149 | C4RG47; | C4RJH9; | | | | |
| | C4RGY4; | C4RQR4 | | | | |
| | C4RGZ5; | | | | | |
| | C4RH32; | | | | | |
| | C4RL73; | | | | | |
| | C4RMC7; | | | | | |
| | C4RN65; | | | | | |
| | C4RND6 | | | | | |
| Modestobacter marinus (strain | | | | I4ERV6 | | |
| BC501) | | | | | | |
| Moniliophthora perniciosa | E2LBK4; | E2LFE5; | | | | |
| (strain FA553/isolate CP02) | E2LK99; | E2LYQ6 | | | | |
| (Witches'-broom disease | E2LPD5; | | | | | |
| fungus) (Marasmius | E2LR18 | | | | | |
| perniciosus) | | | | | | |
| Monosiga brevicollis | A9UZL2 | | | | | |
| (Choanoflagellate) | | | | | | |
| Moorea producens 3L | F4XKE2 | | | | | |
| Morchella spongiola | | I6LKU3 | | | | |
| Mucilaginibacter paludis DSM | H1YFS9; | H1YIW2 | H1Y870; | H1XZF3; | H1Y041; | H1YA93; |
| 18603 | H1YHR8 | | H1YFM1 | H1Y274; | H1YFS5; | H1YCA1; |
| | | | | H1Y349; | H1YH20 | H1YHR9 |
| | | | | H1Y350; | | |
| | | | | H1Y754; | | |
| | | | | H1Y8J3; | | |
| | | | | H1Y8J5; | | |
| | | | | H1YA21; | | |
| | | | | H1YBP9; | | |
| | | | | H1YF53; | | |
| | | | | H1YFI1; | | |
| | | | | H1YFR6; | | |
| | | | | H1YFS6; | | |
| | | | | H1YFS7; | | |
| | | | | H1YFU0; | | |
| | | | | H1YHR4; | | |
| | | | | H1YIH8 | | |
| Muricauda ruestringensis | | | | G2PQW9; | | |
| (strain DSM 13258/LMG | | | | G2PSI0 | | |
| 19739/B1) | | | | | | |
| Mycobacterium vanbaalenii | | | | | A1TEN4 | |
| (strain DSM 7251/PYR-1) | | | | | | |
| Mycosphaerella fijiensis (strain | M3A7S3 | | M2Z992 | M2YL47; | | |
| CIRAD86) (Black leaf streak | | | | M2YVV5; | | |
| disease fungus) | | | | M2Z4V9; | | |
| (Pseudocercospora fijiensis) | | | | M2Z7N3; | | |
| | | | | M2ZFB0; | | |
| | | | | M3A3D6; | | |
| | | | | M3AL84; | | |
| | | | | M3AM13; | | |
| | | | | M3ARY1; | | |
| | | | | M3AX19; | | |
| | | | | M3B1N6; | | |
| | | | | M3B8I4; | | |
| | | | | N1Q7I8; | | |
| | | | | N1Q9Z8; | | |
| | | | | N1QC39 | | |
| Mycosphaerella graminicola | F9XFH3; | F9XDM7 | | F9XHT6 | | |
| (strain CBS 115943/IPO323) | F9XFH4 | | | | | |
| (Speckled leaf blotch fungus) | | | | | | |
| (Septoria tritici) | | | | | | |
| Mycosphaerella pini (strain | M2YHS3 | N1PCA4; | N1PRV3; | M2XLC4; | | |
| NZE10/CBS 128990) (Red band | | N1PGQ5 | N1Q2X9 | N1PCU1; | | |
| needle blight fungus) | | | | N1PD14; | | |
| (Dothistroma septosporum) | | | | N1PDN7; | | |
| | | | | N1PFB1; | | |
| | | | | N1PHW5; | | |
| | | | | N1PK69; | | |
| | | | | N1PU27; | | |
| | | | | N1Q185; | | |
| | | | | N1Q279 | | |
| Mycosphaerella populorum | | M3CYK1 | M3AXU9; | M3B2J4; | | |
| (strain SO2202) (Poplar stem | | | M3C0V7 | M3B383; | | |
| canker fungus) (Septoria | | | | M3BZL7; | | |
| musiva) | | | | M3C3U9; | | |
| | | | | M3CYP0; | | |
| | | | | N1QDC2; | | |
| | | | | N1QEG7; | | |
| | | | | N1QH05 | | |
| Nannochloropsis gaditana | | | | | I2CQP6; | |
| CCMP526 | | | | | K8YR29 | |
| Natrialba aegyptia DSM 13077 | | | | M0AGJ5; | | |
| | | | | M0AJV6; | | |
| | | | | M0AS18 | | |
| Natrialba asiatica (strain ATCC | | | | M0B599; | | |
| 700177/DSM 12278/JCM | | | | M0B6E4 | | |
| 9576/FERM P-10747/NBRC | | | | | | |
| 102637/172P1) | | | | | | |
| Natrialba taiwanensis DSM | | | | M0ACX9 | | |
| 12281 | | | | | | |
| Nectria haematococca (strain | C7Z894; | C7YSL3; | | C7YNH0; | | |
| 77-13-4/ATCC MYA-4622/ | C7ZH33; | C7ZN05 | | C7YVE8; | | |
| FGSC 9596/MPVI) (Fusarium | C7ZPB5 | | | C7Z0G3; | | |
| solani subsp. pisi) | | | | C7Z4G6; | | |
| | | | | C7ZEK9 | | |
| Neocallimastix frontalis (Rumen | | Q01421; | | | | |
| fungus) | | Q01426; | | | | |
| | | Q19N51; | | | | |
| | | Q19N52; | | | | |
| | | Q5YB84; | | | | |
| | | Q69IF9; | | | | |
| | | Q69IG0; | | | | |
| | | Q69IG1; | | | | |
| | | Q69IG2; | | | | |
| | | Q69IG3; | | | | |
| | | Q69IG4; | | | | |
| | | Q69IG9; | | | | |
| | | Q7Z8B8 | | | | |
| Neocallimastix patriciarum | Q02290 | B8YG19; | | | | |
| (Rumen fungus) | | P29127; | | | | |
| | | Q69IG5; | | | | |
| | | Q69IG6; | | | | |
| | | Q69IG7; | | | | |
| | | Q69IG8 | | | | |
| Neocallimastix sp. GMLF1 | | B5B3U7; | | | | |
| | | B8YQ34 | | | | |
| Neosartorya fischeri (strain | A1CX14; | A1DJ52; | | A1D133; | | |
| ATCC 1020/DSM 3700/FGSC | A1D5N3; | A1DJ68; | | A1D5W1; | | |
| A1164/NRRL 181) (Aspergillus | A1DNN0; | A1DN04; | | A1D7D9; | | |
| fischerianus) | A1DP82 | A1DNU5 | | A1DHW8; | | |
| | | | | A1DKY5 | | |
| Neosartorya fumigata | | E0X4B3 | | | | |
| (Aspergillus fumigatus) | | | | | | |
| Neosartorya fumigata (strain | Q0H904; | Q4WFZ8; | | Q4W930; | | |
| ATCC MYA-4609/Af293/CBS | Q4WLG5; | Q4WG11; | | Q4WR70; | | |
| 101355/FGSC A1100) | Q4WZ38 | Q4WLV2 | | Q4WYX7; | | |
| (Aspergillus fumigatus) | | | | Q4X0A5 | | |
| Neosartorya fumigata (strain | B0XM69; | B0XXD9; | | B0XPB0; | | |
| CEA10/CBS 144.89/FGSC | B0XZI7; | B0XXF3; | | B0XTS5; | | |
| A1163) (Aspergillus fumigatus) | B0Y6E0 | B0Y8Q8 | | B0XZW5; | | |
| | | | | B0YDT3 | | |
| Nesterenkonia xinjiangensis | | D1KJJ7 | | | | |
| Neurospora crassa | | Q6MVR8 | | | | |
| Neurospora crassa (strain ATCC | Q7RW51; | Q1K5S8; | Q7M4T0 | | | |
| 24698/74-OR23-1A/CBS | Q7S0Y0; | Q7SDQ1 | | | | |
| 708.71/DSM 1257/FGSC 987) | Q7S3P8; | | | | | |
| | Q7S6C2 | | | | | |
| Neurospora tetrasperma (strain | F8ML05; | F8MAI8; | | | | |
| FGSC 2508/ATCC MYA-4615/ | F8MVA4; | F8N4C2 | | | | |
| P0657) | F8MVE8; | | | | | |
| | F8MWJ7 | | | | | |
| Neurospora tetrasperma (strain | G4URG1; | G4UC47 | | | | |
| FGSC 2509/P0656) | G4UZW8; | | | | | |
| | G4V133; | | | | | |
| | G4V1J7 | | | | | |
| Niabella soli DSM 19437 | H1NJD4 | | | H1NKJ3; | | |
| | | | | H1NMW4; | | |
| | | | | H1NP08; | | |
| | | | | H1NP79; | | |
| | | | | H1NPW5; | | |
| | | | | H1NPW7 | | |
| Niastella koreensis (strain DSM | G8TBM0; | G8TR85 | | G8TBK1; | | |
| 17620/KACC 11465/GR20- | G8TLZ6; | | | G8TD73; | | |
| 10) | G8TN83; | | | G8TIU4; | | |
| | G8TR78 | | | G8TM60 | | |
| Nocardioidaceae bacterium | E9UYU8; | | | | | |
| Broad-1 | E9UZP1; | | | | | |
| | E9V1M6 | | | | | |
| Nocardioides sp. (strain BAA- | | | | A1SQC3 | | |
| 499/JS614) | | | | | | |
| Nocardiopsis alba (strain ATCC | J7L874 | | | | | |
| BAA-2165/BE74) | | | | | | |
| Nocardiopsis dassonvillei | D7AUR0; | D7AYW2 | | D7B0M6 | | |
| (strain ATCC 23218/DSM | D7AWS0; | | | | | |
| 43111/IMRU 509/JCM 7437/ | D7B7I8 | | | | | |
| NCTC 10488) (Actinomadura | | | | | | |
| dassonvillei) | | | | | | |
| Nostoc azollae (strain 0708) | | | | D7E2T1 | | |
| (Anabaena azollae (strain | | | | | | |
| 0708)) | | | | | | |
| Nostoc punctiforme (strain | B2IZC2; | | | B2J4N3 | | |
| ATCC 29133/PCC 73102) | B2IZQ1 | | | | | |
| Nostoc sp. (strain ATCC 29411/ | K9QN60 | | | | | |
| PCC 7524) | | | | | | |
| Nostoc sp. (strain PCC 7120/ | Q8YNW3 | | | | | |
| UTEX 2576) | | | | | | |
| Novosphingobium | | | | A4XEM1; | | |
| aromaticivorans (strain DSM | | | | Q2G474 | | |
| 12444) | | | | | | |
| Novosphingobium sp. AP12 | | | | J3AP83 | | |
| Odoribacter laneus YIT 12061 | | | | H1DFV6 | | |
| Odoribacter splanchnicus | | | | F9Z3P7 | | |
| (strain ATCC 29572/DSM | | | | | | |
| 20712/JCM 15291/NCTC | | | | | | |
| 10825/1651/6) (Bacteroides | | | | | | |
| splanchnicus) | | | | | | |
| Odoribacter splanchnicus | | | | R6FGR2 | | |
| CAG:14 | | | | | | |
| Oenococcus oeni ATCC BAA- | | | A0NKZ1 | | | |
| 1163 | | | | | | |
| Oligotropha carboxidovorans | B6JDZ9; | | | | | |
| (strain ATCC 49405/DSM 1227/ | F8BUX8 | | | | | |
| OM5) | | | | | | |
| Oligotropha carboxidovorans | F8BN97 | | | | | |
| (strain OM4) | | | | | | |
| Ophiostoma piceae UAMH | | S3CHZ1 | | S3CKA9 | | |
| 11346 | | | | | | |
| Opitutaceae bacterium TAV1 | I6AU60; | | | | | |
| | I6AX96; | | | | | |
| | I6B079 | | | | | |
| Opitutaceae bacterium TAV5 | H1ILU1; | | | | H1IP78 | H1IVZ8; |
| | H1IR77; | | | | | H1IWK1; |
| | H1IU10; | | | | | H1IXA8 |
| | H1IYU3; | | | | | |
| | H1IZX6; | | | | | |
| | H1J0N6; | | | | | |
| | H1J0N7; | | | | | |
| | H1J1U9; | | | | | |
| | H1J1V0 | | | | | |
| Opitutus terrae (strain DSM | B1ZN37; | B1ZMX2 | | B1ZN35; | B1ZP97; | B1ZRW8 |
| 11246/PB90-1) | B1ZNF5; | | | B1ZN43; | B1ZP98; | |
| | B1ZPQ7; | | | B1ZP73; | B1ZPU3 | |
| | B1ZXE4; | | | B1ZPA5; | | |
| | B1ZXI4; | | | B1ZPL7; | | |
| | B2A0C7 | | | B1ZQY2; | | |
| | | | | B1ZRZ9; | | |
| | | | | B1ZXJ8; | | |
| | | | | B1ZZA2; | | |
| | | | | B1ZZI6 | | |
| Orpinomyces sp. (strain PC-2) | | Q92257 | | | | |
| Orpinomyces sp. FCT 2 | | D1LGU1 | | | | |
| Orpinomyces sp. LT-3 | | G3FNU2 | | | | |
| Orpinomyces sp. OUS1 | Q5K098 | | | | | |
| Oscillatoria acuminata PCC | K9TC14 | | | | | |
| 6304 | | | | | | |
| Oscillatoria nigro-viridis PCC | | | | | | K9VH41 |
| 7112 | | | | | | |
| Paecilomyces aerugineus | G8ZAH1 | | | | | |
| Paecilomyces sp. J18 | | D1G4K3 | | | | |
| Paecilomyces variotii | | P81536 | | | | |
| Paenibacillus barcinonensis | C7C5G8; | | | | | |
| | O69230; | | | | | |
| | O69231 | | | | | |
| Paenibacillus campinasensis | | F8UMP6; | | | | |
| | | M4N7N5; | | | | |
| | | M4N7S8; | | | | |
| | | Q2I6W5 | | | | |
| Paenibacillus curdlanolyticus | B1A3N2; | D3GKE3 | | | | |
| | E3WF08; | | | | | |
| | I4DXK6 | | | | | |
| Paenibacillus curdlanolyticus | E0IAR5; | E0IAB8 | E0IAR3; | | E0IFC0 | |
| YK9 | E0IFB1 | | E0IBL5 | | | |
| Paenibacillus lactis 154 | G4H9M3; | | | G4H8I7; | | |
| | G4HGM6; | | | G4H919; | | |
| | G4HGM7; | | | G4HAA5; | | |
| | G4HNG5 | | | G4HAX0; | | |
| | | | | G4HGM5; | | |
| | | | | G4HHG3 | | |
| Paenibacillus macerans | | Q45VU8 | | | | |
| (Bacillus macerans) | | | | | | |
| Paenibacillus mucilaginosus | F8F6P2; | F8F611 | | F8F862; | | |
| (strain KNP414) | F8F7P4; | | | F8FGI3 | | |
| | F8FB71; | | | | | |
| | F8FBP6; | | | | | |
| | F8FDW6; | | | | | |
| | F8FJM8 | | | | | |
| Paenibacillus mucilaginosus | H6N934; | H6NMP9 | | H6NHM4; | | |
| 3016 | H6NAV8; | | | H6NM09; | | |
| | H6NCA2; | | | H6NPE7 | | |
| | H6NJX8; | | | | | |
| | H6NQ08 | | | | | |
| Paenibacillus mucilaginosus | I0BC40; | I0BJW4 | | I0BHF1 | | |
| K02 | I0BDM2; | | | | | |
| | I0BKJ3; | | | | | |
| | I0BL51; | | | | | |
| | I0BLA7; | | | | | |
| | I0BMC3 | | | | | |
| Paenibacillus polymyxa | | E1AHZ6; | | P45796 | | |
| (Bacillus polymyxa) | | Q45VU9 | | | | |
| Paenibacillus polymyxa (strain | E0RDU1; | E0RJH8 | | E0RHQ6 | | |
| E681) | E0RKZ7; | | | | | |
| | E0RMV8 | | | | | |
| Paenibacillus polymyxa (strain | E3EB21 | E3EBI0 | E3EDI0 | E3EC02; | | |
| SC2) (Bacillus polymyxa) | | | | E3ECI5; | | |
| | | | | E3ED00; | | |
| | | | | E3EIR4; | | |
| | | | | E3EIR5 | | |
| Paenibacillus polymyxa M1 | I7KDI1; | I7JSJ6 | | G0VTT8 | | |
| | I7L4N8 | | | | | |
| Paenibacillus sp. (strain JDR-2) | A9QDS0; | C6D8U8 | C6D3J4 | C6CSG3; | C6CVZ5 | |
| | C6CRV0; | | | C6CXD7; | | |
| | C6D767; | | | C6CZH1; | | |
| | C6D776; | | | C6D076; | | |
| | C6D781 | | | C6D0M6; | | |
| | | | | C6D6C5; | | |
| | | | | C6D725; | | |
| | | | | C6D782 | | |
| Paenibacillus sp. Aloe-11 | H6CRI4; | H6CFA6 | | H6CEL3; | | |
| | H6CRY6 | | | H6CH30 | | |
| Paenibacillus sp. DG-22 | | A4GG22 | | | | |
| Paenibacillus sp. E18 | D6BQP4 | | | | | |
| Paenibacillus sp. enrichment | H9M7J2 | | | | | |
| culture clone 12-11 | | | | | | |
| Paenibacillus sp. HGF5 | F3M6U1; | F3MAL6 | | | | |
| | F3MB66 | | | | | |
| Paenibacillus sp. HPL-001 | B6VF01 | | | | | |
| Paenibacillus sp. HPL-002 | D5LRR5 | | | | | |
| Paenibacillus sp. HY8 | | A3QRI7 | | | | |
| Paenibacillus sp. ICGEB2008 | | G0YA74 | | | | |
| Paenibacillus sp. KCTC8848P | Q9F9B8 | Q9F9B9 | | | | |
| Paenibacillus sp. oral taxon 786 | C6IXI1; | C6J002 | | | | |
| str. D14 | C6J190 | | | | | |
| Paenibacillus sp. W-61 | Q8GHJ4 | Q1XGE6 | | | | |
| Paenibacillus terrae (strain HPL- | F1KBQ3; | G7VQ68; | | G7VPB2; | | |
| 003) | G7VTT7; | G7VWB2 | | G7VQ54; | | |
| | G7VZT2; | | | G7VZB2; | | |
| | G7W2I6 | | | G7W0C0 | | |
| Paenibacillus vortex V453 | E5YP28 | E5YXF6 | | E5YR32; | | |
| | | | | E5YR88; | | |
| | | | | E5Z0I4; | | |
| | | | | E5Z0I8; | | |
| | | | | E5Z0M8 | | |
| Paenibacillus xylaniclasticus | | I6ZTY5 | | | | |
| Paenibacillus xylanilyticus | G4WAA2 | | | | | |
| Paludibacter propionicigenes | E4T4X1; | | | E4T0W0; | E4T507 | |
| (strain DSM 17365/JCM 13257/ | E4T4Y6; | | | E4T2W7; | | |
| WB4) | E4T6U8 | | | E4T444; | | |
| | | | | E4T4X5; | | |
| | | | | E4T4X6; | | |
| | | | | E4T4X8; | | |
| | | | | E4T4Z9; | | |
| | | | | E4T501; | | |
| | | | | E4T503 | | |
| Pantoea ananatis (strain | F2ESH5 | | | | | |
| AJ13355) | | | | | | |
| Pantoea ananatis (strain LMG | D4GI13 | | | | | |
| 20103) | | | | | | |
| Pantoea ananatis LMG 5342 | G9APD8 | | | | | |
| Pantoea ananatis PA13 | G7UL32 | | | | | |
| Pantoea sp. (strain At-9b) | | | | | E6WHC0 | |
| Pantoea stewartii subsp. | H3RJD2 | | | | | |
| stewartii DC283 | | | | | | |
| Parabacteroides distasonis | | | A6LCW8; | A6LBN4; | | |
| (strain ATCC 8503/DSM 20701/ | | | A6LIF8 | A6LCT5; | | |
| NCTC 11152) | | | | A6LDZ6; | | |
| | | | | A6LEL1; | | |
| | | | | A6LGF7; | | |
| | | | | A6LGG1 | | |
| Parabacteroides merdae ATCC | | | | A7ABW3 | | |
| 43184 | | | | | | |
| Parabacteroides merdae | | | | K5ZPJ0 | | |
| CL09T00C40 | | | | | | |
| Parabacteroides sp. CAG:2 | | | R6IX10; | R6IKX4; | | |
| | | | R6JF29 | R6IMM2; | | |
| | | | | R6ISL1 | | |
| Parabacteroides sp. CAG:409 | | | R7J628 | | | |
| Paraprevotella clara YIT 11840 | G5SRV1 | | | G5SU69 | | |
| Paraprevotella xylaniphila YIT | F3QSV4 | | | F3QR01 | | |
| 11841 | | | | | | |
| Pectobacterium carotovorum | | | | C6D947 | | |
| subsp. carotovorum (strain | | | | | | |
| PC1) | | | | | | |
| Pectobacterium carotovorum | | | | J7L2K4 | | |
| subsp. carotovorum PCC21 | | | | | | |
| Pectobacterium wasabiae | | | | D0KID1; | | |
| (strain WPP163) | | | | D0KMJ4 | | |
| Pedobacter heparinus (strain | | | | C6XSM6; | | C6XSG7 |
| ATCC 13125/DSM 2366/NCIB | | | | C6XSN4; | | |
| 9290) | | | | C6XY23; | | |
| | | | | C6Y048; | | |
| | | | | C6Y0H0; | | |
| | | | | C6Y3T9 | | |
| Pedobacter saltans (strain ATCC | F0S5G3; | F0S4T0 | | F0S5E8; | | |
| 51119/DSM 12145/JCM | F0S6Y3 | | | F0S5F4; | | |
| 21818/LMG 10337/NBRC | | | | F0S6G7; | | |
| 100064/NCIMB 13643) | | | | F0SA37; | | |
| | | | | F0SA40; | | |
| | | | | F0SCQ5 | | |
| Pedosphaera parvula Ellin514 | B9XH31 | | | B9XBB3; | | B9XPN7 |
| | | | | B9XG29; | | |
| | | | | B9XH29; | | |
| | | | | B9XQQ1 | | |
| Penicillium canescens | C3VEV9; | C3VEV7; | | | | |
| | Q5S7A8 | C3VEV8; | | | | |
| | | C3VEW0 | | | | |
| Penicillium chrysogenum | B6F253; | B6F254 | | Q5H7M8; | | |
| (Penicillium notatum) | P29417; | | | Q75WE6 | | |
| | Q2PS23; | | | | | |
| | Q6PRW6 | | | | | |
| Penicillium chrysogenum | B6H9S6; | B6GYT7 | | B6GZA7; | | |
| (strain ATCC 28089/DSM 1075/ | B6HDC7; | | | B6GZL3; | | |
| Wisconsin 54-1255) | B6HPJ6 | | | B6H102; | | |
| (Penicillium notatum) | | | | B6H2Z7; | | |
| | | | | B6HDH5; | | |
| | | | | B6HE62 | | |
| Penicillium citrinum | B1B533 | Q2PGY1 | | | | |
| Penicillium decumbens | F1CHI3 | D3JYP8; | | | | |
| | | F1CHI4 | | | | |
| Penicillium digitatum (Green | J9WND0 | K4MMK3 | | | | |
| mold) | | | | | | |
| Penicillium digitatum (strain | K9FXX3 | K9GHZ3 | | K9FUA5 | | |
| Pd1/CECT 20795) (Green | | | | | | |
| mold) | | | | | | |
| Penicillium digitatum (strain | K9FFW7 | | | | | |
| PHI26/CECT 20796) (Green | | | | | | |
| mold) | | | | | | |
| Penicillium digitatum (strain | | | | K9G431 | | |
| PHI26/CECT 20796) (Green | | | | | | |
| mold) | | | | | | |
| Penicillium digitatum (strain | | K9GG34 | | | | |
| PHI26/CECT 20796) (Green | | | | | | |
| mold) | | | | | | |
| Penicillium funiculosum | Q5ZNB1 | Q9HFH0 | | | | |
| (Fruitlet core rot fungus) | | | | | | |
| Penicillium marneffei (strain | B6QN64 | B6QNW0; | | | | |
| ATCC 18224/CBS 334.59/QM | | B6QV47 | | | | |
| 7333) | | | | | | |
| Penicillium occitanis | | I3PW13 | | | | |
| Penicillium oxalicum | E1B2N4 | | | | | |
| Penicillium purpurogenum | Q9P8J1 | Q12666; | | | | |
| (Soft rot fungus) | | Q96W72 | | | | |
| Penicillium simplicissimum | P56588 | | | | | |
| Penicillium sp. 40 | | Q9UUQ2 | | | | |
| Penicillium sp. CGMCC 1669 | | D1GFE6 | | | | |
| Penicillium sp. enrichment | G9BY19 | | | | | |
| culture clone C1 | | | | | | |
| Penicillium sp. LYG 0704 | | E7DVW3 | | | | |
| Petrotoga mobilis (strain DSM | A9BJ30 | | | | | |
| 10674/SJ95) | | | | | | |
| Phaeodactylum tricornutum | B7FTY0 | | | | | |
| (strain CCAP 1055/1) | | | | | | |
| Phaeosphaeria nodorum | B6DQK5; | Q9UVY9 | | | | |
| (Glume blotch fungus) | B6DQK6; | | | | | |
| (Septoria nodorum) | B6DQK7; | | | | | |
| | B6DQK8 | | | | | |
| Phaeosphaeria nodorum (strain | Q0TXB3; | Q0TZE3; | | Q0U580; | | |
| SN15/ATCC MYA-4574/FGSC | Q0U923; | Q0U2J3; | | Q0UQC1 | | |
| 10173) (Glume blotch fungus) | Q0UA13; | Q0U5W9; | | | | |
| (Septoria nodorum) | Q0UBK2; | Q0UBJ9; | | | | |
| | Q0UMN4; | Q0UBV5; | | | | |
| | Q0UXC1; | Q0UF14 | | | | |
| | Q0V2I8 | | | | | |
| Phanerochaete carnosa (strain | K5VC42; | K5WVZ1 | K5UIX1; | K5VX22; | | |
| HHB-10118-sp) (White-rot | K5VZX9; | | K5W0K4; | K5W0R8; | | |
| fungus) (Peniophora carnosa) | K5WHC3; | | K5W192 | K5W9A5; | | |
| | K5WIK1; | | | K5WYD8 | | |
| | K5X6K8 | | | | | |
| Phanerochaete chrysosporium | B7SIW2; | | | I6XPK9 | | |
| (White-rot fungus) | G0ZCU2; | | | | | |
| (Sporotrichum pruinosum) | Q9HEZ1; | | | | | |
| | Q9HEZ2 | | | | | |
| Phenylobacterium zucineum | B4RAV8; | | | | | |
| (strain HLK1) | B4RGI4; | | | | | |
| | B4RGI6 | | | | | |
| Phialophora sp. CGMCC 3328 | F2VRY7 | | | | | |
| Photorhabdus asymbiotica | | | | | | C7BKA2 |
| subsp. asymbiotica (strain ATCC | | | | | | |
| 43949/3105-77) (Xenorhabdus | | | | | | |
| luminescens (strain 2)) | | | | | | |
| Phycisphaera mikurensis (strain | I0ICW6; | | | | | |
| NBRC 102666/KCTC 22515/ | I0ICW8; | | | | | |
| FYK2301M01) | I0ICW9 | | | | | |
| Phytophthora infestans (strain | D0N0W5; | | | | | |
| T30-4) (Potato late blight | D0NUP8; | | | | | |
| fungus) | D0NUP9 | | | | | |
| Phytophthora ramorum | H3GF46; | | | | | |
| (Sudden oak death agent) | H3GF56; | | | | | |
| | H3GZC7; | | | | | |
| | H3GZC9; | | | | | |
| | H3H2W4; | | | | | |
| | H3H4C0; | | | | | |
| | H3HAU6 | | | | | |
| Phytophthora sojae (strain | G4Z5Z9; | | G4ZEB0; | | | |
| P6497) (Soybean stem and root | G5A117; | | G4ZEB8; | | | |
| rot agent) (Phytophthora | G5A118; | | G4ZEC3; | | | |
| megasperma f. sp. glycines) | G5A8M8; | | G4ZEY4 | | | |
| | G5A8P6 | | | | | |
| Piriformospora indica (strain | G4TFF8; | G4TKT1; | | G4TQK0 | | |
| DSM 11827) | G4TFF9; | G4TKT3; | | | | |
| | G4TFG0; | G4TKT4; | | | | |
| | G4TFG1; | G4TKT5; | | | | |
| | G4TFG2; | G4TKV9; | | | | |
| | G4TFG3; | G4TNM5; | | | | |
| | G4TGH7; | G4TUA6; | | | | |
| | G4TIH8; | G4TWK7; | | | | |
| | G4TIH9; | G4U014; | | | | |
| | G4TM72; | G4U378; | | | | |
| | G4TM75; | G4U379 | | | | |
| | G4TM83; | | | | | |
| | G4TRC6; | | | | | |
| | G4TXD9; | | | | | |
| | G4TZC5 | | | | | |
| Piromyces communis | | B0FEV6; | | | | |
| | | Q9HFT3 | | | | |
| Piromyces sp. | | Q12667 | | | | |
| Piromyces sp. RRY-2002 | | Q49UB8 | | | | |
| Planctomyces brasiliensis | F0SMP4 | | | | | |
| (strain ATCC 49424/DSM 5305/ | | | | | | |
| JCM 21570/NBRC 103401/ | | | | | | |
| IFAM 1448) | | | | | | |
| Planctomyces limnophilus | D5SX37 | | | | | |
| (strain ATCC 43296/DSM 3776/ | | | | | | |
| IFAM 1008/290) | | | | | | |
| Plectosphaerella cucumerina | | Q38Q19 | | | | |
| Pleurocapsa sp. PCC 7327 | K9T0P3 | | | | | |
| Pleurotus ostreatus (Oyster | | B0FX60 | | | | |
| mushroom) (White-rot fungus) | | | | | | |
| Podospora anserina (strain S/ | B2ADU0; | B2A9A1; | | | | |
| ATCC MYA-4624/DSM 980/ | B2AFS1; | B2A9I4; | | | | |
| FGSC 10383) (Pleurage | B2AMK1; | B2AMH4; | | | | |
| anserina) | B2APG8; | B2B1K0; | | | | |
| | B2AQD3; | B2B3J5 | | | | |
| | B2AV20; | | | | | |
| | B2B5D0; | | | | | |
| | B2B789 | | | | | |
| Polyplastron multivesiculatum | B7FBK3; | O77398; | | | | |
| | Q9U0G1 | Q70WH8; | | | | |
| | | Q9XXV4 | | | | |
| Polysphondylium pallidum | | | D3BNM4 | | | |
| (Cellular slime mold) | | | | | | |
| Populus trichocarpa (Western | B9H179 | | | | | |
| balsam poplar) (Populus | | | | | | |
| balsamifera subsp. trichocarpa) | | | | | | |
| Postia placenta | D7REW5 | | | | | |
| Postia placenta (strain ATCC | B8P420; | | | | | |
| 44394/Madison 698-R) | B8P421; | | | | | |
| (Brown rot fungus) (Poria | B8PIA1; | | | | | |
| monticola) | B8PIA6 | | | | | |
| Prevotella bergensis DSM | D1PXP7; | | | D1PUA9 | | |
| 17361 | D1PXQ8 | | | | | |
| Prevotella bryantii | Q8GBY5 | | | | | |
| Prevotella bryantii B14 | D7SFG8; | | | D7SFG9; | | |
| | D8DUC2; | | | D7SFH2; | | |
| | D8DVU6 | | | D8DTH5; | | |
| | | | | D8DXZ6 | | |
| Prevotella buccae ATCC 33574 | E6K4F7; | | | E6K3N0 | | |
| | E6K4Q1 | | | | | |
| Prevotella buccae D17 | D3HX56; | | | D3HXU6 | | |
| | D3HXA9 | | | | | |
| Prevotella copri CAG:164 | | | | R6CPH1 | | |
| Prevotella copri DSM 18205 | D1PFS8 | | | D1PF41 | | |
| Prevotella dentalis (strain ATCC | F9D516; | | | F9D2E4; | | |
| 49559/DSM 3688/JCM 13448/ | L0JCN8 | | | F9D5H7 | | |
| NCTC 12043/ES 2772) | | | | | | |
| (Mitsuokella dentalis) | | | | | | |
| Prevotella denticola (strain | | | | F2KWT2 | | |
| F0289) | | | | | | |
| Prevotella denticola CRIS 18C-A | | | | F0H7D5 | | |
| Prevotella histicola F0411 | | | | G6AHW9 | | |
| Prevotella maculosa OT 289 | H1HKD2; | | | H1HM70 | | |
| | H1HKX2 | | | | | |
| Prevotella multisaccharivorax | | | | F8N7C8; | | F8N7G2 |
| DSM 17128 | | | | F8N7F6; | | |
| | | | | F8NA65; | | |
| | | | | F8NAI6; | | |
| | | | | F8NAI8; | | |
| | | | | F8NAJ6 | | |
| Prevotella oralis ATCC 33269 | | | | E7RN97 | | |
| Prevotella oris C735 | D7NDC5 | | | D7NEI4; | | |
| | | | | D7NF47 | | |
| Prevotella oris F0302 | | | | D1QN27; | | |
| | | | | D1QVE2 | | |
| Prevotella oulorum F0390 | | | | G1WDH1 | | |
| Prevotella ruminicola | P48789; | | | P48791; | | |
| (Bacteroides ruminicola) | P72234; | | | Q9WXE8 | | |
| | Q52307 | | | | | |
| Prevotella ruminicola (strain | D5ESF3; | | | D5EUP0; | | |
| ATCC 19189/JCM 8958/23) | D5EY13; | | | D5EXH7 | | |
| | D5EY24 | | | | | |
| Prevotella salivae DSM 15606 | | | | E6MRN8 | | |
| Prevotella sp. CAG:1124 | | | | R5KT10 | | |
| Prevotella sp. CAG:1185 | | | | R5MHM2; | | |
| | | | | R5MI29 | | |
| Prevotella sp. CAG:255 | | | | R5CZF5 | | |
| Prevotella sp. CAG:487 | | | | R5PFD1; | | |
| | | | | R5PG08; | | |
| | | | | R5PWQ9 | | |
| Prevotella sp. CAG:604 | | | | R6B4R2 | R6ANF3 | |
| Prevotella sp. CAG:732 | | | | R6XHL2 | | |
| Prevotella sp. CAG:924 | | | | R5F9F3; | | |
| | | | | R5FRR5 | | |
| Prevotella sp. MSX73 | J4TXG9; | | | J5HKH0 | | |
| | J5HRX3 | | | | | |
| Propionibacterium | | | | K7RSS0 | | |
| acidipropionici (strain ATCC | | | | | | |
| 4875/DSM 20272/JCM 6432/ | | | | | | |
| NBRC 12425/NCIMB 8070) | | | | | | |
| Propionibacterium avidum | | | | M9VFU4 | | |
| 44067 | | | | | | |
| Pseudallescheria sp. JSM-2 | | I6P974 | | | | |
| Pseudanabaena biceps PCC | L8MW36 | | | | | |
| 7429 | | | | | | |
| Pseudoalteromonas atlantica | Q15SG8 | | | | Q15WZ3 | |
| (strain T6c/ATCC BAA-1087) | | | | | | |
| Pseudoalteromonas sp. | G7F6N0; | | | | | |
| BSi20429 | G7F6N4; | | | | | |
| | G7F8Z8 | | | | | |
| Pseudoalteromonas sp. | | | | G7FX03 | | |
| BSi20495 | | | | | | |
| Pseudoalteromonas sp. | M5H0T7; | | | | | |
| Bsw20308 | M5H7A2; | | | | | |
| | M5H7K0 | | | | | |
| Pseudobutyrivibrio | | P83513; | | | | |
| xylanivorans | | Q704N9; | | | | |
| | | Q704P0 | | | | |
| Pseudomonas aeruginosa RP73 | | | | R9ZMU7 | | |
| Pseudomonas fluorescens | | | | | Q8RSY9 | |
| (strain SBW25) | | | | | | |
| Pseudomonas fluorescens | L7H6U6 | | | | | |
| BRIP34879 | | | | | | |
| Pseudomonas poae RE*1-1-14 | M4K4W2 | | | | | |
| Pseudomonas psychrotolerans | H0J717 | | | | | |
| L19 | | | | | | |
| Pseudomonas savastanoi pv. | D7I5N7 | | | | | |
| savastanoi NCPPB 3335 | | | | | | |
| Pseudomonas sp. ND137 | Q5KQS0 | Q8VUT4 | | | | |
| Pseudomonas sp. PE2 | Q84IG0 | | | | | |
| Pseudomonas syringae | L7FTA3 | | | | | |
| BRIP34876 | | | | | | |
| Pseudomonas syringae | L7G9Z4 | | | | | |
| BRIP34881 | | | | | | |
| Pseudomonas syringae | L7H854 | | | | | |
| BRIP39023 | | | | | | |
| Pseudomonas syringae Cit 7 | F3H260 | | | | | |
| Pseudomonas syringae pv. | F3JDD1 | | | | | |
| aceris str. M302273 | | | | | | |
| Pseudomonas syringae pv. | F3IX56 | | | | | |
| aptata str. DSM 50252 | | | | | | |
| Pseudomonas syringae pv. | K2S5Z7 | | | | | |
| avellanae str. ISPaVe013 | | | | | | |
| Pseudomonas syringae pv. | K2S751 | | | | | |
| avellanae str. ISPaVe037 | | | | | | |
| Pseudomonas syringae pv. | E7PAY8 | | | | | |
| glycinea str. B076 | | | | | | |
| Pseudomonas syringae pv. | E7PIN4 | | | | | |
| glycinea str. race 4 | | | | | | |
| Pseudomonas syringae pv. | F3FLN9 | | | | | |
| japonica str. M301072 | | | | | | |
| Pseudomonas syringae pv. | F3EJJ6 | | | | | |
| lachrymans str. M301315 | | | | | | |
| Pseudomonas syringae pv. mori | F3ES13 | | | | | |
| str. 301020 | | | | | | |
| Pseudomonas syringae pv. | F2ZD83 | | | | | |
| oryzae str. 1_6 | | | | | | |
| Pseudomonas syringae pv. | Q48D89 | | | | | |
| phaseolicola (strain 1448A/ | | | | | | |
| Race 6) | | | | | | |
| Pseudomonas syringae pv. pisi | F3G9X4 | | | | | |
| str. 1704B | | | | | | |
| Pseudomonas syringae pv. | Q4ZMT4 | | | | | |
| syringae (strain B728a) | | | | | | |
| Pseudomonas syringae pv. | L8N7F1 | | | | | |
| syringae B64 | | | | | | |
| Pseudomonas syringae pv. | F3K5T4 | | | | | |
| tabaci str. ATCC 11528 | | | | | | |
| Pseudoxanthomonas | E6WX38 | E6WTG4 | | E6WRK9; | E6WTI5 | |
| suwonensis (strain 11-1) | | | | E6WVC5 | | |
| Pseudozyma antarctica (strain | M9ME65; | M9LS78 | | | | |
| T-34) (Yeast) (Candida | M9MFL7 | | | | | |
| antarctica) | | | | | | |
| Psychrobacter sp. 2-17 | H6VBZ7 | | | | | |
| Puccinia graminis f. sp. tritici | E3KR71; | | | | | |
| (strain CRL 75-36-700-3/race | E3KR80; | | | | | |
| SCCL) (Black stem rust fungus) | E3KWH0; | | | | | |
| | E3L548 | | | | | |
| Puccinia triticina (isolate 1-1/ | J3PLV5; | | | | | |
| race 1 (BBBD)) (Brown leaf rust | J3PNK7; | | | | | |
| fungus) | J3Q1I0 | | | | | |
| Pyrenophora teres f. teres | E3RQI5; | E3RNK4; | | E3RH12; | | |
| (strain 0-1) (Barley net blotch | E3S3X7; | E3S3R6; | | E3RKG3 | | |
| fungus) (Drechslera teres f. teres) | E3S5R5; | E3S4Z8; | | | | |
| | E3S607 | E3S9S5 | | | | |
| Pyrenophora tritici-repentis | B2W0F8; | B2WG17; | | B2WI36 | | |
| (strain Pt-1C-BFP) (Wheat tan | B2W4V6; | B2WK18; | | | | |
| spot fungus) (Drechslera tritici- | B2WFS9; | B2WLG7 | | | | |
| repentis) | B2WHS1 | | | | | |
| Rahnella sp. (strain Y9602) | | | | | E8XTD0 | |
| Ramlibacter tataouinensis | | | | F5Y687 | F5XYQ3; | |
| (strain ATCC BAA-407/DSM | | | | | F5Y3B4 | |
| 14655/LMG 21543/TTB310) | | | | | | |
| Reinekea blandensis MED297 | | | | A4BFK6 | | |
| Rhizobium etli (strain CFN 42/ | Q2K5B0 | | | | | |
| ATCC 51251) | | | | | | |
| Rhizobium etli (strain CIAT 652) | B3PWG3 | | | | | |
| Rhizobium etli CNPAF512 | F2AD98 | | | | | |
| Rhizobium leguminosarum bv. | Q27SW6 | | | | | |
| trifolii | | | | | | |
| Rhizobium leguminosarum bv. | C6ATW9 | | | | | C6AY44 |
| trifolii (strain WSM1325) | | | | | | |
| Rhizobium leguminosarum bv. | B5ZZI1 | | | | | |
| trifolii (strain WSM2304) | | | | | | |
| Rhizobium leguminosarum bv. | J0C3G8 | | | | | |
| trifolii WSM2012 | | | | | | |
| Rhizobium leguminosarum bv. | J0W554 | | | | | |
| trifolii WSM2297 | | | | | | |
| Rhizobium leguminosarum bv. | I9NDD2 | | | | | |
| trifolii WSM597 | | | | | | |
| Rhizobium leguminosarum bv. | Q93L32 | | | | | |
| viciae | | | | | | |
| Rhizobium leguminosarum bv. | Q1MD47 | | | | | |
| viciae (strain 3841) | | | | | | |
| Rhizobium leguminosarum bv. | J0V352 | | | | | |
| viciae WSM1455 | | | | | | |
| Rhizobium lupini HPC(L) | K5DNP1 | | | | | |
| Rhizobium mesoamericanum | K0Q633 | | | | | |
| STM3625 | | | | | | |
| Rhizobium sp. CCGE 510 | J4TAV8 | | | | | |
| Rhizobium sp. CF122 | J2RAM9 | | | | | |
| Rhizobium sp. PDO1-076 | H4EZL9 | | | H4F2A3 | | |
| Rhizobium sp. Pop5 | K0W0K6 | | | | | |
| Rhizopus delemar (strain RA | | | | I1CUE3 | | |
| 99-880/ATCC MYA-4621/ | | | | | | |
| FGSC 9543/NRRL 43880) | | | | | | |
| (Mucormycosis agent) | | | | | | |
| (Rhizopus arrhizus var. | | | | | | |
| delemar) | | | | | | |
| Rhodanobacter fulvus Jip2 | I4VQD3 | I4VQH1 | | | | |
| Rhodanobacter sp. 115 | I4W9D9 | | | | | |
| Rhodobacter sp. SW2 | | | | | | C8S4A6 |
| Rhodoferax ferrireducens | | | Q21ZF6 | | | |
| (strain DSM 15236/ATCC BAA- | | | | | | |
| 621/T118) | | | | | | |
| Rhodomicrobium vannielii | E3I192 | | | | | |
| (strain ATCC 17100/ATH 3.1.1/ | | | | | | |
| DSM 162/LMG 4299) | | | | | | |
| Rhodopirellula baltica (strain | Q7UKV6 | | | | | |
| SH1) | | | | | | |
| Rhodopirellula baltica SH28 | K5C981; | | | K5DA16 | | |
| | K5CX22; | | | | | |
| | K5DBV6; | | | | | |
| | K5DKD9 | | | | | |
| Rhodopirellula baltica SWK14 | L7C9T0; | | | L7CJA9 | | |
| | L7CE85; | | | | | |
| | L7CJ24; | | | | | |
| | L7CN72 | | | | | |
| Rhodopirellula baltica WH47 | F2ALI4; | | | F2AZY1 | | |
| | F2AMF3; | | | | | |
| | F2ARX4; | | | | | |
| | F2B044 | | | | | |
| Rhodopirellula europaea 6C | M2A3W9; | | | M2A981 | | |
| | M2A832; | | | | | |
| | M2A8F4; | | | | | |
| | M2AXD3; | | | | | |
| | M2AYM3; | | | | | |
| | M2B2U6 | | | | | |
| Rhodopirellula europaea SH398 | M5S0P8; | | | M5S0X3; | | |
| | M5S1M6; | | | M5S737; | | |
| | M5S5U7; | | | M5SBK4 | | |
| | M5S6S9; | | | | | |
| | M5SG70 | | | | | |
| Rhodopirellula maiorica SM1 | M5RVE8 | | | | | |
| Rhodopirellula sallentina SM41 | M5TWB3; | | | M5U7D9; | | |
| | M5U479 | | | M5U8J8 | | |
| Rhodopirellula sp. SWK7 | M5T6Y4; | | | M5SZA0; | | |
| | M5TEQ7; | | | M5T4M3; | | |
| | M5TER8 | | | M5T917; | | |
| | | | | M5TC56; | | |
| | | | | M5TCY2; | | |
| | | | | M5TEW9; | | |
| | | | | M5TMK6 | | |
| Rhodopseudomonas palustris | Q21BJ6 | | | | | |
| (strain BisB18) | | | | | | |
| Rhodopseudomonas palustris | E6VPA1 | | | | | |
| (strain DX-1) | | | | | | |
| Rhodothermus marinus (strain | D0MH05; | | | D0MHK2 | | |
| ATCC 43812/DSM 4252/R-10) | D0MHK3; | | | | | |
| (Rhodothermus obamensis) | D0MHK8 | | | | | |
| Rhodothermus marinus | G2SG49 | | | G2SDQ3; | | |
| SG0.5JP17-172 | | | | G2SG43 | | |
| Rivularia sp. PCC 7116 | K9RP51 | | | | | |
| Roseburia hominis (strain DSM | | | | G2SYN7 | | |
| 16839/NCIMB 14029/A2- | | | | | | |
| 183) | | | | | | |
| Roseburia intestinalis L1-82 | C7G8W3; | | | | | |
| | C7G9B5 | | | | | |
| Roseburia intestinalis XB6B4 | D4L1G8 | | | | | |
| Roseburia sp. CAG:100 | | | | R7R0R6 | | |
| Roseburia sp. CAG:18 | | | | R5UJN2 | | |
| Roseburia sp. CAG:303 | | | | R7IML5; | | |
| | | | | R7IQE8; | | |
| | | | | R7IQI1; | | |
| | | | | R7IVZ4 | | |
| Roseburia sp. CAG:309 | | | | R6YI41; | | |
| | | | | R6YNI9 | | |
| Roseomonas cervicalis ATCC | D5RI74 | | | | | |
| 49957 | | | | | | |
| Ruminococcus albus | | Q52644 | | | | |
| Ruminococcus albus (strain | E6UAU6; | E6UBP6 | | E6UAL8; | E6UCB3 | |
| ATCC 27210/DSM 20455/ | E6UFI1; | | | E6UGE9 | | |
| JCM 14654/NCDO 2250/7) | E6UGC8; | | | | | |
| | E6UHQ2 | | | | | |
| Ruminococcus albus 8 | E9SDY0; | E9SA77 | | | | |
| | E9SF11; | | | | | |
| | E9SFJ8; | | | | | |
| | F6LP79 | | | | | |
| Ruminococcus champanellensis | D4LAD4; | | | | | |
| (strain DSM 18848/JCM 17042/ | D4LDI7 | | | | | |
| 18P13) | | | | | | |
| Ruminococcus flavefaciens | P29126 | P29126; | | | | |
| | | Q53317; | | | | |
| | | Q9S310 | | | | |
| Ruminococcus sp. CAG:177 | | | | | | R6I6M3 |
| Ruminococcus sp. CAG:382 | | | | | R6VSE8 | |
| Ruminococcus sp. CAG:488 | | | | R5YVC6 | | R5Y2E2 |
| Ruminococcus sp. CAG:563 | | | | R6DMW0 | | R6E5S2 |
| Ruminococcus sp. CAG:60 | | | | R5HWU4 | | |
| Ruminococcus sp. CAG:724 | | | | R5Q0W9 | | |
| Runella slithyformis (strain | F8EQT7; | | F8EQ23 | F8EL80; | | F8EQ73 |
| ATCC 29530/DSM 19594/ | F8EQW6; | | | F8EQP0; | | |
| LMG 11500/NCIMB 11436/ | F8EQW9 | | | F8EQX7 | | |
| LSU 4) | | | | | | |
| Saccharomonospora azurea | H8G5R6 | | | | | |
| NA-128 | | | | | | |
| Saccharomonospora azurea | H0K2F2 | | | | | |
| SZMC 14600 | | | | | | |
| Saccharomonospora cyanea | H5XHV5 | | | | | |
| NA-134 | | | | | | |
| Saccharomonospora glauca K62 | I1CXX6 | | | | | |
| Saccharomonospora | G4J3I9; | | | | | |
| paurometabolica YIM 90007 | G4J3N0 | | | | | |
| Saccharomonospora viridis | C7MUP5 | | | | | |
| (strain ATCC 15386/DSM | | | | | | |
| 43017/JCM 3036/NBRC | | | | | | |
| 12207/P101) | | | | | | |
| Saccharophagus degradans | Q21EL2; | | | Q21MN1 | | |
| (strain 2-40/ATCC 43961/ | Q21GI8; | | | | | |
| DSM 17024) | Q21HD6; | | | | | |
| | Q21NZ2; | | | | | |
| | Q21PD4 | | | | | |
| Saccharopolyspora sp. S582 | E1APH5 | | | | | |
| Salmonella typhi | | | | | Q8Z289 | |
| Salmonella typhimurium (strain | | | | | Q8ZLB7 | |
| LT2/SGSC1412/ATCC 700720) | | | | | | |
| Sanguibacter keddieii (strain | D1BHP9 | | | | | |
| ATCC 51767/DSM 10542/ | | | | | | |
| NCFB 3025/ST-74) | | | | | | |
| Scheffersomyces stipitis (strain | A3LSQ3 | | | | | |
| ATCC 58785/CBS 6054/NBRC | | | | | | |
| 10063/NRRL Y-11545) (Yeast) | | | | | | |
| (Pichia stipitis) | | | | | | |
| Scheffersomyces stipitis (strain | A3LSQ3 | | | | | |
| ATCC 58785/CBS 6054/NBRC | | | | | | |
| 10063/NRRL Y-11545) (Yeast) | | | | | | |
| (Pichia stipitis) | | | | | | |
| Scheffersomyces stipitis (Yeast) | | Q9Y7F2 | | | | |
| (Pichia stipitis) | | | | | | |
| Schizophyllum commune (Split | | P35809 | | | | |
| gill fungus) | | | | | | |
| Schizophyllum commune | D8PPF8; | D8PN69 | D8Q9M6; | D8PK12; | | |
| (strain H4-8/FGSC 9210) (Split | D8Q1J8; | | D8QFF6; | D8PKZ3; | | |
| gill fungus) | D8Q2R3; | | D8QFF9 | D8PL55; | | |
| | D8Q5U6; | | | D8PNG6; | | |
| | D8QIH9 | | | D8PQM4; | | |
| | | | | D8PT40; | | |
| | | | | D8PT41; | | |
| | | | | D8PZ92; | | |
| | | | | D8PZG2; | | |
| | | | | D8Q157; | | |
| | | | | D8Q3Z4; | | |
| | | | | D8Q3Z9; | | |
| | | | | D8Q784; | | |
| | | | | D8Q8V4; | | |
| | | | | D8Q921; | | |
| | | | | D8Q963; | | |
| | | | | D8QEP7; | | |
| | | | | D8QH17 | | |
| Sclerotinia sclerotiorum (strain | A7E5D4; | A7EQZ6; | | A7F3S6 | | |
| ATCC 18683/1980/Ss-1) | A7F2P3 | A7EXM7 | | | | |
| (White mold) (Whetzelinia | | | | | | |
| sclerotiorum) | | | | | | |
| Scytalidium thermophilum | | Q766V1 | | | | |
| Serpula lacrymans var. | F8QIE4 | | F8Q357; | F8PJ36; | | |
| lacrymans (strain S7.3) (Dry rot | | | F8QAA5 | F8PMD8 | | |
| fungus) | | | | | | |
| Serpula lacrymans var. | F8NWX6 | | F8P232; | F8NI78; | | |
| lacrymans (strain S7.9) (Dry rot | | | F8P944 | F8NKQ9 | | |
| fungus) | | | | | | |
| Setaria italica (Foxtail millet) | K3XFG1; | | | | | |
| (Panicum italicum) | K3XG02; | | | | | |
| | K3XH84; | | | | | |
| | K3ZEN0; | | | | | |
| | K3ZQ79; | | | | | |
| | K4A7W2; | | | | | |
| | K4AJ91 | | | | | |
| Setosphaeria turcica (Northern | | Q70T28; | | | | |
| leaf blight fungus) (Exserohilum | | Q9UVZ3 | | | | |
| turcicum) | | | | | | |
| Setosphaeria turcica (strain | R0I618; | R0ICV4; | R0K472; | R0I6L8; | | R0KCY5 |
| 28A) (Northern leaf blight | R0IGD8; | R0JW69; | R0KHW8 | R0ICT6; | | |
| fungus) (Exserohilum turcicum) | R0J3E9; | R0JWH8; | | R0IE39; | | |
| | R0JX99; | R0K182; | | R0JXX5; | | |
| | R0KGL9 | R0K9J6 | | R0K648; | | |
| | | | | R0K7I3; | | |
| | | | | R0K9L1; | | |
| | | | | R0KAQ2; | | |
| | | | | R0KAS6; | | |
| | | | | R0KI48; | | |
| | | | | R0KSV1 | | |
| Shewanella baltica (strain | | | | B8EEN8; | | |
| OS223) | | | | B8EEQ0; | | |
| | | | | B8EEQ1 | | |
| Shewanella putrefaciens (strain | | | | E6XKM7 | | |
| 200) | | | | | | |
| Shewanella putrefaciens (strain | | | | A4Y735; | | |
| CN-32/ATCC BAA-453) | | | | A4Y739; | | |
| | | | | A4Y751 | | |
| Shewanella sp. (strain ANA-3) | | | | A0KWY0; | | |
| | | | | A0KWY8; | | |
| | | | | A0KWY9; | | |
| | | | | A0KWZ3; | | |
| | | | | A0KWZ4 | | |
| Shewanella sp. (strain MR-4) | | | | Q0HIP5; | | |
| | | | | Q0HIP8; | | |
| | | | | Q0HIP9; | | |
| | | | | Q0HIR2 | | |
| Shewanella sp. (strain MR-7) | | | | Q0HV74; | | |
| | | | | Q0HV86; | | |
| | | | | Q0HV87; | | |
| | | | | Q0HV90 | | |
| Shewanella sp. (strain W3-18-1) | | | | A1RJD9; | | |
| | | | | A1RJF1; | | |
| | | | | A1RJF5 | | |
| Solibacter usitatus (strain | Q01YB0; | | | Q01Y63; | | Q02D65 |
| Ellin6076) | Q023N8; | | | Q022X3; | | |
| | Q024A6 | | | Q022X7 | | |
| Sorangium cellulosum | A6XB89; | | | | | |
| (Polyangium cellulosum) | A6XB90; | | | | | |
| | A6XB91; | | | | | |
| | A6XB92; | | | | | |
| | A6XB93; | | | | | |
| | A6XB94; | | | | | |
| | A6XB95; | | | | | |
| | A6XB96; | | | | | |
| | A6XB97; | | | | | |
| | A6XB98; | | | | | |
| | G3EGG2 | | | | | |
| Sorangium cellulosum (strain | A9ER43; | A9EQP3; | | | | |
| So ce56) (Polyangium | A9F9G9; | A9EUR0; | | | | |
| cellulosum (strain So ce56)) | A9F9J8; | A9G323; | | | | |
| | A9FW62; | A9GEL6 | | | | |
| | A9GMS2; | | | | | |
| | A9GSV7 | | | | | |
| Sordaria macrospora (strain | F7VY73; | F7VLK3; | | | | |
| ATCC MYA-333/DSM 997/ | F7W2U0; | F7W2U2 | | | | |
| K(L3346)/K-hell) | F7W4I1; | | | | | |
| | F7W4V6; | | | | | |
| | F7W731 | | | | | |
| Sphaerochaeta globosa (strain | | | | F0RZC1 | | |
| ATCC BAA-1886/DSM 22777/ | | | | | | |
| Buddy) (Spirochaeta sp. (strain | | | | | | |
| Buddy)) | | | | | | |
| Sphingobacterium sp. (strain | F4C1D2 | | | F4C2R5; | | |
| 21) | | | | F4C8Y4; | | |
| | | | | F4C8Y5; | | |
| | | | | F4CAC9; | | |
| | | | | F4CAU4; | | |
| | | | | F4CBD9; | | |
| | | | | F4CBP7; | | |
| | | | | F4CC01; | | |
| | | | | F4CCC7; | | |
| | | | | F4CCP2; | | |
| | | | | F4CCQ9; | | |
| | | | | F4CFP7 | | |
| Sphingobacterium sp. TN19 | D8L2X7; | | | | | |
| | D8L2Y2 | | | | | |
| Sphingobium | F6EU40 | | | F6F2Q6 | | |
| chlorophenolicum L-1 | | | | | | |
| Sphingobium indicum B90A | I5BDV0 | | | | | |
| Sphingobium japonicum (strain | D4Z2D1 | | | | | |
| NBRC 101211/UT26S) | | | | | | |
| Sphingobium japonicum BiD32 | | | | N1MIN7 | | |
| Sphingobium sp. AP49 | J2D5H9 | | | J2WIZ5 | | |
| Sphingobium yanoikuyae ATCC | K9CMD9; | | | K9CYU7 | | |
| 51230 | K9CZI3; | | | | | |
| | K9DIE1 | | | | | |
| Sphingomonas sp. LH128 | | | | J8SI09 | | |
| Sphingomonas sp. MM-1 | | | | M4RZ61 | | |
| Sphingomonas sp. S17 | F3WSK4 | | | F3X2F6 | | |
| Sphingomonas sp. SKA58 | | | | Q1NF05 | | |
| Sphingopyxis alaskensis (strain | | | | Q1GV49; | | |
| DSM 13593/LMG 18877/ | | | | Q1GV63; | | |
| RB2256) (Sphingomonas | | | | Q1GV65 | | |
| alaskensis) | | | | | | |
| Spirochaeta caldaria (strain | F8F0B3 | | | F8F4B6 | F8F1B1 | |
| ATCC 51460/DSM 7334/H1) | | | | | | |
| Spirochaeta thermophila (strain | E0RP41; | | | | | |
| ATCC 49972/DSM 6192/RI | E0RP42; | | | | | |
| 19.B1) | E0RPX5; | | | | | |
| | E0RS15; | | | | | |
| | E0RTS4 | | | | | |
| Spirochaeta thermophila (strain | G0GAM6; | | G0GAH3 | G0GB04; | | |
| ATCC 700085/DSM 6578/Z- | G0GD23; | | | G0GD76; | | |
| 1203) | G0GDR7; | | | G0GFH2 | | |
| | G0GFH0 | | | | | |
| Spirosoma linguale (strain ATCC | D2QDL1; | | | D2QDL3; | D2QMX5 | D2QHX5; |
| 33905/DSM 74/LMG 10896) | D2QE60; | | | D2QFC3; | | D2QMY2 |
| | D2QMX0; | | | D2QFH6; | | |
| | D2QU83 | | | D2QFN0; | | |
| | | | | D2QHJ5; | | |
| | | | | D2QMY6; | | |
| | | | | D2QP61; | | |
| | | | | D2QTB1; | | |
| | | | | D2QUA6 | | |
| Sporisorium reilianum (strain | E6ZPT3; | | | | | |
| SRZ2) (Maize head smut | E7A3D3 | | | | | |
| fungus) | | | | | | |
| Stackebrandtia nassauensis | | | D3Q9V8 | D3PZP9; | | |
| (strain DSM 44728/NRRL B- | | | | D3Q0Y9; | | |
| 16338/NBRC 102104/LLR- | | | | D3Q1S7; | | |
| 40K-21) | | | | D3Q2R5; | | |
| | | | | D3Q7A4 | | |
| Stanieria cyanosphaera (strain | K9XS72 | | | | | |
| ATCC 29371/PCC 7437) | | | | | | |
| Stigmatella aurantiaca (strain | E3FIR8; | | Q091X3; | E3FEB9; | | |
| DW4/3-1) | Q094N0; | | Q09DH4 | E3FIN9; | | |
| | Q09E20 | | | E3FKH8; | | |
| | | | | E3FU61; | | |
| | | | | Q08PV7; | | |
| | | | | Q08YV8 | | |
| Streptococcus anginosus | | | | | E7GY99 | |
| 1_2_62CV | | | | | | |
| Streptomyces acidiscabies | B7T8J2 | | | | | |
| Streptomyces ambofaciens | A0AD65 | | | | | |
| ATCC 23877 | | | | | | |
| Streptomyces avermitilis | Q9X584 | | | | | |
| Streptomyces avermitilis (strain | Q81ZY7; | | | | | |
| ATCC 31267/DSM 46492/ | Q82DJ2 | | | | | |
| JCM 5070/NCIMB 12804/ | | | | | | |
| NRRL 8165/MA-4680) | | | | | | |
| Streptomyces bingchenggensis | D7C253; | D7CCK0 | | D7BUE9; | | D7BVZ4 |
| (strain BCW-1) | D7C254; | | | D7C7G9 | | |
| | D7C6G6; | | | | | |
| | D7C774; | | | | | |
| | D7C775; | | | | | |
| | D7CDL1 | | | | | |
| Streptomyces bottropensis | B7T8N1 | | | | | |
| Streptomyces bottropensis | M3DE41; | M3D596 | | | | |
| ATCC 25435 | M3FRV8 | | | | | |
| Streptomyces cattleya (strain | F8JK59 | | | | | |
| ATCC 35852/DSM 46488/ | | | | | | |
| JCM 4925/NBRC 14057/NRRL | | | | | | |
| 8057) | | | | | | |
| Streptomyces chartreusis | | K4MLL9 | | P82594 | | |
| Streptomyces chattanoogensis | Q9X583 | | | | | |
| Streptomyces coelicoflavus | H1Q708; | H1Q8T6; | | H1Q8N5; | | |
| ZG0656 | H1QTR4 | H1QQ89 | | H1QDI6 | | |
| Streptomyces coelicolor (strain | Q8CJQ1; | Q9RI72; | | Q9KXY8 | | |
| ATCC BAA-471/A3(2)/M145) | Q9RJ91 | Q9RKN6 | | | | |
| Streptomyces costaricanus | | G0XSW2; | | | | |
| | | G1DTC7 | | | | |
| Streptomyces davawensis JCM | K4QSI7; | K4QXB0 | | | | |
| 4913 | K4QUN3; | | | | | |
| | K4QWE2; | | | | | |
| | K4R5P9; | | | | | |
| | K4R5R5 | | | | | |
| Streptomyces europaeiscabiei | B7T8K9 | | | | | |
| Streptomyces flavogriseus | E8W0S2; | E8W5Z2 | E8W9L1 | E8W1Y4; | | |
| (strain ATCC 33331/DSM | E8W0Y8; | | | E8W3P9; | | |
| 40990/IAF-45CD) | E8W4J1 | | | E8WBJ6 | | |
| Streptomyces fradiae | A7TVD4 | | | | | |
| (Streptomyces roseoflavus) | | | | | | |
| Streptomyces gancidicus BKS | M3BY92; | M3E0X0 | | | | |
| 13-15 | M3E8F8 | | | | | |
| Streptomyces ghanaensis ATCC | D6A1G4; | D5ZRU9; | | D6A581; | | |
| 14672 | D6A4N5; | D6A1K1 | | D6A5Q0 | | |
| | D6A6L7 | | | | | |
| Streptomyces | F3NBX5; | F3NGI4 | | | | |
| griseoaurantiacus M045 | F3NIZ6; | | | | | |
| | F3NJM9 | | | | | |
| Streptomyces griseoflavus | D9XK50; | D9XZP1 | | D9XJX5 | | |
| Tu4000 | D9Y0M5; | | | | | |
| | D9Y0M6 | | | | | |
| Streptomyces griseus XylebKG-1 | | | | G0PTB5 | | |
| Streptomyces halstedii | Q59922 | | | | | |
| Streptomyces himastatinicus | D9WKJ2; | D9WNB8 | | D9WUM9 | | |
| ATCC 53653 | D9WMU7; | | | | | |
| | D9WT61 | | | | | |
| Streptomyces hygroscopicus | H2JS44; | H2JS43 | | | | |
| subsp. jinggangensis (strain | H2K2E3 | | | | | |
| 5008) | | | | | | |
| Streptomyces hygroscopicus | M1MIJ1; | M1NED4 | | | | |
| subsp. jinggangensis TL01 | M1N8P4 | | | | | |
| Streptomyces ipomoeae 91-03 | L1KQ68; | L1KXE4 | | | | |
| | L1L6H2; | | | | | |
| | L1L7Z3 | | | | | |
| Streptomyces lasaliensis | B6ZK52 | | | | | |
| Streptomyces lividans | P26514 | P26220; | | | | |
| | | P26515 | | | | |
| Streptomyces lividans TK24 | D6EN39; | D6EHA7; | | D6EEM1 | | |
| | D6EYK6 | D6EJB3 | | | | |
| Streptomyces megasporus | D5J9N6; | | | | | |
| | F2VRZ1 | | | | | |
| Streptomyces olivaceoviridis | Q7SI98 | A4K8J7; | | | | |
| (Streptomyces corchorusii) | | Q9EW89 | | | | |
| Streptomyces pristinaespiralis | B5H6E4; | | | D6X6H6; | | |
| ATCC 25486 | B5H6V7; | | | D6X6I1 | | |
| | B5H8Y9 | | | | | |
| Streptomyces rameus | | K7UAM8 | | | | |
| Streptomyces rimosus subsp. | L8EU06 | | | | | |
| rimosus ATCC 10970 | | | | | | |
| Streptomyces scabies (strain | C9YUZ2; | C9Z2V1 | | | | |
| 87.22) (Streptomyces scabiei) | C9YVP9; | | | | | |
| | C9YW88; | | | | | |
| | C9ZB10; | | | | | |
| | C9ZE95 | | | | | |
| Streptomyces scabies | B7T8I4 | | | | | |
| (Streptomyces scabiei) | | | | | | |
| Streptomyces sp. C | D9VMD8; | | | D9W3R6 | | |
| | D9VMH4 | | | | | |
| Streptomyces sp. e14 | D6KFT7 | D6K459 | | | | |
| Streptomyces sp. EC3 | | Q56013 | | | | |
| Streptomyces sp. NH | I7CZR6 | | | | | |
| Streptomyces sp. PAMC26508 | M9TIB3; | M9TK95 | | M9U718 | | |
| | M9TLF5; | | | | | |
| | M9U3X1 | | | | | |
| Streptomyces sp. S27 | C3RYK8 | D1FNQ6 | | | | |
| Streptomyces sp. S38 | | Q59962 | | | | |
| Streptomyces sp. S9 | B4XVN1 | D7EZJ3 | | | | |
| Streptomyces sp. SirexAA-E | G2NAD2 | G2NBA0 | | G2NGY1; | | |
| | | | | G2NK26; | | |
| | | | | G2NK77; | | |
| | | | | G2NMK2 | | |
| Streptomyces sp. SPB78 | | | | | | D9UNB5 |
| Streptomyces sp. SWU10 | F2Z9L1 | F7J663; | | | | |
| | | F8WSY7 | | | | |
| Streptomyces sp. THW31 | | E5L391 | | | | |
| Streptomyces sp. TN119 | C6FX34 | K9JD34 | | | | |
| Streptomyces sp. Tu6071 | | | F3ZHF2 | | | F3Z693; |
| | | | | | | F3ZH49 |
| Streptomyces sp. zxy19 | | B0ZSE5 | | | | |
| Streptomyces stelliscabiei | B7T8I9 | | | | | |
| Streptomyces sviceus ATCC | B5HPL8; | | | B5HW70 | | |
| 29083 | B5HRG8; | | | | | |
| | B5HZ14; | | | | | |
| | B5I0S5; | | | | | |
| | B5I430 | | | | | |
| Streptomyces tendae | Q7X2C9 | | | | | |
| Streptomyces | C6ZHB0 | | | | | |
| thermocarboxydus | | | | | | |
| Streptomyces | Q9RMM5 | Q9RMM4 | | | | |
| thermocyaneoviolaceus | | | | | | |
| Streptomyces thermoviolaceus | Q76BV3 | Q76BV2 | | | | |
| Streptomyces thermovulgaris | | B2KJ43 | | | | |
| Streptomyces turgidiscabies | Q5IK56 | | | | | |
| Streptomyces turgidiscabies | L7EV41; | L7EST2 | | | | |
| Car8 | L7F2F6; | | | | | |
| | L7F547; | | | | | |
| | L7F7B2; | | | | | |
| | L7FCB0; | | | | | |
| | L7FDD1 | | | | | |
| Streptomyces venezuelae | F2RHS3; | | | | | |
| (strain ATCC 10712/CBS | F2RHT2; | | | | | |
| 650.69/DSM 40230/JCM | F2RHT8 | | | | | |
| 4526/NBRC 13096/PD 04745) | | | | | | |
| Streptomyces violaceusniger Tu | G2NZ34 | | G2PBJ0 | G2NU37; | G2P1R8 | |
| 4113 | | | | G2NU38; | | |
| | | | | G2NUW7; | | |
| | | | | G2P3B1; | | |
| | | | | G2P4X5; | | |
| | | | | G2PFQ3 | | |
| Streptomyces | D9XAI6; | | | D9X8M3 | | |
| viridochromogenes DSM 40736 | D9XDG3; | | | | | |
| | D9XGV4 | | | | | |
| Streptomyces | L8P5Y2; | | | L8P510; | | |
| viridochromogenes Tue57 | L8P6E6; | | | L8P9Y6; | | |
| | L8PHD7; | | | L8PCD0 | | |
| | L8PP58 | | | | | |
| Streptomyces viridosporus | | Q9RMH9 | | | | |
| Streptosporangium roseum | D2B806 | | | | | |
| (strain ATCC 12428/DSM | | | | | | |
| 43021/JCM 3005/NI 9100) | | | | | | |
| Synechococcus elongatus | Q31ND9 | | | | | |
| (strain PCC 7942) (Anacystis | | | | | | |
| nidulans R2) | | | | | | |
| Synechococcus sp. (strain ATCC | Q5N5S3 | | | | | |
| 27144/PCC 6301/SAUG | | | | | | |
| 1402/1) (Anacystis nidulans) | | | | | | |
| Synechococcus sp. PCC 7335 | B4WP63 | | | | | |
| Synechocystis sp. PCC 6803 | | L8ALW6 | | L8ARK8 | | |
| Talaromyces pinophilus | | G9DBG3 | | | | |
| Talaromyces stipitatus (strain | B8M9H8; | B8MEX2; | | B8MTM2 | | |
| ATCC 10500/CBS 375.48/QM | B8MH80 | B8MND2; | | | | |
| 6759/NRRL 1006) (Penicillium | | B8MTU7 | | | | |
| stipitatum) | | | | | | |
| Talaromyces thermophilus | | M4VJR2 | | | | |
| Tannerella sp. CAG:118 | | | R5IG66 | R5I8B1 | | |
| Tepidanaerobacter | | | | | F4LUH4 | |
| acetatoxydans (strain DSM | | | | | | |
| 21804/JCM 16047/Re1) | | | | | | |
| Teredinibacter turnerae (strain | C5BKG0; | C5BMU2; | | C5BI48; | C5BJ89 | C5BK66; |
| ATCC 39867/T7901) | C5BLA7; | C5BQU7; | | C5BK78; | | C6AR15 |
| | C5BN19; | C5BU24 | | C5BKF9; | | |
| | C5BPD1; | | | C5BKG2; | | |
| | C5BPK1; | | | C5BSM4; | | |
| | C5BPL7; | | | C5BT64 | | |
| | C5BQL3; | | | | | |
| | C5BQQ4; | | | | | |
| | C5BRL9; | | | | | |
| | C5BTG8 | | | | | |
| Terriglobus roseus (strain DSM | I3ZEB2; | | | | | |
| 18391/NRRL B-41598/KBS | I3ZFY1 | | | | | |
| 63) | | | | | | |
| Terriglobus saanensis (strain | E8V5N9; | | | | | E8V227 |
| ATCC BAA-1853/DSM 23119/ | E8V6J8 | | | | | |
| SP1PR4) | | | | | | |
| Thalassiosira oceanica (Marine | K0SY78 | | | | | |
| diatom) | | | | | | |
| Thalassiosira pseudonana | B8C511 | | | | | |
| (Marine diatom) (Cyclotella | | | | | | |
| nana) | | | | | | |
| Thanatephorus cucumeris | L8WW62; | | L8WNN3 | L8WL57 | | |
| (strain AG1-IA) (Rice sheath | L8WX40; | | | | | |
| blight fungus) (Rhizoctonia | L8WYA7 | | | | | |
| solani) | | | | | | |
| Thanatephorus cucumeris | M5BQL3; | | | M5C1V5; | | |
| (strain AG1-IB/isolate 7/3/14) | M5BSB2; | | | M5CA29; | | |
| (Lettuce bottom rot fungus) | M5C4S7; | | | M5CGI7 | | |
| (Rhizoctonia solani) | M5C787; | | | | | |
| | M5CB49; | | | | | |
| | M5CBU8; | | | | | |
| | M5CDA1; | | | | | |
| | M5CE19; | | | | | |
| | M5CH99 | | | | | |
| Thermoanaerobacter italicus | D3T5Y5; | | | | | |
| (strain DSM 9252/Ab9) | D3T5Y9 | | | | | |
| Thermoanaerobacter mathranii | D7ARC1; | | | | | |
| (strain DSM 11426/CIP 108742/ | D7ARC5 | | | | | |
| A3) | | | | | | |
| Thermoanaerobacter | D2X5N2; | | | | | P36906 |
| saccharolyticum | E5KBL2; | | | | | |
| | P36917 | | | | | |
| Thermoanaerobacter | M8CYB5 | | | | | |
| thermohydrosulfuricus WC1 | | | | | | |
| Thermoanaerobacter | Q60046 | | | | | |
| thermosulfurogenes | | | | | | |
| (Clostridium | | | | | | |
| thermosulfurogenes) | | | | | | |
| Thermoanaerobacterium | Q60043 | | | | | |
| Thermoanaerobacterium | I3VVC1; | I3VTR8 | | I3VRU5 | | I3VVB4 |
| saccharolyticum (strain DSM | I3VVC2 | | | | | |
| 8691/JW/SL-YS485) | | | | | | |
| Thermoanaerobacterium sp. | | | | | | O30360 |
| (strain JW/SL YS485) | | | | | | |
| Thermoanaerobacterium | D9TMZ9; | | | D9TT82 | | D9TT77 |
| thermosaccharolyticum (strain | D9TN00 | | | | | |
| ATCC 7956/DSM 571/NCIB | | | | | | |
| 9385/NCA 3814) (Clostridium | | | | | | |
| thermosaccharolyticum) | | | | | | |
| Thermoanaerobacterium | L0IK21 | | | | | |
| thermosaccharolyticum M0795 | | | | | | |
| Thermoanaerobacterium | F6BIF7; | | | | | |
| xylanolyticum (strain ATCC | F6BIF8 | | | | | |
| 49914/DSM 7097/LX-11) | | | | | | |
| Thermoascus aurantiacus | P23360 | | | | | |
| Thermobacillus composti | L0EAT5; | L0E9J8; | | L0EC29 | | |
| (strain DSM 18247/JCM 13945/ | L0EF86; | L0EBB6 | | | | |
| KWC4) | L0EGW1; | | | | | |
| | L0EGW5 | | | | | |
| Thermobacillus xylanilyticus | O69261 | Q14RS0 | | | | |
| Thermobaculum terrenum | D1CC70 | | | D1CH80; | | D1CI48 |
| (strain ATCC BAA-798/YNP1) | | | | D1CHR8 | | |
| Thermobifida alba | P74912 | | | | | |
| Thermobifida fusca (strain YX) | Q47KR6; | Q47QL8 | | | | |
| | Q47L48 | | | | | |
| Thermobifida halotolerans | | I3NRT9 | | | | |
| Thermobispora bispora (strain | D6Y2K1; | D6Y4B1 | | | | |
| ATCC 19993/DSM 43833/CBS | D6Y5E0 | | | | | |
| 139.67/JCM 10125/NBRC | | | | | | |
| 14880/R51) | | | | | | |
| Thermomonospora curvata | | | | D1A4I8; | | |
| (strain ATCC 19995/DSM | | | | D1A6V4 | | |
| 43183/JCM 3096/NCIMB | | | | | | |
| 10081) | | | | | | |
| Thermomonospora fusca | | Q56265; | | | | |
| | | Q5RZ98 | | | | |
| Thermomyces lanuginosus | | F8UV78; | | | | |
| (Humicola lanuginosa) | | O43097 | | | | |
| Thermophilic anaerobe NA10 | O24820 | | | | | |
| Thermopolyspora flexuosa | Q8GMV6 | Q8GMV7 | | | | |
| Thermosynechococcus | Q8DHP3 | | | | | |
| elongatus (strain BP-1) | | | | | | |
| Thermotoga lettingae (strain | A8F6C7 | | | | | |
| ATCC BAA-301/DSM 14385/ | | | | | | |
| TMO) | | | | | | |
| Thermotoga maritima | Q7WUM6; | | | | | |
| | Q7WVV0 | | | | | |
| Thermotoga maritima (strain | G4FGX6; | | | | | |
| ATCC 43589/MSB8/DSM | Q60037; | | | | | |
| 3109/JCM 10099) | Q9WXS5 | | | | | |
| Thermotoga naphthophila | D2C750; | | | | | |
| (strain ATCC BAA-489/DSM | D2C759 | | | | | |
| 13996/JCM 10882/RKU-10) | | | | | | |
| Thermotoga neapolitana | Q60041; | | | | | |
| | Q60042; | | | | | |
| | Q79C18 | | | | | |
| Thermotoga neapolitana (strain | B9K766; | | | | | |
| ATCC 49049/DSM 4359/NS- | B9K775; | | | | | |
| E) | B9K945 | | | | | |
| Thermotoga petrophila (strain | A5IL00; | | | A5IKD4; | | |
| RKU-1/ATCC BAA-488/DSM | A5IL09 | | | A5IKD6 | | |
| 13995) | | | | | | |
| Thermotoga sp. | Q60044 | | | | | |
| Thermotoga sp. (strain RQ2) | B1LA81; | | | B1L9L7; | | |
| | B1LA89; | | | Q7WU65 | | |
| | B1LC77 | | | | | |
| Thermotoga sp. EMP | J9H0U8; | | | | | |
| | J9HCV0 | | | | | |
| Thermotoga sp. strain FjSS3-B.1 | Q9R6T4; | | | | | |
| | Q9WWJ9 | | | | | |
| Thermotoga thermarum DSM | F7YVM4; | | | F7YX80 | | |
| 5069 | F7YXD6 | | | | | |
| Thielavia heterothallica (strain | G2Q7T8; | G2Q4M3; | G2Q1N4; | G2Q562; | | |
| ATCC 42464/BCRC 31852/ | G2QG07; | G2Q4S6; | G2QA11; | G2Q7W6; | | |
| DSM 1799) (Myceliophthora | G2QGN6; | G2Q913; | G2QEB0 | G2QAJ6; | | |
| thermophila) | G2QJ91 | G2QDB9; | | G2QCC8; | | |
| | | G2QIK8; | | G2QDD9; | | |
| | | G2QIR3; | | G2QDZ0; | | |
| | | G2QIR4; | | G2QFK0; | | |
| | | G2QNI1 | | G2QFK1; | | |
| | | | | G2QGR9; | | |
| | | | | G2QHQ6; | | |
| | | | | G2QHQ9; | | |
| | | | | G2QM97; | | |
| | | | | G2QQ09 | | |
| Thielavia terrestris (strain ATCC | G2QSH7; | G2QUC8; | G2R8F8; | G2QRB5; | | G2QYV6; |
| 38088/NRRL8126) | G2QVE8; | G2QV82; | G2RHB5; | G2QRB8; | | G2QYV7 |
| (Acremonium alabamense) | G2QXD2; | G2QWT6; | G2RHE1 | G2R1A0; | | |
| | G2R5G6; | G2QYN6; | | G2R283; | | |
| | G2R8G4; | G2R747 | | G2R299; | | |
| | G2R8T7 | | | G2R6X6; | | |
| | | | | G2R7Z2; | | |
| | | | | G2RD72; | | |
| | | | | G2RDN5 | | |
| Togninia minima (strain UCR- | R8BCE5 | | R8BIG3; | R8BK88 | | R8BQW6 |
| PA7) (Esca disease fungus) | | | R8BTX6 | | | |
| (Phaeoacremonium | | | | | | |
| aleophilum) | | | | | | |
| Treponema azotonutricium | F5YDP7; | | | | | |
| (strain ATCC BAA-888/DSM | F5YDP8 | | | | | |
| 13862/ZAS-9) | | | | | | |
| Treponema saccharophilum | | | | H7EPH5 | | |
| DSM 2985 | | | | | | |
| Treponema sp. JC4 | I0XCR4 | | | | | |
| Treponema succinifaciens | F2NWU1 | | | | | |
| (strain ATCC 33096/DSM 2489/ | | | | | | |
| 6091) | | | | | | |
| Trichoderma asperellum | | Q6QNU8 | | | | |
| Trichoderma harzianum | | B5A7N4; | Q8J0I9 | | | |
| (Hypocrea lixii) | | P48793 | | | | |
| Trichoderma longibrachiatum | | F8W669 | | | | |
| Trichoderma pseudokoningii | B0FXL9 | B0FXM0 | | | | |
| Trichoderma sp. SC9 | | D2XV89 | | | | |
| Trichoderma sp. SY | | Q8J0T4 | | | | |
| Truepera radiovictrix (strain | D7CRC3; | | | D7CRC2; | | |
| DSM 17093/CIP 108686/LMG | D7CRC9 | | | D7CTK1 | | |
| 22925/RQ-24) | | | | | | |
| Tsukamurella paurometabola | D5UQ92 | | | | | |
| (strain ATCC 8368/DSM 20162/ | | | | | | |
| JCM 10117/NBRC 16120/ | | | | | | |
| NCTC 13040) (Corynebacterium | | | | | | |
| paurometabolum) | | | | | | |
| Uncinocarpus reesii (strain | | | | C4JQ75 | | |
| UAMH 1704) | | | | | | |
| Ustilago hordei (strain Uh4875- | I2FVS0; | I2FN07 | | | | |
| 4) (Barley covered smut fungus) | I2FWP8 | | | | | |
| Ustilago maydis (strain 521/ | Q4P641; | Q4P0L3 | | | | |
| FGSC 9021) (Corn smut fungus) | Q4P902 | | | | | |
| Verrucomicrobiae bacterium | B5JGI9; | | | | | |
| DG1235 | B5JHG2; | | | | | |
| | B5JHQ9; | | | | | |
| | B5JLG2; | | | | | |
| | B5JLG3; | | | | | |
| | B5JLL0; | | | | | |
| | B5JLR7 | | | | | |
| Verrucosispora maris (strain | F4F343; | F4FB94 | | F4F6N4; | | |
| AB-18-032) | F4F3H8; | | | F4FD00 | | |
| | F4F899; | | | | | |
| | F4FAW9; | | | | | |
| | F4FBX5; | | | | | |
| | F4FE45 | | | | | |
| Verticillium albo-atrum (strain | C9SCH5; | C9SCF4; | | C9SET9 | | |
| VaMs.102/ATCC MYA-4576/ | C9SMV7; | C9SNM9; | | | | |
| FGSC 10136) (Verticillium wilt) | C9SXL0 | C9SNN0 | | | | |
| Verticillium dahliae (strain | G2WZE3; | G2X0L1; | | G2X0C9 | | |
| VdLs.17/ATCC MYA-4575/ | G2X0N0; | G2X4G0; | | | | |
| FGSC 10137) | G2X407; | G2X4G1; | | | | |
| | G2XDP1 | G2X5X8 | | | | |
| Verticillium dahliae | | Q0ZHI9 | | | | |
| (Verticillium wilt) | | | | | | |
| Volvariella volvacea | Q7Z948 | | | | | |
| Volvox carteri (Green alga) | D8U3T4 | | | | | |
| Xanthomonas axonopodis pv. | Q8PET6; | | | | P58935 | |
| citri (strain 306) | Q8PEU1 | | | | | |
| Xanthomonas axonopodis pv. | G2M0D9; | | | | | |
| citrumelo F1 | G2M0E4 | | | | | |
| Xanthomonas axonopodis pv. | K8FY50; | | | | | |
| malvacearum str. GSPB1386 | K8G2F2 | | | | | |
| Xanthomonas axonopodis pv. | K8FRZ7 | | | | | |
| malvacearum str. GSPB2388 | | | | | | |
| Xanthomonas axonopodis pv. | | | H1XIU2 | | | |
| punicae str. LMG 859 | | | | | | |
| Xanthomonas axonopodis | M4U3F2; | | | | | |
| Xac29-1 | M4U3F7 | | | | | |
| Xanthomonas campestris pv. | Q4UNX5; | | | | | |
| campestris (strain 8004) | Q4UNX8 | | | | | |
| Xanthomonas campestris pv. | Q8P3F3 | | | | | |
| campestris (strain ATCC 33913/ | | | | | | |
| NCPPB 528/LMG 568) | | | | | | |
| Xanthomonas campestris pv. | B0RZ11; | | | | | |
| campestris (strain B100) | B0RZ14 | | | | | |
| Xanthomonas campestris pv. | G0CA22; | | | | | |
| raphani 756C | G0CA25 | | | | | |
| Xanthomonas campestris pv. | Q3BMC2; | | | | | |
| vesicatoria (strain 85-10) | Q3BMC7 | | | | | |
| Xanthomonas citri pv. | H8FE49; | | H8FLQ7 | | | |
| mangiferaeindicae LMG 941 | H8FE52; | | | | | |
| | H8FE54 | | | | | |
| Xanthomonas citri subsp. citri | M4VV28; | | | | | |
| Aw12879 | M4VV34 | | | | | |
| Xanthomonas fuscans subsp. | D4TAU1; | | | | | |
| aurantifolii str. ICPB 10535 | D4TAU6 | | | | | |
| Xanthomonas fuscans subsp. | D4SV44; | | | | | |
| aurantifolii str. ICPB 11122 | D4SV49 | | | | | |
| Xanthomonas gardneri ATCC | F0C0I1; | | | F0C0B0 | | |
| 19865 | F0C2V4 | | | | | |
| Xanthomonas perforans 91-118 | F0BRT8; | | | | | |
| | F0BRU2 | | | | | |
| Xanthomonas translucens | L7GMT7 | | | | | |
| DAR61454 | | | | | | |
| Xanthomonas translucens pv. | K8Z0Y9 | | | | | |
| graminis ART-Xtg29 | | | | | | |
| Xanthomonas translucens pv. | L0SV97 | | | | | |
| translucens DSM 18974 | | | | | | |
| Xanthomonas vesicatoria ATCC | F0BDW7 | | | | | |
| 35937 | | | | | | |
| Xylanimicrobium pachnodae | Q9RQB7 | Q9RQB8 | | | | |
| Xylanimonas cellulosilytica | D1BRX2; | D1BXH1 | | D1BXA0 | | D1BTZ1 |
| (strain DSM 15894/CECT 5975/ | D1BWB1; | | | | | |
| LMG 20990/XIL07) | D1BXQ6; | | | | | |
| | D1BXQ7 | | | | | |
| Yersinia pseudotuberculosis | | | | | B2K6N0 | |
| serotype IB (strain PB1/+) | | | | | | |
| Zobellia galactanivorans (strain | G0L7J0; | | | | | |
| DSM 12802/CIP 106680/ | G0L7J1; | | | | | |
| NCIMB 13871/Dsij) | G0L8X3 | | | | | |
| Zunongwangia profunda (strain | D5BGE4; | | D5BC68 | D5BAV6 | | |
| DSM 18752/CCTCC AB 206139/ | D5BGE5; | | | | | |
| SM-A87) | D5BHG0 | | | | | |
| Zymomonas mobilis subsp. | | | | | I6YGE6 | |
| mobilis ATCC 29191 | | | | | | |

### REFERENCES

1. Aachary A A, Prapulla S G. 2011. Xylooligosaccharides (XOS) as an emerging prebiotic: microbial synthesis, utilization, structural characterization, bioactive properties, and applications. Compreh. Rev. Food Sci. 10:2-16.
2. Vazquez M J, Alonso J L, Dominguez H, Parajo J C. 2000. Xylooligosaccharides: manufacture and applications. Trends Food Sci. Technol. 11:387-393.
3. Otieno D O, Ahring B K. 2012. The potential for oligosaccharide production from the hemicellulose fraction of biomasses through pretreatment processes: xylooligosaccharides (XOS), arabinooligosaccharides (AOS), and mannooligosaccharides (MOS). Carbohydr. Res. 360:84-92.
4. Chen H H, Chen Y K, Chang H C, Lin S Y. 2012. Immunomodulatory effects of xylooligosaccharides. Food Sci. Technol. Res. 18:195-199.
5. Ebringerova A, Hromadkova Z. 1999. Xylans of industrial and biomedical importance. Biotechnol. Genet. Eng. Rev. 16:325-346.
6. Anderson V R, Perry C M. 2006. Pentosan polysulfate: a review of its use in the relief of bladder pain or discomfort in interstitial cystitis. Drugs 66:821-835.
7. Schuchman E H, Ge Y, Lai A, Borisov Y, Faillace M, Eliyahu E, He X X, Iatridis J, Vlassara H, Striker G, Simonaro C M. 2013. Pentosan polysulfate: a novel therapy for the mucopolysaccharidoses. PLoS One. 8: e54459. doi:10.1371/journal.pone.0054459.
8. Goodrich L R, Nixon A J. 2006. Medical treatment of osteoarthritis in the horse—A review. Vet. J. 171:51-69.
9. Carpita N C. 1996. Structure and biogenesis of the cell walls of grasses. Annu Rev. Plant Physiol. Plant Mol. Biol. 47:445-476.
10. Ebringerova A, Hromadkova Z, Heinze T. 2005. Hemicellulose. Adv. Polym. Sci. 186:1-67.
11. Biely P, Vrsanska M, Tenkanen M, Kluepfel D. 1997. Endo-beta-1,4-xylanase families: differences in catalytic properties. J. Biotechnol. 57:151-166.
12. Preston J, Hurlbert J, Rice J, Ragunathan A, St John F. 2003. Microbial strategies for the depolymerization of glucuronoxylan: Leads to biotechnological applications of endoxylanases. Application of enzymes to lignocellulosics 855:191-210.
13. Collins T, Gerday C, Feller G. 2005. Xylanases, xylanase families and extremophilic xylanases. FEMS Microbiol. Rev. 29:3-23.
14. St John F, Rice J, Preston J. 2006. Paenibacillus sp strain JDR-2 and XYNA1: a novel system for methylglucuronoxylan utilization. Appl. Environ. Microbiol. 72:1496-1506.
15. St John F, Hurlbert J, Rice J, Preston J, Pozharski E. 2011. Ligand bound structures of a glycosyl hydrolase family 30 glucuronoxylan xylanohydrolase. J. Mol. Biol. 407:92-109.
16. St John F J, Rice J D, Preston J F. 2006. Characterization of XYNC from Bacillus subtilis subsp. subtilis strain 168 and analysis of its role in depolymerization of glucuronoxylan. J. Bacteriol. 188:8617-8626.
17. St John F J, Gonzalez J M, Pozharski E. 2010. Consolidation of glycosyl hydrolase family 30: A dual domain 4/7 hydrolase family consisting of two structurally distinct groups. FEBS Lett. 584:4435-4441.
18. Tjalsma H, Antelmann H, Jongbloed J D H, Braun P G, Darmon E, Dorenbos R, Dubois J Y F, Westers H, Zanen G, Quax W J, Kuipers O P, Bron S, Hecker M, van Dijl J M. 2004. Proteomics of protein secretion by Bacillus subtilis: separating the “secrets” of the secretome. Microbiol. Mol. Biol. Rev. 68:207-233.
19. Ay J, Gotz F, Borriss R, Heinemann U. 1998. Structure and function of the Bacillus hybrid enzyme GluXyn-1: Native-like jellyroll fold preserved after insertion of autonomous globular domain. Proc. Natl. Acad. Sci. U.S.A. 95:6613-6618.
20. Vandermarliere E, Bourgois T M, Rombouts S, Van Campenhout S, Volckaert G, Strelkov S V, Delcour J A, Rabijns A, Courtin C M. 2008. Crystallographic analysis shows substrate binding at the −3 to +1 active-site subsites and at the surface of glycoside hydrolase family 11 endo-1,4-beta-xylanases. Biochem. J. 410:71-79.
21. Schallmey M, Singh A, Ward O P. 2004. Developments in the use of Bacillus species for industrial production. Can. J. Microbiol. 50:1-17.
22. Pohl S, Harwood C R. 2010. Heterologous protein secretion by Bacillus Species: from the cradle to the grave. Adv. Appl. Microbiol. 73:1-25.
23. Spizizen J. 1958. Transformation of biochemically deficient strains of Bacillus subtilis by deoxyribonucleate. Proc. Natl. Acad. Sci. U.S.A. 44:1072-1078.
24. Rhee M S, Kim J W, Qian Y L, Ingram L O, Shanmugam K T. 2007. Development of plasmid vector and electroporation condition for gene transfer in sporogenic lactic acid bacterium, Bacillus coagulans. Plasmid 58:13-22.
25. Hurlbert J, Preston J. 2001. Functional characterization of a novel xylanase from a corn strain of Erwinia chrysanthemi. J. Bacteriol. 183:2093-2100.
26. Dubois M, Gilles K A, Hamilton J K, Rebers P A, Smith F. 1956. Colormetric method for determination of sugars and related substances. Anal. Chem. 28:350-356.
27. Blumenkrantz. N, Asboehan. G. 1973. New method for quantitative determination of uronic acids. Anal. Biochem. 54:484-489.
28. Nelson N. 1944. A photometric adaptation of the Somogyi method for the determination of glucose. J. Biol. Chem. 153:375-380.
29. Kenealy W R, Jeffries T W. 2003. Rapid 2,2′-bicinchoninic-based xylanase assay compatible with high throughput screening. Biotechnol. Lett. 25:1619-1623.
30. Zhou S D, Ingram L O. 2001. Simultaneous saccharification and fermentation of amorphous cellulose to ethanol by recombinant Klebsiella oxytoca SZ21 without supplemental cellulase. Biotechnol. Lett. 23:1455-1462.
31. Kardosova A, Matulova M, Malovikova A. 1998. (4-O-Methyl-alpha-D-glucurono)-D-xylan from Rudbeckia fulgida, var. sullivantii (Boynton et Beadle). Carbohydr. Res. 308:99-105.
32. Zuobi-Hasona K, St. John F, Rice J D, Preston J F. 2001. Oligosaccharides containing glucuronoxylose as substrates for selecting bacteria for depolymerization of hemicellulose. p. 534. Abstr. 101^{st} Natl. Meet. Am. Soc. Microbiol., Orlando, Fla. American Society for Microbiology, Washington, D.C.
33. Cavagna F, Deger H, Puls J. 1984. 2D-NMR analysis of the structure of an aldotriouronic acid obtained from birch wood. Carbohydr. Res. 129:1-8.
34. Koutaniemi S, Guillon F, Tranquet O, Bouchet B, Tuomainen P, Virkki L, Petersen H L, Willats W G T, Saulnier L, Tenkanen M. 2012. Substituent-specific antibody against glucuronoxylan reveals close association of glucuronic acid and acetyl substituents and distinct labeling patterns in tree species. Planta 236:739-751.
35. Chow V, Nong G, Preston J. 2007. Structure, function, and regulation of the aldouronate utilization gene cluster from Paenibacillus sp strain JDR-2. J. Bacteriol. 189:8863-8870.
36. Nishitani K, Nevins D J. 1991. Glucuronoxylan xylanohydrolase—a unique xylanase with requirement for appendant glucuronosyl units. J. Biol. Chem. 266:6539-6543.
37. Rice J, Nong G, Ragunathan A, St. John F, Preston J F. 2005. Depolymerization processes catalyzed by the GH5 endoxylanase secreted by Erwinia chrysanthemi PI. p.52. Abstr. 105^{th} Natl. Meet. Am. Soc. Microbiol., Atlanta. Ga. American Society for Microbiology, Washington, D.C.
38. Vrsanska M, Kolenova K, Puchart V, Biely P. 2007. Mode of action of glycoside hydrolase family 5 glucuronoxylan xylanohydrolase from Erwinia chrysanthemi. FEBS J. 274:1666-1677.
39. Ebringerova A, Kardosova A, Hromadkova Z, Malovikova A, Hribalova V. 2002. Immunomodulatory activity of acidic xylans in relation to their structural and molecular properties. Int. J. Biol. Macromol. 30:1-6.
40. Christakopoulos P, Katapodis P, Kalogeris E, Kekos D, Macris BJ, Stamatis H, Skaltsa H. 2003. Antimicrobial activity of acidic xylo-oligosaccharides produced by family 10 and 11 endoxylanases. Int. J. Biol. Macromol. 31:171-175.
41. Kwan C, Bell R, Koenig T, Bischofberger A, Horadagoda N, Perkins N R, Jeffcott L B, Dart A J. 2012. Effects of intra-articular sodium pentosan polysulfate and glucosamine on the cytology, total protein concentration and viscosity of synovial fluid in horses. Aust. Vet. J. 90:315-320.
42. Daus S, Petzold-Welcke K, Kötteritzsch M, Baumgaertel A, Schubert U S, Heinze T. 2011. Homogeneous Sulfation of Xylan from Different Sources. Macromolecular Materials and Engineering 296:551-561.
43. Bryan E M, Bae T, Kleerebezem H, Dunny G M. 2000. Improved vectors for nisin-controlled expression in gram-positive bacteria. Plasmid 44:183-190.
44. Wilson A C, Perego M, Hoch J A. 2007. New transposon delivery plasmids for insertional mutagenesis in Bacillus anthracis. J. Microbiol. Methods 71:332-335.

1-33. (canceled)

34 A genetically modified microorganism comprising genetic modifications to:
a) a gene encoding a secreted endoxylanase belonging to glycoside hydrolase family 10 or a homolog thereof, if present;
and genetic modifications to one or more of:
b) a gene encoding a secreted endoxylanasc belonging to glycoside hydrolase family 11 or a homolog thereof, and/or
c) a gene encoding a secreted endoxylanase belonging to glycoside hydrolase family 30 or a homolog thereof, and
optionally, express a gene encoding a secreted alpha-glucuronidase glycoside hydrolase family 67 or a homolog thereof, and/or a gene encoding a secreted endoxylanase belonging to glycoside hydrolase family 115 or a homolog thereof,
and wherein, said genetic modifications inactivate the enzymatic activity of the endoxylanases produced by said target gene.

35. The genetically modified microorganism of claim 34, comprising genetic modifications to:
a) the gene encoding the secreted endoxylanase belonging to glycoside hydrolase family 10 or the homolog thereof and the gene encoding the secreted endoxylanase belonging to glycoside hydrolase family 11 or the homolog thereof and, optionally, the gene encoding the secreted endoxylanase belonging to glycoside hydrolase family 30 or the homolog thereof;
b) the gene encoding the secreted endoxylanase belonging to glycoside hydrolase family 10 or the homolog thereof and the gene encoding the secreted endoxylanase belonging to glycoside hydrolase family 30 or the homolog thereof and, optionally, the gene encoding the secreted endoxylanase belonging to glycoside hydrolase family 11 or the homolog thereof;
c) the gene encoding the secreted endoxylanase belonging to glycoside hydrolase family 11 or the homolog thereof and the gene encoding the secreted endoxylanase belonging to glycoside hydrolase family 30 or the homolog thereof, provided that said microorganism lacks a functional secreted endoxylanase belonging to glycoside hydrolase family 10 or a homolog thereof;
d) the gene encoding the secreted endoxylanase belonging to glycoside hydrolase family 30 or the homolog thereof, provided that said microorganism lacks a functional secreted endoxylanase belonging to glycoside hydrolase family 10 or a homolog thereof; or
e) the gene encoding the secreted endoxylanase belonging to glycoside hydrolase family 11 or the homolog thereof, provided that said microorganism lacks a functional secreted endoxylanase belonging to glycoside hydrolase family 10 or a homolog thereof, wherein, culturing the genetically modified organism in the presence of methylglucuronoxylans (MeGXₙ) produces aldopentauronate methylglucuronoxylotetraose (MeGX₄), aldotetrauronate 4-O-methylglucuronoxylotriose (MeGX₃), and/or aldotetrauronate 4-O-methylglucuronoxybiose (MeGX₂).

36. The genetically modified microorganism of claim 34, comprising genetic modifications to:
a) the gene encoding a secreted endoxylanase belonging to glycoside hydrolase family 10 or a homolog thereof, the gene encoding a secreted endoxylanase belonging to glycoside hydrolase family 11 or a homolog thereof and the gene encoding a secreted endoxylanase belonging to glycoside hydrolase family 30 or a homolog thereof; or
b) the gene encoding a secreted endoxylanase belonging to glycoside hydrolase family 11 or a homolog thereof and the gene encoding a secreted endoxylanase belonging to glycoside hydrolase family 30 or a homolog thereof, provided that said microorganism lacks a functional secreted endoxylanase belonging to glycoside hydrolase family 10 or a homolog thereof,
and wherein, culturing the genetically modified organism in the presence of methylglucuronoxylans (MeGXₙ) produces aldopentauronate methylglucuronoxylotetraose (MeGX₄), xylotriose (X₃), xylobiose (X₂), and/or xylose (X₁).

37. The genetically modified microorganism of claim 34, further comprising genetic modifications to one or more genes encoding proteins belonging to glycoside hydrolase family 43 (GH43), glycoside hydrolase family 8 (GH8), and/or glycoside hydrolase family 39 (GH39).

38. The genetically modified microorganism of claim 34, wherein the organism is Bacillus subtilis.

39. The genetically modified microorganism of claim 34, wherein the organism is Paenibacillus sp. JDR2.

40. A genetically modified B. subtilis strain 168 comprising genetic modifications to:
a) a gene encoding a secreted endoxylanase belonging to glycoside hydrolase family 11 or a homolog thereof, and/or
b) a gene encoding a secreted endoxylanase belonging to glycoside hydrolase family 30 or a homolog thereof,
and wherein, said genetic modifications inactivate the enzymatic activity of the secreted endoxylanases produced by said target genes.

41. The genetically modified B. subtilis strain 168 of claim 40, comprising genetic modifications to the gene encoding the secreted endoxylanase belonging to glycoside hydrolase family 11 or the homolog thereof,
wherein, culturing the genetically modified B. subtilis strain 168 in the presence of methylglucuronoxylans (MeGXₙ) produces aldopentauronate methylglucuronoxylotetraose (MeGX₄), aldotetrauronate 4-O-methylglucuronoxylotriose (MeGX₃), and/or aldotetrauronate 4-O-methylglucuronoxybiose (MeGX₂).

42. The genetically modified B. subtilis strain 168 of claim 40, comprising genetic modifications to the gene encoding a secreted endoxylanase belonging to glycoside hydrolase family 30 or a homolog thereof,
and wherein, culturing the genetically modified B. subtilis strain 168 in the presence of methylglucuronoxylans (MeGXₙ) produces aldopentauronate methylglucuronoxylotetraose (MeGX₄), xylotriose (X₃), xylobiose (X₂), and/or xylose (X₁).

43. The B. subtilis strain 168 of claim 40, further comprising genetic modifications to one or more genes encoding proteins belonging to glycoside hydrolase family 43 (GH43), glycoside hydrolase family 8 (GH8), and/or glycoside hydrolase family 39 (GH39).

44. A method of producing xylooligosaccharides without arabinofuranosyl substitutions (XOS), xylooligosaccharides with arabinofuranosyl substitutions (AXOS), acidic xylooligosaccharides without arabinofuranosyl substitutions (U-XOS), and/or acidic xylooligosaccharides with arabinofuranosyl substitutions (U-AXOS), the method comprising:
a) culturing the genetically modified microorganism of claim 34 in a culture medium comprising methylglucuronoxylans (MeGXₙ) and/or methylglucronoarabinoxylans (MeGAXn) under conditions that allow conversion of MeGXₙ and/or MeGAXₙ to XOS, AXOS, U-XOS, and/or U-AXOS, and
b) optionally, purifying XOS, AXOS, U-XOS, and/or U-AXOS from the culture medium.

45. The method of claim 44, wherein the genetically modified microorganism comprises genetic modifications to:
a) the gene encoding a secreted endoxylanase belonging to glycoside hydrolase family 10 or a homolog thereof; and
b) the gene encoding a secreted endoxylanase belonging to glycoside hydrolase family 11 or a homolog thereof,
and wherein, culturing the genetically modified organism in presence methylglucuronoxylans (MeGXₙ) produces aldopentauronate methylglucuronoxylotetraose (MeGX₄), aldotetrauronate 4-O-methylglucuronoxylotriose (MeGX₃), and/or aldotetrauronate 4-O-methylglucuronoxybiose (MeGX₂).

46. The method of claim 44, wherein the genetically modified microorganism comprises genetic modifications to:
a) the gene encoding a secreted endoxylanase belonging to glycoside hydrolase family 10 or a homolog thereof; and
b) the gene encoding a secreted endoxylanase belonging to glycoside hydrolase family 30 or a homolog thereof,
and wherein, culturing the genetically modified organism in the presence of methylglucuronoxylans (MeGXₙ) produces aldopentauronate methylglucuronoxylotetraose (MeGX₄), xylotriose (X₃), xylobiose (X₂), and/or xylose (X₁).

47. The method of claim 44, wherein the genetically modified microorganism is B. subtilis strain 168 comprising genetic modifications to the gene encoding a secreted endoxylanase belonging to glycoside hydrolase family 11 or a homolog thereof,
and wherein, culturing the genetically modified B. subtilis strain 168 in the presence of methylglucuronoxylans (MeGXₙ) produces aldopentauronate methylglucuronoxylotetraose (MeGX₄), aldotetrauronate 4-O-methylglucuronoxylotriose (MeGX₃), and/or aldotetrauronate 4-O-methylglucuronoxybiose (MeGX₂).

48. The method of claim 44, wherein the genetically modified microorganism is B. subtilis strain 168 comprising genetic modifications to the gene encoding a secreted endoxylanase belonging to glycoside hydrolase family 30 or a homolog thereof,
and wherein, culturing the genetically modified organism in the presence of methylglucuronoxylans (MeGXₙ) produces aldopentauronate methylglucuronoxylotetraose (MeGX₄), xylotriose (X₃), xylobiose (X₂), and/or xylose (X₁).

49. A nutraceutical or pharmaceutical composition comprising XOS, AXOS, U-XOS, and/or U-AXOS produced by the method of claim 44.

50. The neutraceutical or pharmaceutical composition of claim 49, wherein U-XOS or U-AXOS are sulfated.

51. The neutraceutical or pharmaceutical composition of claim 49, wherein U-XOS is aldouronates, U-XOS containing one or more methylglucuronate residues linked α-1,2 to xylose residues in the β-1,4-xylan backbone in methylglucuronoxylans, and/or pentosan polyslfate.

52. A method of producing xylooligosaccharides without arabinofuranosyl substitutions (XOS), xylooligosaccharides with arabinofuranosyl substitutions (AXOS), acidic xylooligosaccharides without arabinofuranosyl substitutions (U-XOS), and/or acidic xylooligosaccharides with arabinofuranosyl substitutions (U-AXOS), the method comprising:
a) culturing the genetically modified microorganism of claim 34 in a culture medium comprising methylglucuronoxylans (MeGXₙ) and/or methylglucronoarabinoxylans (MeGAXₙ) under conditions that allow conversion of MeGXₙ and/or MeGAXₙ to XOS, AXOS, U-XOS, and/or U-AXOS, and
b) optionally, purifying XOS, AXOS, U-XOS, and/or U-AXOS from the culture medium.

53. The method of claim 52, wherein the genetically modified microorganism comprises genetic modifications to:
a) the gene encoding a secreted endoxylanase belonging to glycoside hydrolase family 10 or a homolog thereof; and
b) the gene encoding a secreted endoxylanase belonging to glycoside hydrolase family 11 or a homolog thereof,
and wherein, culturing the genetically modified organism in presence methylglucuronoxylans (MeGXₙ) produces aldopentauronate methylglucuronoxylotetraose (MeGX₄), aldotetrauronate 4-O-methylglucuronoxylotriose (McGX₃), and/or aldotetrauronate 4-O-methylglucuronoxybiose (MeGX₂).

54. The method of claim 52, wherein the genetically modified microorganism comprises genetic modifications to:
a) the gene encoding a secreted endoxylanase belonging to glycoside hydrolase family 10 or a homolog thereof; and
b) the gene encoding a secreted endoxylanase belonging to glycoside hydrolase family 30 or a homolog thereof,
and wherein, culturing the genetically modified organism in the presence of methylglucuronoxylans (MeGXₙ) produces aldopentauronate methylglucuronoxylotetraose (MeGX₄), xylotriose (X₃), xylobiose (X₂), and/or xylose (X₁).

55. The method of claim 52, wherein the genetically modified microorganism is B. subtilis strain 168 comprising genetic modifications to the gene encoding a secreted endoxylanase belonging to glycoside hydrolase family 11 or a homolog thereof, and wherein, culturing the genetically modified B. subtilis strain 168 in the presence of methylglucuronoxylans (MeGXₙ) produces aldopentauronate methylglucuronoxylotetraose (MeGX₄), aldotetrauronate 4-O-methylglucurono xylotriose (MeGX₃), and/or aldotetrauronate 4-O-methylglucuronoxybiose (MeGX₂).


SEQUENCE LISTING (10 sequences)

SEQ ID NO: 1 - DNA, 32 bp, Artificial Sequence
  forward primer used for the amplification of xynD-xynC-bglC′ genes from B. subtilis strain 168
gcatacctcgagcgtctggcaatggcggtgta

SEQ ID NO: 2 - DNA, 21 bp, Artificial Sequence
  reverse primer used for the amplification of xynD-xynC-bglC′ genes from B. subtilis strain 168
agcagcagcaatctacaacct

SEQ ID NO: 3 - DNA, 32 bp, Artificial Sequence
  forward primer used in the amplification of DNA containing xynA genes from B. subtilis strain 168
ggagtgctcgagaggaggaagtcatggtaagc

SEQ ID NO: 4 - DNA, 35 bp, Artificial Sequence
  forward primer used in the amplification of DNA containing xynA genes from B. subtilis strain 168
gcgttgtctagatcgtagagtccccattcataaat

SEQ ID NO: 5 - DNA, 34 bp, Artificial Sequence
  forward primer used for the amplification of DNA containing GH11 endoxylanase xynA gene from B. subtilis strain 168
atgtccctcgagagcacagactactggcaaaatt

SEQ ID NO: 6 - DNA, 32 bp, Artificial Sequence
  reverse primer used for the amplification of DNA containing GH11 endoxylanase  xynA gene from B. subtilis strain 168
cgataaggatcccctacctccagcaattccaa

SEQ ID NO: 7 - protein, 311 aa, Bacillus subtilis
MQQDPSISSKQKSTAVIKMVISMVIFGSIGFFSEHTNLPSFELVFVRCLCATLFLGFCWLASGQYKTEKWSKRDVLQTLACGFFLVFNWVFLFKSFEETSVTIAISVYHLAPVLVLLLGSFFYREKLNVISVSSIIICFLGTALISGINGSTSLTQLMGSGIIWAVLAALFYAFTTLLGKGIHNLSPYTTTFLQTGLGVIILIPFIHFGAFADLSQGNWIMVVSTGIIHTGIVYLLFFDSLRFLSTKFISIIVFLDPAVAIVLDTVFTGFRPDLYQTLGIVMIFAGMALTLVRRQGKANVTAEGTDIEQIQ

SEQ ID NO: 8 - DNA, 936 bp, Bacillus subtilis
atgcaacaggatccttccatatcgtcaaaacaaaaatcaacagccgtaatcaaaatggtcatctctatggtgattttcggctccatcggctttttctcagagcataccaatctgccgtcatttgaattggtctttgtccgctgcctttgcgcgacgctgtttttaggcttttgctggctggcgtccggccagtataaaactgaaaaatggagcaagagagacgtgctgcaaacattggcgtgcggtttctttcttgtattcaactgggtgttcttatttaagtcttttgaagaaacgtccgttacaattgccatctctgtttatcatctagcgccggtgcttgttcttcttctggggagcttcttctacagagagaaattaaatgtgatctccgtcagctccattatcatctgctttcttgggactgcgctgatttccgggattaacggcagcacatcattgacacagctgatgggatcagggatcatatgggcagttctcgccgcactgttttatgcgttcaccactttattgggaaaaggcattcacaatctcagtccttacacgaccaccttcttgcagacaggtttaggggttatcatcctgattccgtttattcactttggcgcttttgcggacctatctcagggaaactggattatggtggtatctaccggcatcatccatacgggtattgtatatttgctgttttttgacagtttacgatttttatcaacgaaatttatttcaatcattgtatttctcgaccccgctgtcgcgatcgtgctcgataccgtcttcacaggcttccgccctgacctctatcaaacgcttggcatcgtaatgatctttgcgggcatggccttgacgcttgtcaggaggcaggggaaggcgaatgtgacagctgagggtacggatattgaacaaatacaataa

SEQ ID NO: 9 - protein, 428 aa, Bacillus subtilis
MRKYQARIISIILAMIFIMFWDYLFYFIGKNPINWPVDIVYTAVTLVSVWMLAYYIDEKQQLVKKMKDNEWKYKQLSEEKNRIMDNLQEIVFQTNAKGEITYLNQAWASITGFSISECMGTMYNDYFIKEKHVADHINTQIQNKASSGMFTAKYVTKNGTIFWGEVHYKLYYDRDDQFTGSLGTMSDITERKEAEDELIEINERLARESQKLSITSELAAGIAHEVRNPLTSVSGFLQIMKTQYPDRKDYFDIIFSEIKRIDLVLSELLLLAKPQAITFKTHQLNEILKQVTTLLDTNAILSNIVIEKNFKETDGCMINGDENQLKQVFINIIKNGIEAMPKGGVVTISTAKTASHAVISVKDEGNGMPQEKLKQIGKPFYSTKEKGTGLGLPICLRILKEHDGELKIESEAGKGSVFQVVLPLKSDS

SEQ ID NO: 10 - DNA, 1287 bp, Bacillus subtilis
atgagaaaatatcaagctcgtatcatttccatcattttggcaatgatttttataatgttttgggattatttattttattttataggcaaaaacccgattaattggcctgttgatattgtgtatactgcagtcacgctggtaagtgtctggatgttggcttattatattgatgagaaacagcagctggttaagaaaatgaaggataacgaatggaagtataaacagctttctgaagaaaaaaaccgcatcatggataatttgcaggaaatcgtatttcaaacgaatgcaaaaggtgaaattacatatttaaaccaagcgtgggcatctataaccgggttttcaatcagtgaatgtatgggaacaatgtataacgattacttcataaaagaaaagcacgtagccgaccacattaacacccaaatccaaaacaaagcgtcttctggcatgtttacggcaaaatacgtgacaaaaaacggcacgattttttggggagaagttcattataaactttactatgaccgggatgaccaatttacaggcagcctgggtacaatgtcagatatcactgagcggaaagaggctgaagatgagctcattgagattaatgaacggctggcgagggaatcccagaaactatcaatcacaagtgaacttgccgcaggtattgctcatgaggtcagaaaccctttaacatctgtcagcggtttcctccagattatgaaaacacaatatccggacagaaaagactattttgacatcatcttttcagagattaaaagaatcgatttagtgctcagcgagctgctgctgcttgcaaaaccgcaggcaatcacatttaaaacacaccagcttaatgagatcttgaaacaagtcacgacattgcttgataccaatgcaattctgtccaatatcgtcatagagaaaaatttcaaagagacagatggctgtatgattaatggagacgaaaatcagctgaagcaggtctttatcaacatcattaaaaacggaattgaggcaatgccaaagggcggtgtcgtaaccatttcaactgctaaaaccgcctctcatgcagtgataagcgtaaaggatgaaggaaacggcatgccgcaggaaaagctgaagcagattggcaaacctttttattcaacaaaagaaaagggcactggactgggacttcccatttgtttgagaatcctgaaggaacatgacggggaattgaaaatcgaaagtgaagctggaaaaggcagcgtctttcaagtggttttgcctttaaaatcagacagctga